(19) United States
(12) Patent Application Publication
(10) Pub. No.: US 2025/0318749 A1
(43) Pub. Date: Oct. 16, 2025

(54) DEVICES, SYSTEMS, AND METHODS FOR MAKING AND USING A PARTIALLY FLUID-FILLABLE CIRCUIT

(71) Applicant: Liquid Wire Inc., Portland, OR (US)

(72) Inventors: Jorge E. Carbo, JR., Portland, OR (US); Mark William Ronay, Portland, OR (US); Michael Jasper Wallans, Portland, OR (US); Sai Srinivas Desabathina, Beaverton, OR (US)

(73) Assignee: Liquid Wire Inc., Portland, OR (US)

(21) Appl. No.: 18/999,153

(22) Filed: Dec. 23, 2024

Related U.S. Application Data
(63) Continuation of application No. PCT/US22/78823, filed on Oct. 27, 2022.
(60) Provisional application No. 63/366,778, filed on Jun. 22, 2022.

(51) Int. Cl. A61B 5/11; A61B 5/00; G01L 1/04; G01L 1/14; G01L 1/20; G01L 9/00

(52) U.S. Cl. CPC A61B 5/11; A61B 5/6807; G01L 1/04; G01L 1/146; G01L 1/205; G01L 9/0026; A61B 2560/0468; A61B 2562/0247; A61B 2562/0261; A61B 2562/168

(57) ABSTRACT

A partially fluid-fillable circuit assembly is disclosed herein. The partially fluid-fillable circuit can include a layup composed of a substrate layer; a deformable conductor; and an encapsulation layer covering the deformable conductor. A first portion of the layup can include a sealed perimeter that, along with at least one surface defined by the layup, can define a first fluid-fillable cavity. A second portion of the layup can be unitized and structurally distinguished from the cavity defined by the first portion of the layup.

### CROSS-REFERENCE TO RELATED APPLICATIONS

The present application is a continuation of PCT Application No. PCT/US22/78823, filed 27 Oct. 2022, which claims the benefit of priority from U.S. Provisional Patent Application No. 63/366,778, titled DEVICES, SYSTEMS, AND METHODS FOR MAKING AND USING A PARTIALLY FLUID FILLABLE CIRCUIT, filed Jun. 22, 2022, the disclosure of both are hereby incorporated by reference in their entirety.

### FIELD

The present disclosure is generally related to sustainable, flexible circuits and, more particularly, is directed to flexible circuits that can be either integrated with or attached onto a fluid-fillable bladder such that the flexible circuit can be operable when the bladder is inflated and/or optionally deflated.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, a partially fluid-fillable circuit assembly is disclosed. The partially fluid-fillable circuit can include a layup composed of a substrate layer; a deformable conductor; and an encapsulation layer covering the deformable conductor. A first portion of the layup can include a sealed perimeter that, along with at least one surface defined by the layup, can define a first fluid-fillable cavity. A second portion of the layup can be unitized and structurally distinguished from the cavity defined by the first portion of the layup.

These and other features and characteristics of the present disclosure, as well as the methods of operation and functions of the related elements of structure and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIG. 1 illustrates a perspective view of a fluid-fillable circuit, in accordance with one non-limiting aspect of the present disclosure;

FIG. 2 illustrates a cross-sectioned view of the fluid-fillable circuit of FIG. 1, in accordance with at least one non-limiting aspect of the present disclosure;

FIGS. 3A, 3B, 3C, and 3D collectively illustrate an assembly of the fluid-fillable circuit of FIG. 1, in accordance with at least one non-limiting aspect of the present disclosure;

FIG. 4 illustrates a perspective view of another inflatable circuit, in accordance with at least one non-limiting aspect of the present disclosure;

FIGS. 5A, 5B, 5C, and 5D collectively illustrate an assembly of the fluid-fillable circuit of FIG. 4, in accordance with at least one non-limiting aspect of the present disclosure;

FIG. 6 illustrates a perspective view of another inflatable circuit, in accordance with at least one non-limiting aspect of the present disclosure;

FIGS. 7A, 7B, 7C, and 7D collectively illustrate an assembly of the fluid-fillable circuit of FIG. 6, in accordance with at least one non-limiting aspect of the present disclosure;

FIG. 8 illustrates a perspective view of another inflatable circuit, in accordance with at least one non-limiting aspect of the present disclosure;

FIGS. 9A, 9B, 9C, and 9D collectively illustrate an assembly of the fluid-fillable circuit of FIG. 8, in accordance with at least one non-limiting aspect of the present disclosure;

FIG. 10 illustrates an assembly of another inflatable circuit featuring an auxiliary device, in accordance with at least one non-limiting aspect of the present disclosure;

FIG. 11, illustrates a perspective view of the assembled inflatable circuit of FIG. 10, in accordance with at least one non-limiting aspect of the present disclosure;

FIGS. 12A and FIG. 12B illustrate perspective views of embodiments of fluid-fillable circuits;

FIG. 13 is a graph illustrating a correlation between an electrical parameter and a structural parameter of a fluid-fillable circuit;

FIG. 14 illustrates a flow chart illustrating a method of manufacturing a fluid-fillable circuit, in accordance with at least one non-limiting aspect of the present disclosure;

FIG. 15 illustrates a flow chart illustrating a method of using a fluid-fillable circuit, in accordance with at least one non-limiting aspect of the present disclosure;

FIG. 16 illustrates a flow chart illustrating another method of using a fluid-fillable circuit, in accordance with at least one non-limiting aspect of the present disclosure;

FIG. 17 illustrates a partially fluid-fillable circuit, in accordance with at least one non-limiting aspect of the present disclosure;

FIG. 18 illustrates an assembly of the partially fluid-fillable circuit of FIG. 17, in accordance with at least one non-limiting aspect of the present disclosure;

FIG. 19 illustrates another partially fluid-fillable circuit, in accordance with at least one non-limiting aspect of the present disclosure;

FIG. 20 illustrates an assembly of the partially fluid-fillable circuit of FIG. 19, in accordance with at least one non-limiting aspect of the present disclosure;

FIG. 21 illustrates another assembly of another partially fluid-fillable circuit, in accordance with at least one non-limiting aspect of the present disclosure.

FIG. 22 illustrates another partially fluid-fillable circuit, in accordance with at least one non-limiting aspect of the present disclosure;

FIG. 23 illustrates a cross-sectioned side view of the partially fluid-fillable circuit of FIG. 22, in accordance with at least one non-limiting aspect of the present disclosure;

FIG. 24 illustrates an assembly of the partially fluid-fillable circuit of FIG. 22, in accordance with at least one non-limiting aspect of the present disclosure;

FIG. 25 illustrates another partially fluid-fillable circuit, in accordance with at least one non-limiting aspect of the present disclosure;

FIGS. 26A, 26B, and 26C illustrate an assembly of the partially fluid-fillable circuit of FIG. 25, in accordance with at least one non-limiting aspect of the present disclosure;

FIG. 27 illustrates another partially fluid-fillable circuit, in accordance with at least one non-limiting aspect of the present disclosure;

FIGS. 28A and 28B illustrate top views of a first and second layup of the partially fluid-fillable circuit of FIG. 27, in accordance with at least one non-limiting aspect of the present disclosure;

FIGS. 29A and 29B illustrate cross-sectioned views of fluid-fillable portions of the partially fluid-fillable circuit of FIG. 27, in accordance with at least one non-limiting aspect of the present disclosure;

FIGS. 30A and 30B illustrate one implementation of another partially fluid-fillable circuit, in accordance with at least one non-limiting aspect of the present disclosure;

FIG. 31 illustrates an assembly of the partially fluid-fillable circuit of FIGS. 30A and 30B, in accordance with at least one non-limiting aspect of the present disclosure;

FIG. 32 illustrates another implementation of another partially fluid-fillable circuit, in accordance with at least one non-limiting aspect of the present disclosure;

FIG. 33 illustrates a perspective view of a fluid-fillable portion of the partially fluid-fillable circuit of FIG. 32, in accordance with at least one non-limiting aspect of the present disclosure.

FIG. 34, illustrates an alternate cavity configuration configured for use with the fluid-fillable portion of FIG. 33, in accordance with at least one non-limiting aspect of the present disclosure;

FIG. 35 illustrates a plan view of a conduit configuration for use with a partially-fluid-fillable circuit, in accordance with at least one non-limiting aspect of the present disclosure;

FIG. 36 illustrates another fluid-fillable portion configured for use with the partially fluid-fillable circuit of FIG. 32, in accordance with at least one non-limiting aspect of the present disclosure;

FIGS. 37A and 37B, a such capacitive touch interface configured for use with a partially-fluid-fillable circuit is depicted in accordance with at least one non-limiting aspect of the present disclosure; and

FIG. 38 illustrates a flow chart of a method of manufacturing a substrate from which a fluid-fillable circuit can be produced, in accordance with at least one non-limiting aspect of the present disclosure.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward”, “rearward”, “left”, “right”, “upwardly”, “downwardly”, and the like are words of convenience and are not to be construed as limiting terms. Furthermore, it is to be understood that such terms as “forward”, “rearward”, “left”, “right”, “upwardly”, “downwardly”, and the like are words of convenience and are not to be construed as limiting terms.

In the following description, like reference characters designate like or corresponding parts throughout the several views of the drawings. Also in the following description, it is to be understood that such terms as “forward”, “rearward”, “left”, “right”, “upwardly”, “downwardly”, and the like are words of convenience and are not to be construed as limiting terms.

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves any and all copyrights disclosed herein.

The devices, systems, and methods disclosed in U.S. Provisional Patent Application No. 63/154,665, titled HIGHLY SUSTAINABLE CIRCUITS AND METHODS FOR MAKING THEM, filed Feb. 26, 2021, the disclosure of which is hereby incorporated by reference in its entirety, U.S. Provisional Patent Application No. 63/243,206, titled SUSTAINABLE INFLATABLE CIRCUITS AND METHODS FOR MAKING THEM, filed Sep. 13, 2021, the disclosure of which is hereby incorporated by reference in its entirety, and U.S. Provisional Patent Application No. 63/272,487, titled DEVICES, SYSTEMS, AND METHODS FOR MAKING AND USING A FLUID-FILLABLE CIRCUIT, filed Oct. 27, 2021, the disclosure of which is hereby incorporated by reference in its entirety, are relevant to the subject matter herein.

Electronic circuits that are flexible and deformable have emerged as a means of innovating conventional electronics and introducing electronics into new products and applications. However, it would be beneficial for flexible electronic circuits to form a sealed, internal cavity, which can be filled with a fluid. While certain electronic components and/or traces may have a certain degree of inherent flexibility, that flexibility is typically constrained both in the amount the components can flex, their resilience in flexing, and the number of times the electronic components can flex before the electronic components deteriorate or break. Moreover, electronics that have the ability to stretch, such as those comprising silver or other conductive inks, have insufficient durability and typically do not recover fully when subjected to elongation, resulting in ever-changing electrical characteristics until they fail completely. Consequently, the utility of such electronic components may be limited and unsuitable for constructing a fluid-fillable circuit, as they will not possess the reliability or longevity or by the ability to function when filled.

Therefore, it would be beneficial to use a conductive gel of sorts for traces in a fluid-fillable circuit, as a conductive gel can provide for electronic components that are flexible, extensible and deformable while maintaining resiliency. Some such devices, systems, and methods are disclosed in U.S. Provisional Patent Application No. 63/272,487, titled DEVICES, SYSTEMS, AND METHODS FOR MAKING AND USING A FLUID-FILLABLE CIRCUIT, filed Oct. 27, 2021, the disclosure of which is hereby incorporated by reference in its entirety. The operational flexing, stretching, deforming, or other physical manipulation of a conductive trace formed from conductive gel may produce predictable, measurable changes in the electrical characteristics of the trace with little to no hysteresis upon returning to a relaxed state. By measuring the change in resistance or impedance of such a trace the change in length of the trace may be inferred. By combining the changes in lengths of multiple traces, the relative movement of points on a two-dimensional surface may be calculated.

The relative movement of points in a three-dimensional space may be calculated and determined using two-dimensional displacement information if the points are disposed on a body that has constrained motion, for example, points located on limbs of a body that are interconnected by a joint. However, there remains a need for partially fluid-fillable circuits that feature non-fillable portions that can be integrated into a housing of the circuit and/or configured to accommodate any combination of electronic components or deformable conductors, which can be used to supplement functions provided via the fluid-fillable portions of the circuit. Accordingly, there remains a need for devices, systems, and methods for making and using a partially fluid-fillable circuit that utilize a deformable conductor.

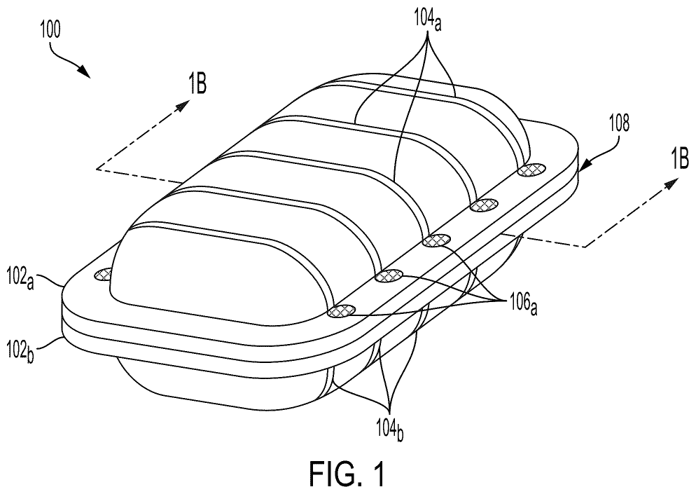

Referring now to FIG. 1, a perspective view of a fluid-fillable circuit 100 is depicted in accordance with at least one non-limiting aspect of the present disclosure. According to the non-limiting aspect of FIG. 1, the fluid-fillable circuit 100 can include a first layup 102ₐ, and a second layup 102_{b}. The first layup 102ₐ can include a first plurality of traces 104ₐ and the second layup 102_{b} can include a second plurality of traces 104. The first layup 102ₐ of FIG. 1 can be placed on top of—and mechanically coupled to—the second layup 102_{b} such that selected features-for example, the first plurality of traces 104ₐ and the second plurality of traces 104_{b}, are aligned. For example, each trace 104ₐ of the first plurality of traces 104ₐ can be electrically coupled to a corresponding trace 104_{b} of the second plurality of traces 104_{b} via a plurality of electrical vias 106ₐ and 106_{b} (not shown) positioned at a mechanical interface of the first layup 102ₐ and the second layup 102_{b}. However, in other non-limiting aspects, the first layup 102ₐ can be mechanically coupled, fused, or otherwise integrated to the second layup 102_{b} and a single plurality of traces can be deposited and/or coupled to both the first layup 102ₐ and the second layup 102_{b}, such that each trace of the plurality traverses the entire perimeter collectively defined by the first layup 102ₐ and the second layup 102_{b}.

According to the non-limiting aspect of FIG. 1, the vias 106 of the first layup 102ₐ can be dimensioned and positioned such that they substantially correspond to and align with the vias (not shown) of the second layup 102_{b}. However, in other non-limiting aspects, vias 106 of the first layup 102ₐ can be alternately configured relative to the vias 106 of the second layup 102_{b}. For example, the vias 106 of the first layup 102ₐ can be larger than the vias 106 of the second layup 102_{b} to account for tolerances and/or misalignment. Alternatively and/or additionally, the vias 106 of the first layup 102ₐ may be intentionally misaligned relative to the vias 106 of the second layup 102_{b}

Still referring to FIG. 1, the first layup 102ₐ and the second layup 102_{b} of FIG. 1 can be configured such that either the first layup 102ₐ or the second layup 102_{b} can be positioned on top of the other. However, in other non-limiting aspects, either the first layup 102ₐ or the second layup 102ₐ can be specifically configured as a top portion or a bottom portion of the fluid-fillable circuit 100. For example, it may be preferable for a top portion or a bottom portion of the fluid-fillable circuit 100 to have specifically configured dimensions, mechanical features, and/or electrical features. As such, either the first layup 102ₐ or the second layup 102_{b} can be configured to include such features, rendering them exclusively suitable for placement on the top portion or a bottom portion of the fluid-fillable circuit 100. Some non-limiting examples of “other features” that can be added to the first layup 102ₐ or the second layup 102_{b} include electronic components, portions of the trace pattern, trace breaks with vias at trace endpoints, and/or a predetermined dimension of the first layup 102ₐ or the second layup 102_{b} when the fluid-fillable circuit 100 is, e.g., either inflated or deflated, amongst others. Alternatively and/or additionally, the first layup 102ₐ of FIG. 1 can be arranged relative to the second layup 102_{b} such that other features of the first layup 102ₐ are preferably aligned with other features of the second layup 102_{b}.

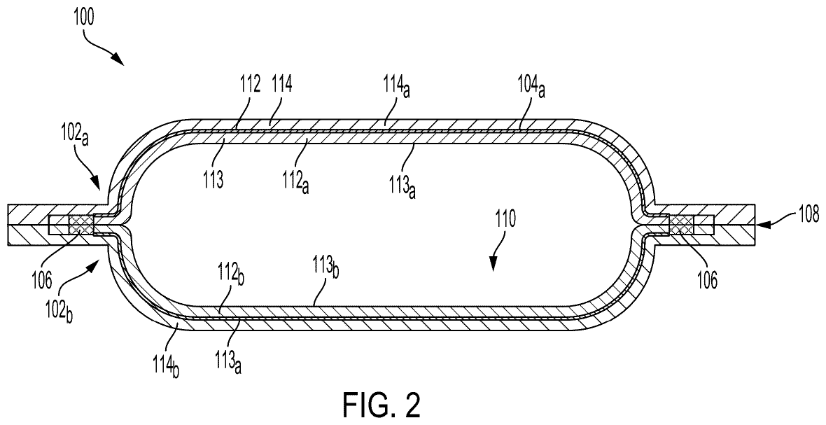

As used herein, the term “inflate” shall include the introduction of any foreign substance into an internal cavity 110 (FIG. 2) defined by the first and second layups 102ₐ, 102_{b}. For example, as used herein, “inflation” can include the introduction of a compressible fluid, a non-compressible fluid, a foam, and/or particles, amongst other media, into an internal cavity 110 (FIG. 2) of the circuit 100. Likewise, the term “deflate” shall include the removal of any foreign substance from the internal cavity 110 (FIG. 2) defined by the first and second layups 102ₐ, 102_{b}. However, according to some non-limiting aspects, a “deflated” condition of the circuit can include an initial state after forming the bladder and/or a state after fluid has been released from the bladder.

According to the non-limiting aspect of FIG, 1, the first layup 102ₐ and the second layup 102_{b} can be composed of flexible and stretchable materials, such as those disclosed by U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. Specifically, the first layup 102ₐ and the second layup 102_{b} can be fabricated from a flexible or stretchable material such as a natural rubber, a synthetic rubber, a flexible plastic, a silicone based material (e.g., polydimethylsiloxane (“PDMS”), thermoplastic polyurethane (“TPU”), ethylene propylene diene terpolymer (“EPDM”), neoprene, polyethylene terephthalate (“PET”), etc.), a flexible composite material, and/or a naturally flexible materials, such as a leather, for example. For example, the first layup 102ₐ and/or the second layup 102_{b} can be fabricated from a resilient, albeit stretchable TPU, such as Lubrizol® Estane® 58000 series (e.g., 58238), amongst others. Alternatively, the first layup 102ₐ and/or the second layup 102_{b} can be formed from a flexible, though comparatively more rigid material, such as Lubrizol® Estane® S375D, amongst others.

Since each trace 104ₐ of the first plurality of traces 104ₐ can be electrically coupled to a corresponding trace 104_{b} of the second plurality of traces 104_{b} via the plurality of electrical vias 106ₐ and 106_{b}, according to some non-limiting aspects, it may be preferable to fill the vias 106ₐ, 106_{b} with a deformable conductor configured to convey electrical energy between corresponding traces 104ₐ, 104_{b}. Such a deformable conductor is represented via the cross-hatching in FIGS. 1-3D. The deformable conductor placed in the vias 106ₐ, 106_{b} can be the same as, or similar to, the deformable conductors used for the traces 104ₐ, 104_{b}. Alternately, the deformable conductor placed in the vias 106ₐ, 106_{b} can be different than the deformable conductors used for the traces 104ₐ, 104_{b}. However, the use of cross-hatching is merely illustrative and the particular nature and/or absence of cross-hatching in any of the figures shall not be construed as limiting to the deformable conductor and/or the vias 106ₐ, 106_{b}, themselves. According to other non-limiting aspects, it shall be appreciated that the vias 106ₐ, 106_{b} can be alternately configured to convey electrical energy between corresponding traces 104ₐ, 104_{b} of the circuit 100.

As used herein, the term “deformable conductor” shall include but shall not be limited to, any conductor that is in a fluid state, or any conductor that can transition into a fluid state when an external stimulus (e.g. a strain, etc.) is applied to either the conductor or a structure surrounding the conductor, wherein the conductor is also capable of returning to a pre-stimulus state. Alternatively and/or additionally, a “deformable conductor” can include an elastic property greater than an elastic property of its surrounding structure, such that the conductor will not plastically deform before the surrounding structure plastically deforms. In other words, a “deformable conductor,” as used herein, shall include a rigidity that does not preclude the fluid-fillable circuits disclosed herein from undergoing the requisite transition between a first (e.g., deflated) state to a second (e.g., inflated) state while preserving electrical communication throughout the circuit in either the first or second state. In this regard, a “deformable conductor” is distinguished from conventional conductors, such as copper wires, which would prevent the transition between the first and second states, or whose rigidity might break the electrical communication throughout the circuit during said transitions.

In some aspects, the properties of the deformable conductive material and/or the properties of the layers surrounding the patterns of the deformable conductive material may be adjusted and/or optimized to ensure that the patterns of deformable conductive material heal upon unitization of the surrounding layers. For example, the deformable conductive material may be optimized to have a viscosity such that the deformable conductive material is able to heal upon unitization of the layers but not such that the deformable conductive material overly deforms and does not achieve the intended pattern. As another example, an adhesive characteristics and/or viscosity of the deformable conductive material may be optimized such that it remains on the substrate layer upon removal of the removable stencil 50 and but does not adhere to the channels 504, 506 of the stencil thereby lifting the deformable conductive material off of the substrate layer. In some aspects, a viscosity of the deformable conductive material may, when under high shear (e.g., in motion), be in a range of about 10 Pascal seconds (Pa*s) and 500 Pa*s, such as a range of 50 Pa*s and 300 Pa*s, and/or may be about 50 Pa*s, about 60 Pa*s, about 70 Pa*s, about 80 Pa*s, about 90 Pa*s, about 100 Pa*s, about 110 Pa*s, about 120 Pa*s, about 130 Pa*s, about 140 Pa*s, about 150 Pa*s, about 160 Pa*s, about 170 Pa*s, about 180 Pa*s, about 190 Pa*s, or about 200 Pa*s. In some aspects, a viscosity of the deformable conductive material may, when under low shear (e.g., at rest), be in a range of 1,000,000 Pa*s and 40,000,000 Pa*s and/or may be about 10,000,000 Pa*s, about 20,000,000 Pa*s, about 30,000,000 Pa*s, or about 40,000,000 Pa*s.

Similarly, the traces 104ₐ, 104_{b} of the circuit 100 of FIG. 1 can include a deformable, conductive material, such as those disclosed in International Patent Application No. PCT/US2017/019762 titled LIQUID WIRE, which was filed on Feb. 27, 2017 and published on Sep. 8, 2017 as International Patent Publication No. WO2017/151523A1, the disclosure of which is hereby incorporated by reference in its entirety. For example, each trace 104ₐ, 104_{b} can include a variety of forms, such as a liquid, a paste, a gel, and/or a powder, amongst others that would enable the traces 104ₐ, 104_{b} to have a deformable (e.g., soft, flexible, stretchable, bendable, clastic, flowable viscoelastic, Newtonian, non-Newtonian, etc.) quality. According to some non-limiting aspects, the deformable, conductive materials can include an electroactive material, such as a deformable conductors produced from a conductive gel (e.g., a gallium indium alloy). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring traces 104ₐ, 104_{b} of varying forms and/or compositions to achieve the benefits disclosed herein.

In some aspects, a viscosity of the deformable conductive material may, when under high shear (e.g., in motion), be in a range of about 10 Pascal seconds (Pa*s) and 500 Pa*s, such as a range of 50 Pa*s and 300 Pa*s, and/or may be about 50 Pa*s, about 60 Pa*s, about 70 Pa*s, about 80 Pa*s, about 90 Pa*s, about 100 Pa*s, about 110 Pa*s, about 120 Pa*s, about 130 Pa*s, about 140 Pa*s, about 150 Pa*s, about 160 Pa*s, about 170 Pa*s, about 180 Pa*s, about 190 Pa*s, or about 200 Pa*s. In some aspects, a viscosity of the deformable conductive material may, when under low shear (e.g., at rest), be in a range of 100,000 Pa*s and 40,000,000 Pa*s, such as a range of 1,000,000 Pa*s and 40,000,000 Pa*s, and/or may be about 10,000,000 Pa*s, about 20,000,000Pa*s, about 30,000,000 Pa*s, or about 40,000,000 Pa*s.

In further reference to FIG. 1, the first layup 102ₐ can be configured to mechanically interface the second layup 102_{b} such that a seal 108 can be formed between the first layup 102ₐ and the second layup 102_{b}. The seal 108 can be formed via a process configured to attach an outer perimeter of the first layup 102ₐ to an outer perimeter of the second layup 102_{b}, including any known process that uses heat, pressure, radio-frequency energy, and/or additional materials for attachment (e.g., welding, soldering, fusing, stitching, adhesives, etc.). According to some non-limiting aspects, the seal 108 can be formed using a radio-frequency weld procedure, an adhesive (e.g., a thermoset, a thermoplastic adhesive film, etc.), or a thermos-sensitive mask coating (e.g., silicone) that can be applied to regions of the layups 102ₐ, 102_{b} that will not be scaled (e.g., inner surfaces of the cavity). As such, an inner surface 113ₐ (FIG. 2) of the first layup 102ₐ and an inner surface 113_{b} (FIG. 2) of the second layup 102_{b} can collectively define an internal cavity 110 (FIG. 2) configured to accommodate a fluid (e.g., compressible, non-compressible). Because the first layup 102ₐ, the second layup 102_{b}, and the traces 104ₐ, 104_{b} of the circuit 100 may be fabricated from flexible materials, the circuit 100 of FIG. I can stretch as the fluid is introduced to the internal cavity. In other words, the circuit 100 of FIG. I can be selectively inflated and deflated as the fluid is introduced and/or removed from the internal cavity. According to the non-limiting aspect of FIG. 1, the fluid-fillable circuit 100 is depicted in an inflated condition, meaning the internal cavity defined by an inner surface 113ₐ (FIG. 2) of the first layup 102ₐ and an inner surface 113_{b} (FIG. 2) of the second layup 102_{b} is accommodating a fluid.

Still referring to FIG. 1, the circuit 100 can be inflated using various known methods of inflation, such as the method described in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety. For example, the first layup 102ₐ and the second layup 102_{b} of FIG. 1 can include a multi-layer construction, and at least a portion of the first layup 102ₐ and the second layup 102_{b} can be fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 100 of FIG. 1, and fluid can be selectively introduced and/or removed from the internal cavity via the valve assembly. For example, according to some non-limiting aspects, the circuit 100 can include a valve assembly similar to those disclosed in U.S. Pat. No. 5,257,470, titled SHOE BLADDER SYSTEM, and issued on Nov. 2, 1993, the disclosure of which is hereby incorporated by reference in its entirety.

Referring now to FIG. 2, a cross-sectioned view of either the fluid-fillable circuit 100 of FIG. 1 is depicted in accordance with at least one non-limiting aspect of the present disclosure. The cross-section of FIG. 2 was taken about line 1B in FIG. 1. In FIG. 2, the multi-layer nature of the first layup 102ₐ and the second layup 102_{b} becomes apparent. For example, according to the non-limiting aspect of FIG. 2, the layups 102ₐ, 1012_{b} can include a two-layer 112, 114 construction. Specifically, each of the first layup 102ₐ and the second layup 102_{b} of the fluid-fillable circuit 100 of FIG. 1 can include a substrate layer 112ₐ, 112_{b} and an encapsulation layer 114ₐ, 114_{b}. However, according to other non-limiting aspects, the layups 102ₐ, 1012_{b} can include three or more layers, including a stencil layer configured to accommodate the traces 104_{a,b} (FIG. 1), as described in U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. In still other non-limiting aspects, the layups 102ₐ, 102_{b} can include a single layer configured to accommodate the traces 104ₐ, 104_{b}.

Alternately and/or additionally, the flexible structures described in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety, can be used to fabricate one or more layers of the layups 102ₐ, 102_{b}, as depicted in FIG. 2. For example, the layups 102ₐ, 102_{b} can be produced from one or more layers including a B-stage resin film, a C-stage resin film, an adhesive, a TPU, and/or a silicone material, amongst others.

According to the non-limiting aspect of FIG. 2, the substrate layers 112ₐ, 112_{b} of the first and second layups 102ₐ, 102_{b} can include one or more features, such as a contact point configured to mechanically and/or electrically engage with an electronic component mounted to the layups 102ₐ, 102_{b} and/or one or more of the traces 104_{a,b}. The traces 104_{a,b} and, more specifically, a deformable conductor from which the traces 104_{a,b} are composed, can be deposited either on or embedded within a portion of the substrate layers 112ₐ, 112_{b}. Collectively, the encapsulation layers 114ₐ, 114_{b} can contain and protect the fluid-fillable circuit 100, including any traces 106, electronic components, and/or contact points coupled to the substrate layers 112ₐ, 112_{b}. The encapsulation layers 114ₐ, 114_{b} can also fill any spaces between the components and the substrate layers 112ₐ, 112_{b}. For example the encapsulation layer 114 can be formed from materials suitable for the encapsulation of electronics, including silicone-based materials such as PDMS, urethanes, epoxies, polyesters, polyamides, and/or varnishes, amongst other materials capable of providing a sufficient protective coating and/or assisting in holding the fluid-fillable circuit 100 assembly together.

According to some non-limiting aspects, the circuit 100 of FIG. 2 can be assembled in accordance with the design for manufacture techniques disclosed in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety. For example, the traces 104ₐ, 104_{b}, vias 106ₐ, 106_{b}, and contacts (not shown) may be particularly sized and spaced, the ampacity of traces 104ₐ, 104_{b} may be configured, and the various features of the layups 102ₐ, 102_{b} may be attached in accordance with the techniques described for non-inflatable laminate structures, as disclosed therein.

For example, the substrate layers 112ₐ, 112_{b} and encapsulation layers 114ₐ, 114_{b} can be configured similar to the substrate layers and encapsulants described in U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. Alternatively and/or additionally, the substrate layers 112ₐ, 112_{b} and encapsulation layers 114ₐ, 114_{b} can be formed from a microlayer membrane that facilitates inflation of the fluid-fillable circuit 100 with a compressible fluid, such as the membranes disclosed in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety.

As depicted in FIG. 2, one or more vias 106 can be configured to traverse at least a portion of the substrate layers 112ₐ, 112_{b} and/or the encapsulation layers 114ₐ, 114_{b} of the layups 102ₐ, 102_{b}, creating an electrical conduit by which a desired electrical connection between electrical features of the first and second layups 102ₐ, 102_{b} can be established. For example, according to the non-limiting aspect of FIG. 2, the vias 106 can be formed through the substrate layers 112ₐ, 112_{b} such that the traces 104ₐ, 104_{b} of the first and second layups 102ₐ, 102_{b} are electrically coupled. However, vias 106 can similarly be configured to electrically couple electronic components and/or contact points coupled to each of the first and second layups 102ₐ, 102_{b}. According to other non-limiting aspects, the top layup 102ₐ and the bottom layup 102_{b} can each be independently encapsulated, such that each surface of the substrate layer 112ₐ of the top layup 102ₐ and the substrate layer 112ₐ of the bottom layup 102_{b} is covered by the encapsulation layer 114ₐ of the top layup 102ₐ and the encapsulation layer 114_{b} of the bottom layup 102_{b}, respectively. In such aspects, one or more vias 106 may traverse the encapsulation layers 114ₐ, 114_{b}, such that a trace 106ₐ on the substrate layer 112ₐ of the top layup 112ₐ is electrically coupled to a trace 106_{b} on the substrate layer 112_{b} of the bottom layup 102_{b}.

According to other non-limiting aspects, the layups 102ₐ, 102_{b} can further include a stencil configured to accommodate the traces 104ₐ, 104_{b}, such as those described in U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. For example, a stencil can be particularly constructed to define paths for one or more of the traces 104ₐ, 104_{b} that traverse the fluid-fillable circuit 100 in accordance with a schematic for the fluid-fillable circuit 100. The paths defined by such stencils can accommodate the deformable conductor, which can be deposited within the stencil and covered by the encapsulation layers 114ₐ, 114_{b} such that the stencil and deformable conductors, which define the traces 104ₐ, 104_{b}, are bound between the substrate layers 112ₐ, 112_{b} and encapsulation layers 114ₐ, 114_{b}. Optionally, a pattern of traces may be formed in or on the substrate layers 112ₐ, 112_{b}, e.g., by laser ablation, thermoforming, molding, or other suitable additive or subtractive methods prior to depositing the deformable conductor on or in the substrate layer.

Still referring to FIG. 2, the internal cavity 110 collectively defined by the inner surface 113ₐ of the first layup 102ₐ, the inner surface 113_{b} of the second layup 102_{b}, and the seal 108 is illustrated in accordance with at least one non-limiting aspect of the present disclosure. As noted in reference to FIG. 1, the internal cavity 110 can accommodate a fluid (e.g., octafluoropropane, nitrogen, air, etc.), which can be compressible or non-compressible. The fluid can be pressurized as required by the particular application. For example, it may be desirable to pressurize the internal cavity 110 to a gauge pressure within a range of 1-35 pounds-per-square-inch (“PSI”). According to some non-limiting aspects, the internal cavity 110 may be configured to accommodate a pressure of about 20 PSI in an inflated condition. In other words, the fluid-fillable circuit 100—and more specifically, the internal cavity 110 collectively defined by the inner surface 113ₐ of the first layup 102ₐ, the inner surface 113_{b} of the second layup 102_{b}, and the seal 108—can be particularly configured to accommodate an internal fluid pressure that is slightly above ambient, or relatively high. In still other non-limiting aspects, the fluid-fillable circuit 100 can be similarly configured to bladders made using aspects described in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety.

As depicted in FIG. 2, the fluid-fillable circuit 100 can be geometrically configured such that the internal cavity 110 includes a stadium-like (e.g., substantially rectangular, with rounded edges) cross section. However, the fluid-fillable circuit 100 can be alternately configured to have a number of different geometries, depending on user preference and/or intended application. For example, according to some non-limiting aspects, the fluid-fillable circuit 100 can be circular, spherical, hexagonal, rectangular, triangular or irregularly shaped. Such configurations may not be apparent from the cross-section of the fluid-fillable circuit 100, but evident when the fluid-fillable circuit 100 is viewed from above. In still other non-limiting aspects, the fluid-fillable circuit 100 can have an arbitrary or abstract geometry, such as the bladders described in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety. Such bladders are generally hexagonal but include one or more indents that abstract the geometry. According to other non-limiting aspects, the fluid-fillable circuit 100 can be one of a plurality of inflatable circuits 100, wherein the plurality of inflatable circuits 100 are arranged in a tessellated pattern. In other words, each fluid fillable circuit 100 of the plurality can have a substantially similar shape and can be arranged such that the plurality of inflatable circuits 100 collectively cover an area without a significant gap and/or overlap.

Alternately and/or additionally, a fluid-fillable circuit 100 may be formed such that the circuit 100 is substantially flat in an uninflated configuration but can be molded to have varied three-dimensional surface topographies and shapes with varying degrees of complexity and contour. According to such aspects, three-dimensional forming can occur at the time of inflation, for example by thermoforming in a mold cavity having the desired final three-dimensional shape using, for example, inflation pressure. According to other non-limiting aspects, a fluid-fillable circuit 100 or system may be similarly molded in a secondary operation post-inflation, for example by thermal compression-molding in a mold cavity having the desired final 3-dimensional shape. It shall be appreciated that such methods of formation can be employed to form any of the circuits disclosed herein.

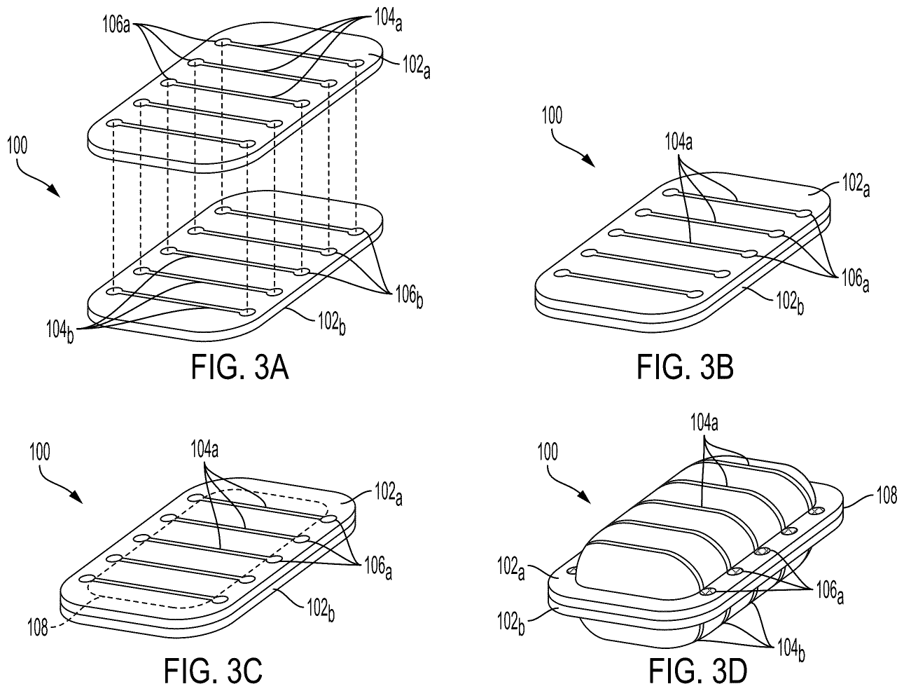

Referring now to FIGS. 3A-3D, several assembly diagrams of the fluid-fillable circuit 100 of FIG. 1 are depicted in accordance with at least one non-limiting aspect of the present disclosure. Specifically, FIGS. 3A-3D illustrate how the first layup 102ₐ can include features (e.g., traces 104ₐ, vias 106ₐ, etc.) that can be particularly dimensioned and positioned on the first layup 102ₐ, such that they correspond to features (e.g., traces 104_{b}, vias 106_{b}, etc.) on the second layup 102_{b}. For example, as depicted in FIG. 3A, the traces 104ₐ and vias 106ₐ of the first layup 102ₐ are dimensioned and positioned on the first layup 102ₐ such that they align with and electrically coupled to a corresponding trace 104_{b} or via 106_{b} on the second layup 102_{b}. This alignments is depicted in FIG. 3B, where the first layup 102ₐ is positioned above the second layup 102_{b}, such that corresponding features of the first and second layup 102ₐ, 102_{b} can be electrically coupled.

According to the non-limiting aspect of FIG. 3B, the first layup 102ₐ can have a design that is substantially similar (e.g., a one-for-one match) to a design of the second layup 102_{b}. In other words, the features of the first layup 102ₐ can be similarly dimensioned and positioned relative to corresponding features of the second layup 102_{b}. However, according to other non-limiting aspects, the first layup 102ₐ can be alternately designed relative to the second layup 102_{b}. Some or all of the features (e.g., traces 104ₐ, vias 106ₐ, etc.) of the first layup 102ₐ can be alternately dimensioned and/or positioned relative to corresponding features on the second layup 102_{b}. For example, it might be desirable to account for manufacturing tolerances and/or alignment issues by dimensioning one or more of the vias 106ₐ of the first layup 102ₐ with a larger diameter relative to a diameter of a corresponding via 106_{b} of the second layup 102_{b}. Alternately and/or additionally, one or more of the vias 106ₐ of the first layup 102ₐ can be positioned such that it is staggered relative to a corresponding via 106_{b} of the second layup 102_{b}. Such design modifications can ensure a proper electrical connection between traces 104ₐ, 104_{b} can be achieved. In still other non-limiting aspects, the features of the layups 102ₐ, 102_{b} have different geometric configurations (e.g., triangular, rectangular, hexagonal, three-dimensional, etc.).

According to still other non-limiting aspects, the layups 102ₐ, 102_{b} of FIGS. 3A-3D can have differing sizes and/or shapes. For example, according to some non-limiting aspects, the second layup 102_{b} can have a larger overall surface area relative to the first layup 102ₐ, or vice-versa, such that only a portion of the circuit 100 is inflatable and some features (e.g., traces 104_{b}, vias 106_{b}, etc.) of the second layup 102_{b} are do not correspond to the shape of the internal cavity 110 (FIG. 2). Layups 102ₐ, 102_{b} of differing sizes and/or shapes can be useful for aspects in which the fluid-fillable circuit 100 is integrated into an external structure or housing, wherein only a portion of the structure or housing is intended to support inflation of the circuit 100, but where a stretchable, bendable, or otherwise flexible circuit is integrated to other portions.

Additionally and/or alternately, such non-inflated circuit portions can be in electrical communication with inflated portions of the fluid-fillable circuit 100. Furthermore, both the first and second layups 102ₐ, 102_{b} can have portions that do not correspond to the shape (e.g., when viewed in plan, for example) of the internal cavity 110, and in some non-limiting aspects, such portions may overlap and/or substantially overlay one another. According to still other non-limiting aspects overlapping or overlaid portions can be bonded or otherwise unitized to one another. In all such examples where at least one uninflated portion does not correspond to the shape of the internal cavity is provided in either and/or both of the first and second layups 102ₐ, 102_{b}, such uninflated portions may extend from a surface of the inflated portion of the circuit 100 and/or the seal 108, itself. Such uninflated portions may extend into the cavity 110 so as to be contained within the internal volume of the inflated circuit, or they may extend away from the inflated portion such that they are external to the volume of the cavity 100. It shall be appreciated that similar uninflated portions can be implemented in any of the circuits disclosed herein.

As such, it shall be appreciated that the geometric configuration of the layups 102ₐ, 102_{b} of FIGS. 3A-3D, including the geometric configuration of their respective features (e.g., traces 104ₐ, 104_{b}, vias 106ₐ, 106_{b}, etc.) are not intended to be limiting, and that the present disclosure contemplates numerous aspects wherein the geometric configuration of the layups 102ₐ, 102_{b} are attenuated to achieve a desired mechanical and/or electrical integration of the fluid-fillable circuit 100.

According to FIG. 3C, once the layups 102ₐ, 102_{b} are aligned, as depicted in FIG. 3B, features (e.g., traces 104ₐ, vias 106ₐ, etc.) of the first layup 102ₐ can be electrically coupled to corresponding features (e.g., traces 104_{b}, vias 106_{b}, etc.) of the second layup 102_{b}. For example, in FIG. 3C, the substrate layer 112ₐ of the top layup 102ₐ can be brought into contact with or otherwise positioned adjacent the substrate layer 112_{b} of the bottom layup 102_{b}, such that the vias align 106ₐ, 106_{b} and corresponding traces 104ₐ of the top layup 102ₐ are in electrical communication with the traces 104_{b} of the bottom layup 102_{b}. It shall be appreciated that in embodiments having layups that are mirror images of one another, such as the ones depicted here, it may be desirable that corresponding features such as vias are formed in the same layer of both layups (e.g., the encapsulation layers, or the substrate layers) for ease of manufacturing and inventory control. However, in embodiments (not shown here) where the various layups are not mirror images of one another, it may be preferable but not necessary for corresponding features such as vias to be formed in different layers of each layup (e.g., the encapsulation layer of one and the substrate layer of the other). A region of the first layup 102ₐ can be subsequently bonded or unitized to a corresponding region of the second layup 102_{b} by any known process of attachment (e.g., welding, soldering, fusing, stitching, adhesives, etc.), thereby creating the seal 108 of the internal cavity 110 (FIG. 2). According to the non-limiting aspect of FIG. 3C, the corresponding regions—and thus, the sealed portion—of the first and second layups 102ₐ, 102_{b} can be located on the perimeter of the first and second layups 102ₐ, 102_{b}, and can include any overlapping vias 106ₐ, 106_{b} positioned in those regions. As such, the first and second layups 102ₐ, 102_{b} can be securely fastened, and the traces 104ₐ, 104_{b} reliably held in electrical communication by the aligned vias 106ₐ, 106_{b}. Accordingly, the fluid-fillable circuit 100 can be assembled such that a portion of the inner surface 113ₐ (FIG. 2) of the first layup 102ₐ is not adhered to a portion of the inner surface 113_{b} (FIG. 2) of the second layup 102_{b} and thus, the portion of the inner surface 113ₐ (FIG. 2) can be separated from the portion of the inner surface 113_{b} (FIG. 2). Such separation can be caused by a fluid inserted into the internal cavity 110 (FIG. 2) and contained within the internal cavity 110 (FIG. 2) via the seal 108. Thus, due to the deformable nature of the substrate layers 112ₐ, 112_{b} (FIG. 2), encapsulation layers 114ₐ, 114_{b} (FIG. 2), and traces 104ₐ, 104_{b}, the circuit 100 can be inflated, as illustrated in FIG. 3D. It may be appreciated that after sealing, portions of the seal 108 may not be structurally or electrically necessary and may be trimmed from the fluid-fillable circuit 100 for aesthetic or other reasons and that this is true for all of the circuits disclosed herein.

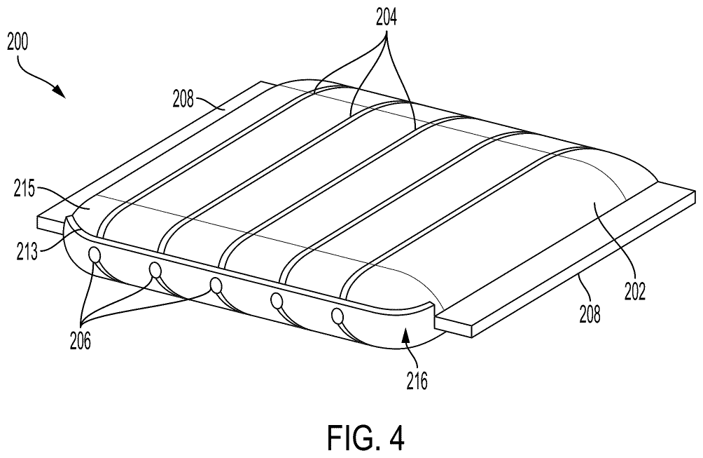

Referring now to FIG. 4, a perspective view of another inflatable circuit 200 is depicted in accordance with at least one non-limiting aspect of the present disclosure. According to the non-limiting aspect of FIG. 4, the fluid-fillable circuit 200 can include a single layup 202 with a plurality of traces 204. The single layup 202 of FIG. 4 can include a multi-layer construction, similar to the layups 102ₐ, 102_{b} of FIG. 2. Likewise, each trace 204 of the plurality can be constructed from a deformable conductive material deposited on or within a substrate layer and encapsulated. According to some non-limiting aspects, the layup 202 can further include a stencil, which can define paths for the traces 204 in which the deformable conductor can be deposited and contained.

According to the non-limiting aspect of FIG. 4, the layup 202 can be folded or rolled such that a first portion of the layup 202 overlaps with a corresponding, second portion of the layup 202. The layup 202 can be geometrically configured such that, when the layup 202 is folded, one or more vias 206 positioned on the first portion of the layup 202 can be aligned and placed in electrical communication with a corresponding via 206 the second portion of the layup 202. The alignment of the vias 206 in this embodiment results in a desired alignment of traces 204, which traverse between a via 204 on the first portion and a via 206 on the second portion. In some examples, traces 204 may have patterns that do not overlay as shown. Once the vias 206 and traces 204 are aligned, the first portion of the layup 202 can be bonded to the second portion of the layup 202 by any known process of attachment (e.g., welding, soldering, fusing, stitching, adhesives, etc.), thereby creating a sealed lap joint 216 between an inner surface 213 and an outer surface 215 of the layup 202. According to the non-limiting aspect where the single layup 202 has a construction similar to the layups 102ₐ, 102_{b} of FIG. 2, an encapsulation layer of the layup 202 can overlap and be bonded to a substrate layer of the layup 202 to form the lap joint 216. The creation of the lap joint 216 may result in non-overlapping, side portions of the layup 202, which may be bonded via any known process of attachment (e.g., welding, soldering, fusing, stitching, adhesives, etc.), thereby creating a seal 208 on either side of the fluid-fillable circuit 200. The seal 208 can be formed in the same bonding operation as the lap joint 216 or, alternately, via a separate bonding operation. According to some non-limiting aspects, the layup 202 can be alternately configured such that the otherwise additional lap joints can be formed on either side of the fluid-fillable circuit 200, such that the additional lap joints can be used in lieu of the seals 208 of FIG. 4.

Similar to the multi-layup circuit 100 of FIG. 2, when assembled, the circuit 200 of FIG. 4 can define an internal cavity configured to accommodate and contain a fluid. Specifically, the internal cavity can be defined by the inner surface 213, the lap joint 216, and the seal 208 of the layup 202. Although the circuit 200 of FIG. 4 defines an internal cavity having a stadium-like cross section, the fluid-fillable circuit 200 can be alternately configured to have a number of different geometries, depending on user preference and/or intended application. For example, according to some non-limiting aspects, the fluid-fillable circuit 200 can be circular, hexagonal, rectangular, or triangular. Similar to the circuit 100 of FIG. 1, according to other non-limiting aspects, the circuit 200 of FIG. 4 is one of a tessellated plurality. Regardless of its particular geometric configuration, the circuit 200 of FIG. 4 can be inflated by inserting a fluid into the internal cavity defined by the inner surface 213, the lap joint 216, and the seal 208. Notably, FIG. 4 depicts the circuit 200 in an inflated condition.

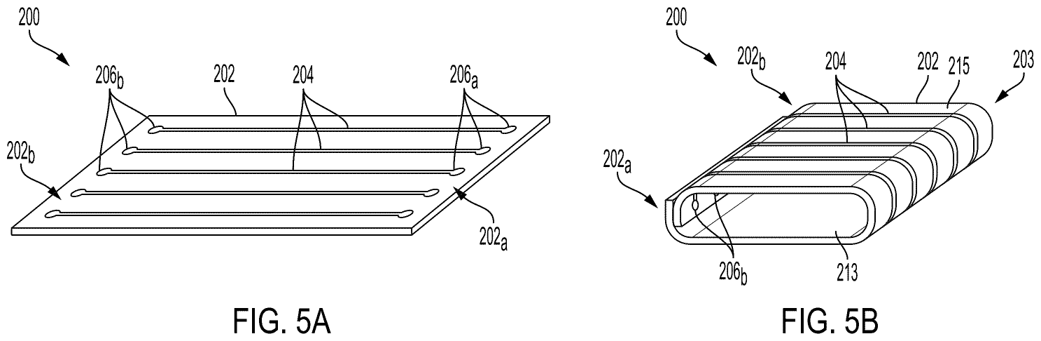

Referring now to FIGS. 5A-5D, several assembly diagrams of the fluid-fillable circuit 200 of FIG. 4 are depicted in accordance with at least one non-limiting aspect of the present disclosure. Specifically, FIG. 5A illustrates how the layup 202 can have a first portion 202ₐ and a second portion 202_{b}. A first plurality of vias 206ₐ can be positioned on the first portion 202ₐ and a second plurality of vias 206_{b} can be positioned on the second portion 202_{b}. Each trace 204 from the plurality of traces 204 can be electrically coupled to corresponding vias 206ₐ, 206_{b} and thus, configured to traverse the layup 202 from the first portion 202ₐ to the second portion 202_{b}.

In reference to FIG. 5B, a fold 203 in the layup 202 is illustrated such that the first portion 202ₐ of the layup 202 is aligned with the second portion 202_{b}. More specifically, the vias 206ₐ (not shown) of the first portion 202ₐ are aligned with the vias 206_{b} of the second portion 202_{b}. After alignment, the vias 206ₐ (not shown) of the first portion 202ₐ can be electrically coupled to the vias 206_{b} of the second portion 202_{b}. Moreover, the alignment of FIG. 5B prepares the layup 202 for the bonding procedure that will result in the lap joint 216. For example, the fold 203 in the layup 202 is particularly configured such that an inner surface 213 of the first portion 202ₐ of the layup 202 can mechanically engage an outer surface 215 of the second portion 2026 of the layup 202.

Accordingly, the layup 202 of FIG. 5B is properly aligned and prepared for the bonding process, as depicted in FIG. 5C. The first portion 202ₐ of the layup 202 can be bonded to the second portion 202_{b} of the layup 202 by any known process of attachment (e.g., welding, soldering, fusing, stitching, adhesives, etc.), thereby creating the lap joint 216. The process can further secure the electrical connection of the vias 206ₐ on the first portion 202ₐ of the layup 202 to the vias 206_{b} on the second portion 202_{b} of the layup 202. Since, each trace 204 is electrically coupled to corresponding vias 206ₐ, 206_{b}, when each pair of corresponding vias 206ₐ, 206_{b} are electrically coupled, that part of the circuit 200 (e.g., first via 206ₐ, second via 206_{b}, and the connecting trace 204) is closed and mechanically secured to ensure a robust electrical connection.

Referring now to FIG. 5D, after the lap joint 216 is formed, the circuit 200 can be flattened along the fold 203, such that a majority of the inner surface 213 of the layup 202 is faces, but is not adhered to another portion of the inner surface 213 of the layup 200. Accordingly, a seal 208 may be formed on either side of the circuit, wherein each seal 208 is perpendicular to the lap joint 216. The seals 208 can be formed by any known process of attachment (e.g., welding, soldering, fusing, stitching, adhesives, etc.) and this process can be done in conjunction with, or separate from, the creation of the lap joint 216. One or more notches can be introduced into the layup 202, prior to the formation of the seals 208, to reduce mechanical interference during inflation.

Accordingly, the fluid-fillable circuit 200 can be assembled such that a majority of the inner surface 213 of the layup 202 is folded such that it faces—but is not adhered to—another portion of the inner surface 213 of the layup 200 and thus, forms an internal cavity of the fluid-fillable circuit 200. A fluid can be inserted into the internal cavity formed by the inner surface 213, lap joint 216, and seals 208 of the layup 200 and contained. As more fluid is inserted within the internal cavity, the more pressure will be exerted on the inner surfaces 213 of the layup 202. Due to the deformable nature of the layup 202 and traces 202, the circuit 200 can be inflated, as illustrated in FIG. 4.

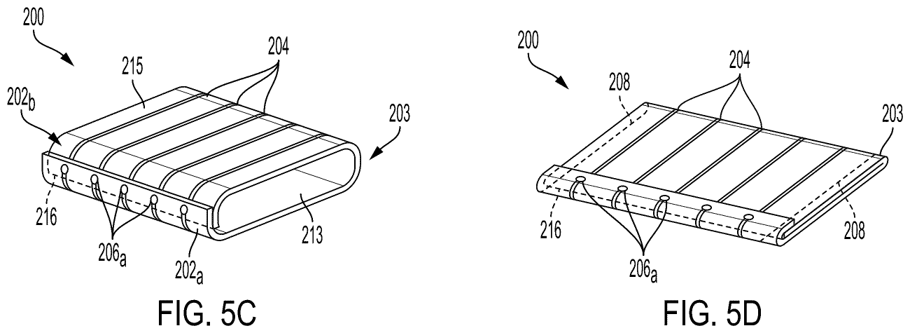

Referring now to FIG. 6, a perspective view of another inflatable circuit 300 is depicted in accordance with at least one non-limiting aspect of the present disclosure. Similar to the circuit 200 of FIG. 4, the circuit 300 of FIG. 6 can include a single layup 302 construction, wherein the single layup 302 can include a plurality of traces 304 and a plurality of vias 306. Once again, the single layup 302 of FIG. 6 can include a multi-layer construction, similar to the layups 102ₐ, 102_{b} of FIG. 2. Likewise, each trace 304 of the plurality can be constructed from a deformable conductive material deposited on or within a substrate layer and encapsulated. According to some non-limiting aspects, the layup 302 can further include a stencil layer, which can define paths for the traces 304 in which the deformable conductor can be deposited and contained.

Similar to the circuit 200 of FIG. 4, the layup 302 can be folded such that a first portion of the layup 302 interfaces with a corresponding, second portion of the layup 302. The layup 302 can be geometrically configured such that, when the layup 302 is folded or rolled, one or more vias 306 positioned on the first portion of the layup 302 can be aligned and placed in electrical communication with a corresponding via 306 the second portion of the layup 302. The alignment of the vias 306 may result in a desired alignment of traces 304 as shown in this embodiment, or traces may be misaligned or otherwise have an asymmetric configuration. As shown here, the vias traverse linearly and in alignment between a via 304 on the first portion and a via 304 on the second portion. However, according to the non-limiting aspect of FIG. 6, the circuit 300 can be folded or rolled such that every portion of an inner surface 313 of the layup 302 faces another portion of the inner surface 313 of the layup. In other words, no portion of the layup 302 of FIG. 6 overlaps with another portion and thus, the circuit 300 of FIG. 6 does not have a lap joint 216 (FIG. 4), such as the circuit 200 of FIG. 4. Rather, once the vias 306 and traces 304 are aligned, a seal 308 can be formed between inner surfaces 313 of the first and second portions of the layup 302 by any known process of attachment (e.g., welding, soldering, fusing, stitching, adhesives, etc.). In an alternative embodiment (not shown), the outer surface 315 may be folded or rolled over at a free edge of the layup 302, e.g., the edge comprising vias 306, such that the outer surfaces 315 contact one another, and the seal 308 formed adjacent this edge such that the seal 308 is contained within the cavity defined within the interior surface 313 of the layup 302. The remaining edges, e.g., the remaining two edges at opposite ends of the circuit 300, may be sealed as shown here. In another aspect (not shown), two free edges may be folded or rolled over such that the outer surfaces may contact one another, and the seal 308 formed adjacent these edges such that the seal is contained between the interior surface 313. The remaining edge(s), e.g., the remaining edges comprising the vias of the circuit 300, may be sealed as shown here. According to the non-limiting aspect where the single layup 302 has a construction similar to the layups 102ₐ, 102_{b} of FIG. 2, an encapsulation or substrate layer of the layup 302 can be overlaid onto itself such that vias 306 positioned on the first portion of the layer can be aligned with and electrically coupled to a corresponding via 306 positioned on the second portion of the encapsulation layer. The seal(s) 308 may comprise the overlaid vias, thereby providing reliable mechanical and electrical coupling of the vias to one another.

According to the non-limiting aspect of FIG. 6, the seal 308 can create an edge joint 316 that, along with the fold 303, can form an internal cavity similar to the internal cavity 110 of FIG. 2. Similar to the multi-layup circuit 100 of FIG. 2, when assembled, the internal cavity defined by the circuit 300 of FIG. 6 can accommodate and contain a fluid. Although the circuit 300 of FIG. 6 defines an internal cavity having a stadium-like cross section, the fluid-fillable circuit 300 can be alternately configured to have a number of different geometries, depending on user preference and/or intended application. For example, according to some non-limiting aspects, the fluid-fillable circuit 300 can be circular, hexagonal, rectangular, or triangular. Similar to the circuit 100 of FIG. 1, according to other non-limiting aspects, the circuit 300 of FIG. 6 is one of a tessellated plurality. Regardless of its particular geometric configuration, the circuit 300 of FIG. 6 can be inflated by inserting a fluid into the internal cavity defined by the inner surface 313, the edge joint 316, and the fold 303. Notably, FIG. 6 depicts the circuit 300 in an inflated condition. According to other non-limiting aspects, a butt joint can be utilized to seal the circuit 300 about a thickness of the layup 302, in lieu of the edge joint 316 of FIG. 6.

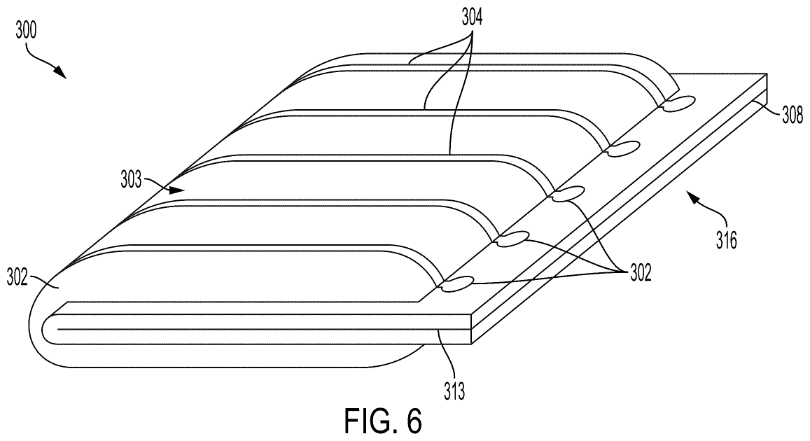

Referring now to FIGS. 7A-7D, an assembly of the fluid-fillable circuit 300 of FIG. 6 is depicted in accordance with at least one non-limiting aspect of the present disclosure. Specifically, FIG. 7A illustrates how the layup 302 can have a first portion 302ₐ and a second portion 302_{b}. A first plurality of vias 306ₐ can be positioned on the first portion 302ₐ and a second plurality of vias 306_{b} can be positioned on the second portion 302_{b}. Each trace 304 from the plurality of traces 304 can be electrically coupled to corresponding vias 306ₐ, 306_{b} and thus, configured to traverse the layup 302 from the first portion 302ₐ to the second portion 302_{b}.

In reference to FIG. 7B, a fold 303 in the layup 302 is illustrated such that the first portion 302ₐ of the layup 302 is aligned with the second portion 302_{b}. According to the non-limiting aspect of FIG. 7B, the circuit 300 can be folded such that the edges of the portions 302ₐ and 302_{b} are aligned. As shown in this aspect, the inner surface 313 of the first portion 302ₐ of the layup 302 faces the inner surface 313 of the second portion 302_{b} of the layup 302. The vias 306ₐ of the first portion 302ₐ of the layup 302 can be aligned with the vias 306_{b} (not shown) of the second portion 302_{b} of the layup 302. After alignment, the vias 306ₐ of the first portion 302ₐ can be electrically coupled to the vias 306_{b} (not shown) of the second portion 302_{b}. Moreover, the alignment of FIG. 7B prepares the layup 302 for the bonding procedure that will result in an edge joint 316.

Accordingly, the layup 302 of FIG. 7B is properly aligned and prepared for the bonding process, as depicted in FIG. 7C. A seal 308 can be formed between the first portion 302ₐ and the second portion 302_{b}, as shown here at the inner surface 313, by any known process of attachment or unitizing (e.g., welding, soldering, fusing, stitching, adhesives, etc.). The seal 308 of this embodiment creates an edge joint 316 between the first and second portions 302ₐ, 302_{b} that comprises the vias 306ₐ, 306_{b}. The process as shown may thus mechanically secure the electrical connection of the vias 306ₐ to the vias 306_{b}. Since each trace 304 is electrically coupled at corresponding vias 306ₐ, 306_{b}, when each pair of corresponding vias 306ₐ, 306_{b} are electrically coupled that part of the circuit 300 (e.g., first via 306ₐ, second via 306_{b}, and the connecting trace 304) is closed and mechanically secured to ensure a robust electrical connection. It should be appreciated that a seal may be provided in a portion (not shown) of the circuit 300 that does not contain vias, and optionally the seal may not be located at free edges of the circuit 300 thereby providing uninflated portions (not shown) that extend beyond the seal. The seal may comprise traces and thus, traces from an inflated portion may be in electrical communication with uninflated portions of the circuit 300. It may be appreciated that vias may be located in any permutation of inflated portions, seals or joints, or uninflated portions of the circuit 300. Vias that connect one trace end to another trace end may provide open or closed electrical communication in a single trace or between different traces of the circuit 300, to an electronic component, or to another inflated or uninflated circuit.

In further reference to FIG. 7C, after the edge joint 316 is formed, a substantial portion of the inner surface 313 of the layup 302 can face—but is not adhered to—another portion of the inner surface 313 of the layup 300. Accordingly, the seal 308, which can be formed on every side of the circuit 300 that is not bound by the fold 303, can form an internal cavity (e.g., similar to the internal cavity 110 of FIG. 2) of the fluid-fillable circuit 300. A fluid can be inserted into and contained by the internal cavity formed by the inner surface 313, edge joint 316, and fold 303. As more fluid is inserted within the internal cavity, the more pressure will be exerted on the inner surfaces 313 of the layup 302. Due to the deformable nature of the layup 302 and traces 304, the circuit 300 can be inflated, as illustrated in FIG. 7D.

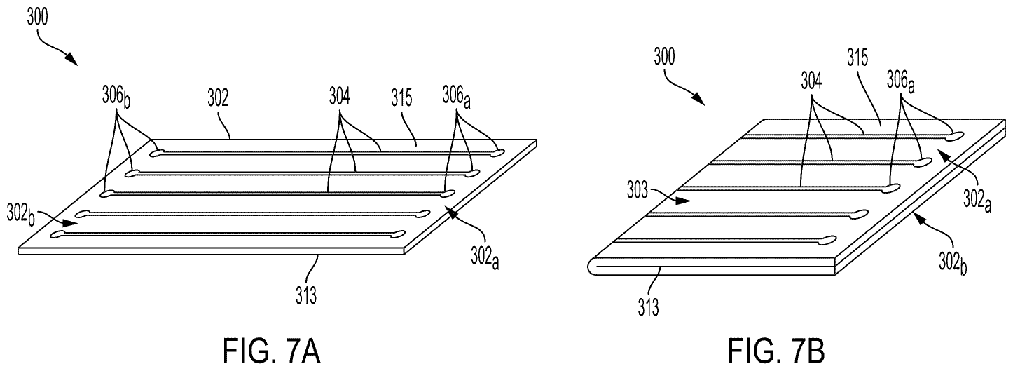

Referring now to FIG. 8, a perspective view of another inflatable circuit 400 is depicted in accordance with at least one non-limiting aspect of the present disclosure. According to the non-limiting aspect of FIG. 8, the traces 404ₐ, 404_{b} of the fluid-fillable circuit 400 can be particularly configured such that the fluid-fillable circuit can perform a variety of electrical functions. In other words, whereas the previously discussed aspects of FIGS. 1-7 described several non-limiting mechanical constructions and interconnects between traces of several inflatable circuit embodiments, the non-limiting aspect of FIG. 8 demonstrates a fluid-fillable circuit 400 electrically configured to perform an intended function, which can vary depending on the intended application.

Specifically, an electrical current and/or potential can be applied to the circuit 400, e.g., at vias 406 and, when the circuit 400 is inflated, an electrical parameter may be generated (e.g., an inductance, a resistance, a voltage drop, a capacitance, signal, an electromagnetic field, etc.) that is associated with the trace pattern configuration and applied current and/or potential.

According to some non-limiting aspects, electronic components (e.g., a power source, a microprocessor, a logic-based controller, a transceiver, an electrode, a light emitting diode (“LED”) bank, a pump etc.) can be mechanically secured to and/or otherwise integrated with the layups 402ₐ, 402_{b} and/or electrically coupled to the traces 404ₐ, 404_{b} of the circuit 400. The electronic components can be configured to receive and utilize signals from the traces 404ₐ, 404_{b} and electrical parameters from the traces 404ₐ, 404_{b} in accordance with the intended application. The electrical parameters and signals may vary with a physical change to the fluid-filled circuit 400, for example the inflation pressure, volume of the cavity, application of an external stimulus and/or deformation of the circuit 400. The resulting changes to the electrical parameters may be monitored, transmitted, or otherwise utilized to dynamically or statically calculate, infer or otherwise determine one or more physical or structural characteristics or conditions of the circuit 400, and/or stimuli applied to the circuit 400.

According to the non-limiting aspect of FIG. 8, the fluid-fillable circuit 400 can include a first layup 402ₐ and a second layup 402_{b}, which can be positioned, coupled, and sealed together in accordance with the fluid-fillable circuit 100 of FIG. 1. Specifically, the first layup 402ₐ can be coupled to the second layup 402_{b} via a seal 408 between inner surfaces (not shown) of the first and second layup 402ₐ, 402_{b}, thereby forming an edge joint 416 between the two layups 402ₐ, 402_{b}. The first layup 402ₐ, the second layup 402_{b}, the seal 408, and the edge joint 416 can collectively define an internal cavity of the fluid-fillable circuit 400, similar to the internal cavity 110 of FIG. 2. The internal cavity can be configured to accommodate and contain a fluid. However, it shall be appreciated that the present disclosure contemplates other aspects in which other joints may be used to couple the first and second layup 402ₐ, 402_{b}. According to other aspects, the fluid-fillable circuit 400 of FIG. 8 can be made from a single layup using the techniques described in reference to FIGS. 4 and 6 and the associated descriptions thereof. Substrate layers of the first layup 40ₐ and second layup 402_{b} can have an outer surface 415ₐ, 415_{b} on which one or more traces 404ₐ, 404_{b} made from a deformable conductor can be deposited and encapsulated.

In further reference to FIG. 8, the traces 404ₐ can form a multi-layer or multi-level coil in a layup, e.g., the layup 402ₐ. The coiled trace 404ₐ can have a depth, meaning the coils descend towards the substrate layer of the layup 402ₐ, in some embodiments forming an Escher-like staircase configuration. The coil may be constructed using the principles disclosed in U.S. Provisional Application No. 63/270,589, titled FLEXIBLE THREE-DIMENSIONAL ELECTRONIC COMPONENT, filed Oct. 22, 2021 the disclosure of which is hereby incorporated by reference in its entirety. The trace 404ₐ can be specifically configured to generate a desired electrical parameter when an electric current and/or voltage is applied to the circuit 400. For example, since the trace 404. of FIG. 8 is arranged in a coiled configuration, a current may generate a particular inductive and/or electromagnetic response as it is applied to the circuit 400 and traverses the trace 404ₐ. For example, an electromagnetic field may be generated by a current applied to the coil. However, other electrical parameters, such as an electrical resistance and/or a voltage drop across the trace 404ₐ may be simultaneously or separately generated and/or monitored as a current and/or potential is applied to the circuit 400 and the current traverses the trace 404ₐ.

Still referring to FIG. 8, a conductive layer 418, may be provided such as the conductors described in U.S. patent application Ser. No. 17/192,725, titled DEFORMABLE INDUCTORS, filed Mar. 4, 2021 and/or PCT Patent Application No. PCT/US2021/071374, titled WEARABLE ARTICLE WITH FLEXIBLE INDUCTIVE PRESSURE SENSOR, filed Sep. 3, 2021, the disclosures of both which are hereby incorporated by reference in their entirety. The conductive layer 418 can be mechanically coupled to an outer surface 415, an inner surface, or laid up in between any of the layers of the second layup 402_{b}. The conductive layer 418 can be generally positioned opposite the inductive coil formed by the trace 404ₐ on or within the first layup 402ₐ and can define a surface area that correlates to or that is large enough to fully encompass, or bound, all of the coils formed by the trace 404ₐ. Collectively, the coiled trace 404ₐ and conductive layer 418 can function as an inductive sensor configured to utilize inductive responses generated by the coiled trace 404ₐ to determine a relative distance between the coiled trace 404ₐ on the first layup 402ₐ and the conductive layer 418 on the second layup 402_{b}. The conductive layer 418 can be alternately and/or additionally configured to serve other functions, such as those described in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety. For example, the conductive layer 418 can be configured to simultaneously serve as an electromagnetic shield. Likewise, the conductive layer 418 can be manufactured from a conductive film and configured for use as a high-power bus, as needed. In other non-limiting aspects, the coiled trace 404ₐ is positioned on the bottom layup 402_{b} and the conductive layer 418 is positioned on the top layup 402ₐ, as long as they are opposing each other and the conductive layer 418 is large enough to encompass all of the coils formed by the trace 404ₐ.

According to some non-limiting aspects, one or more electronic components (e.g., a power source, a microprocessor, a logic-based controller, a transceiver, an electrode, a LED bank, a pump etc.) can be coupled to the circuit 400 via a trace 404ₐ, 404_{b} positioned on one of the layups 402ₐ, 402_{b}. Alternately and/or additionally, one or more electronic components can be coupled to one or more traces 40ₐ., 406_{b} of the layups 402ₐ, 402_{b}. The electronic component can be configured to receive and process signals from the coiled trace 404ₐ and/or the conductive layer 418. For example, the electronic component can be a microprocessor configured to correlate an inductive response generated by the coiled trace 404ₐ to relative distance between the coiled trace 04ₐ and the conductive layer 418, as will be further explained below. According to other non-limiting aspects, the electronic component can be a capacitor configured to create a resistor, an inductor, and a capacitor circuit configured to sense proximity of the coiled trace 404ₐ to the conductive layer 418.

Because the first layup 402ₐ, the second layup 402_{b}, and the traces 404ₐ, 404_{b} can be fabricated from flexible and/or deformable materials, the circuit 400 of FIG. 8 can stretch as a fluid is introduced to the internal cavity. In other words, the circuit 400 of FIG. 4 can be selectively inflated and deflated as the fluid is introduced and/or removed from the internal cavity. As the circuit 400 is inflated and deflated, one or more dimensions (e.g., length, cross-sectional area, etc.) of the traces 404ₐ will change. According to some non-limiting aspects, one or more dimensions of the electrical features (e.g., traces 404ₐ, 404_{b}) of the layups 402ₐ, 402_{b} can change between a fraction of a percent and about thirty percent as the circuit 400 transitions between an inflated and deflated condition. As the dimensions of the electrical features (e.g., traces 404ₐ, 404_{b}) change, one or more electrical parameters (e.g., an inductance, a resistance, a voltage drop, a capacitance, and an electromagnetic field, etc.) generated by the applied current as it traverses the circuit 400 will subsequently change. In other words, the electrical parameters of the circuit 400 will change as the circuit 400 is inflated and deflated, even though the applied current and/or voltage remains constant.

Although the deformation of the traces 404ₐ, 404_{b} discussed above a response to an internal pressure applied by the fluid onto the inner surfaces 413ₐ, 413_{b} of the layups 402ₐ, 402_{b}, it shall be appreciated that additional deformations may result from external pressures applied on the outer surfaces 415ₐ, 415_{b} of the layups 402ₐ, 402_{b}, which would cause further deformation of the layups 402ₐ, 402_{b} and traces 404ₐ, 404_{b}. According to some non-limiting aspects, a baseline deformation and a resulting electrical parameter can be established for an inflated condition of the circuit 400. Accordingly, any additional deformations and/or further differences in the generated electrical parameter due to those deformations can be compared to the baseline and utilized to characterize subsequent structural parameters associated with the circuit 400 associated with applied external pressures. This can be useful to monitor and characterize an environment in which the circuit 400 and/or the use of the circuit as an airbag, bladder, and/or cushion.

For example, according to the non-limiting aspect of FIG. 8, the coiled trace 404ₐ may generate a first inductive response when the circuit 400 is deflated and a second inductive response when the circuit 400 is inflated. The electrical parameter (e.g., strength of the inductive response) is a function of the distance between the coiled trace 404, and the conductive layer 418. The conductive layer 418 can detect electrical parameters (e.g., inductance) generated by the coiled trace 404ₐ, and can send signals associated with detected electrical parameters to one or more electronic components via one or more connecting traces 404ₐ, 404_{b} and/or one or more vias 406ₐ, 406_{b}. For example, the electronic components can include a microprocessor configured to determine a difference between detected electrical parameters and correlate the determined difference to a structural parameter (e.g., a strain, a stress, a pressure, a dimension, etc.) associated with the fluid-fillable circuit 400. According to some non-limiting aspects, the correlation can be based on a particular electrical parameter value or a particular range of electrical parameter values, which would provide the user with a margin of error in case an electrical parameter value is not exactly replicated each time the circuit expands and/or contracts. In other words, the fluid-fillable circuit 400 can be configured such that an electrical characterization of the fluid-fillable circuit 400 can be used to characterize a physical condition of the fluid-fillable circuit 400. This can be useful in applications where the fluid-fillable circuit 400 is a bladder, or airbag of sorts, and the user wants to monitor and adjust the inflation for use as an adjustable cushion.

According to the non-limiting aspect of FIG. 8, the coiled trace 404, and conductive layer 418 of FIG. 4 can be implemented to determine, quantify and/or otherwise characterize a strain applied to the fluid-fillable circuit 400 by the fluid in the internal cavity. However, it shall be appreciated that the coiled configuration of the trace 404ₐ of FIG. 8 is merely presented for illustrative purposes. According to other non-limiting aspects, other sensors and/or sensing techniques can be implemented in lieu of either the coiled trace 404ₐ and/or the conductive layer 418 of FIG. 4. For example, radio-frequency identification (“RFID”) techniques, surface-acoustic-wave sensors (“SAW”), and/or hall-effect sensors can be implemented to achieve a similar result. According to other non-limiting aspects, the electrical parameter can include an electrical resistance generated by the current applied to the circuit 400. As the circuit 400 is inflated and deflated, a cross-sectional area and length of the traces 404ₐ, 404_{b} changes, resulting in a difference in electrical resistance through the circuit 400. This difference can be similarly correlated to a structural parameter (e.g., a strain, a stress, a pressure, a dimension, etc.) associated with the fluid-fillable circuit 400.

It shall be further appreciated that different geometries will be better suited for different applications, and that some geometries may be easier to manufacture. For example, the rectangular form of the coil defined by the trace 404ₐ on the first layup 402ₐ may be easier to manufacture than a curved-spiral trace 404ₐ and more conducive for lamination. Additionally, the relative dimensions of the coil formed by the trace 404ₐ and the conductive layer 418 can be selected in accordance with user preference and/or intended application. In some non-limiting aspects, the coiled trace 404ₐ can have a width and length no more than 20 to 30 millimeters, making the circuit 400 ideal for a seamless integration into a higher-level of assembly. Furthermore, the number of coils formed by the trace 404ₐ can be configured for a desired performance and can account for any constraints in any direction, such as a maximum depth of coil descent, which may relate to a total number of coil turns the laminate structure may accommodate. For example, the one-and-a-half coils formed by the trace 404ₐ of FIG. 8 was shown to generate an effective 75% gain in inductance for the same area as a single coil.

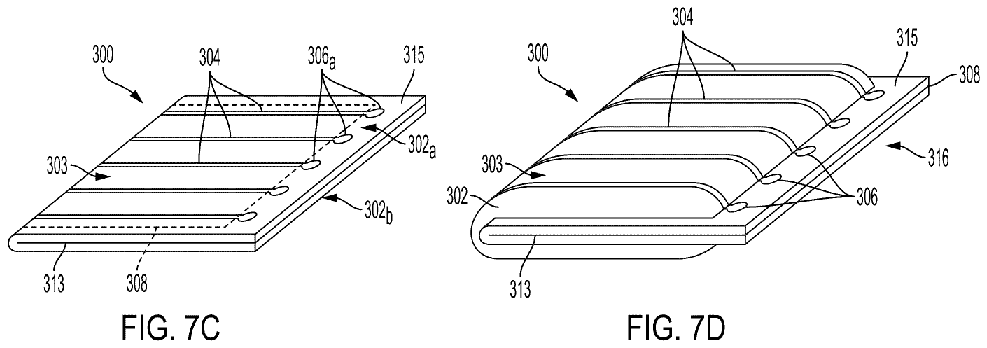

Referring now to FIGS. 9A-9D, an assembly of the fluid-fillable circuit 400 of FIG. 8 is depicted in accordance with at least one non-limiting aspect of the present disclosure. Specifically, FIG. 9A illustrates how the first layup 402ₐ can be aligned with the second layup 402_{b} such that the coiled trace 404ₐ aligns with the conductive layer 418. Moreover, the surface area of the conductive layer 418 is dimensioned such that the conductive layer 418 completely encompasses the coiled trace 404ₐ when the layups 402ₐ, 402_{b} are properly aligned.

In reference to FIG. 9B, a proper alignment of the first and second layups 402ₐ, 402_{b}—and more specifically, the coiled trace 404ₐ and conductive layer 418—is illustrated. After alignment, any corresponding electrical features on the first and second layups 402ₐ, 402_{b} can be electrically coupled. For example, an electronic component (or another fluid-fillable or non-fluid-fillable circuit containing an electronic component) may be coupled to one or more vias and/or contact points (as shown in FIG. 8) such that it is properly integrated with the circuit 400. Notably, according to the non-limiting aspect of FIG. 9, the coiled trace 404ₐ and conductive layer 418 are configured for inductive (e.g., electromagnetic) communication and thus, no electrical connection between the first and second layups 402ₐ, 402_{b} is necessary. The alignment of FIG. 9B prepares the layups 402ₐ, 402_{b} for the bonding procedure that will result in the edge joint 416.

Accordingly, the layups 402ₐ, 402_{b} of FIG. 9B are properly aligned and prepared for the bonding process, as depicted in FIG. 9C. A seal 908 can be formed between an inner surface 413ₐ of the first layup 402ₐ and an inner surface 413_{b} of the second layup 402_{b} by any known process of attachment (e.g., welding, soldering, fusing, stitching, adhesives, etc.). The seal 408 creates an edge joint 416 between the first and second layup 402ₐ, 402_{b}. In further reference of FIG. 9C, after the edge joint 416 is formed, a substantial portion of the inner surface 413ₐ of the first layup 402ₐ can face—but is not adhered to—a substantial portion of the inner surface 413_{b} of the second layup 402_{b}. Accordingly, the seal 408, which can be formed on every side of the circuit 400, can form an internal cavity (e.g., similar to the internal cavity 110 of FIG. 2) of the fluid-fillable circuit 400. A fluid can be inserted into and contained by the internal cavity formed by the inner surfaces 413ₐ, 413_{b}, the edge joint 416, and the seal 408. As more fluid is inserted within the internal cavity, the more pressure will be exerted on the inner surfaces 413ₐ, 413_{b} of the first and second layups 402ₐ, 402_{b}. Due to the deformable nature of the layups 402ₐ, 402_{b} and traces 404ₐ, 404_{b}, the circuit 400 can be inflated, as illustrated in FIG. 9D.

It shall be appreciated that while the foregoing embodiment has been described for a structure comprising two layups, any of the foregoing structures comprising a single layup and the associated description and methods of manufacture may be similarly applied to form a system similar to the system of FIGS. 8, 9A-D.

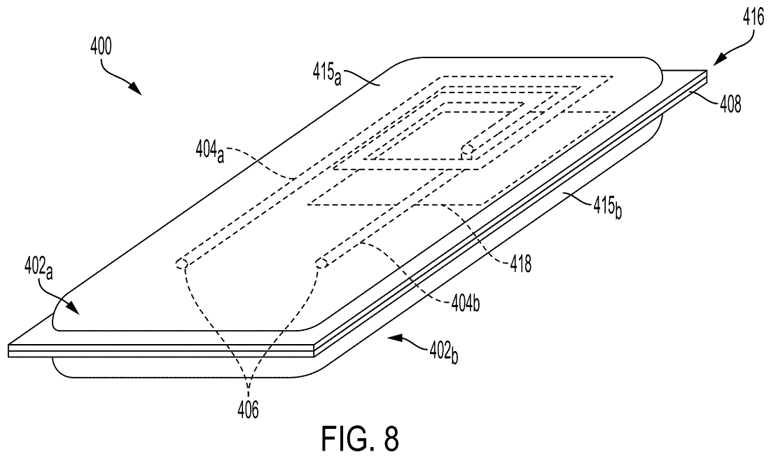

Referring now to FIG. 10, an assembly of another inflatable circuit 500 featuring an auxiliary device 520 is depicted in accordance with at least one non-limiting aspect of the present disclosure. According to the non-limiting aspect of FIG. 10, the fluid-fillable circuit 500 can include a plurality of traces 504ₐ , 504_{b} and vias 506, at least some of which are arranged such that they can be electrically coupled to an auxiliary device 520, such as an electronic component (e.g., a microprocessor, a logic-based controller, a battery, etc.) coupled to the fluid-fillable circuit 500. As such, the non-limiting aspect of FIG. 10 illustrates how a fluid-fillable circuit 500 can accommodate one or more auxiliary devices 520, which can supplement and/or enhance the electrical functionality described in reference to FIG. 8.

The fluid-fillable circuit 500 of FIG. 10 can include any of the constructions described in reference to FIGS. 1-9D. For example, according to the non-limiting aspect of FIG. 10, the circuit 500 has a single layup 502 construction. However, according to other non-limiting aspects, the fluid-fillable circuit 500 can have a multi-layup construction, such as the circuit 100 of FIG. 1. Regardless, the fluid-fillable circuit 500 of FIG. 10 defines an internal cavity via one or more joints and seals (not shown), such as the cavity 110 of FIG. 2. The internal cavity defined by the circuit 500 of FIG. 10 can accommodate and contain a fluid such that the circuit 500 can be inflated and/or deflated. However, the circuit 500 of FIG. 10 can include an alternate arrangement of traces 504ₐ, 504_{b} and vias 506 such that the circuit 500 is not complete until an auxiliary device 520 is electrically coupled to one or more via 506 of the plurality.

For example, according to the non-limiting aspect of FIG. 10, the circuit 500 can include one or more vias 506 in addition to vias positioned along a joint or seal, such as previously described vias 106 (FIG. 1), 206 (FIG. 4), 306 (FIG. 6). Notably, the vias 506 of FIG. 10 break the electrical continuity of one or more traces 504ₐ, 504_{b} positioned on the layup 502. The auxiliary device 520 can include its own electrical features positioned on its own substrate 522. According to some non-limiting aspects, the substrate 522 can be composed of the same material or have mechanical properties similar to those of the layup 502. For example, the substrate 522 can have a similar elastic modulus or other elastic properties to the layup 502, which can reduce the chances of a shear mismatch between the substrate 522 and layup 502 as the circuit 500 is inflated and/or deflated, which may result in delamination depending on the attachment, bonding or coupling method selected to attach the components to one another. Furthermore, one or more traces 524 and/or one or more vias 526 can be positioned on the substrate. According to some non-limiting aspects, the traces 524 of the auxiliary device 520 can be formed from the same deformable, conductive material or have mechanical properties similar to the material of the traces 504 of the layup 502. The vias 526 of the auxiliary device 520 can also be arranged and configured for mechanical and electrical engagement with corresponding vias 506 of the fluid-fillable circuit 500.

In further reference to FIG. 10, certain features (e.g., vias 526) of the auxiliary device 520 can be aligned with corresponding features (e.g., vias 506) of the fluid-fillable circuit 500. Once properly aligned, the auxiliary device 520 can be mechanically coupled to the layup 502 via any known process of attachment (e.g., welding, soldering, fusing, stitching, adhesives, etc.) and the vias 526 of the auxiliary device 520 can be electrically coupled to one or more of vias 506 of the layup 502. Accordingly, the traces 524 and vias 526 of the auxiliary device 520 can place a first side of a trace 504ₐ in electrical communication with a second side of the same trace 504_{b}. In other words, when the auxiliary device 520 is properly installed onto the fluid-fillable circuit 500 of FIG. 10, it completes the otherwise open circuit 500.

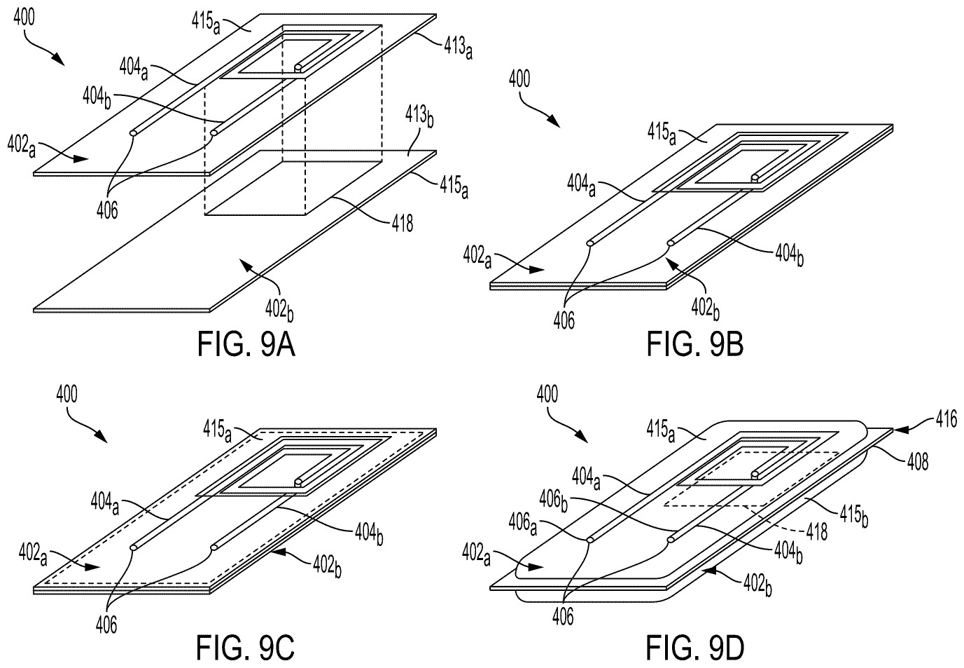

Referring now to FIG. 11, FIG a perspective view of the assembled inflatable circuit 500 of FIG. 10 is depicted in accordance with at least one non-limiting aspect of the present disclosure. According to the non-limiting aspect of FIG. 11, the auxiliary device 520 can include one or more electronic components 530 (e.g., microprocessors, logic-based controllers, memories, transmitters, receivers, transceivers, sensors, power sources, resistors, capacitors, packages, signal converters, etc.) that are mechanically coupled to the substrate 522 and electrically coupled to the traces 526. It shall be appreciated that the electronic components 530 can be particularly selected and configured in accordance with an intended application of the fluid-fillable circuit 500. For example, according to some non-limiting aspects, the one or more electronic components 530 can include a microprocessor configured to receive a signal associated with an electrical parameter from one or more traces 504ₐ, 504_{b} of the layup 502. The microprocessor can be further configured to determine a difference between detected electrical parameters and subsequently correlate the determined difference to a structural parameter (e.g., a strain, a stress, a pressure, a dimension, etc.) associated with the fluid-fillable circuit 500.

According to some non-limiting aspects, the electronic components 530 can further include a memory configured to store instructions for the microprocessor and/or a lookup table. As such, the microprocessor can correlate the determined difference in electrical parameters to a structural parameter (e.g., a strain, a stress, a pressure, a dimension, etc.) associated with the fluid-fillable circuit 500 based, at least in part, on the lookup table stored in the memory. According to other non-limiting aspects, the one or more electronic components 530 can further include a transceiver configured to transmit signals received from the layup 502 and/or generated by the microprocessor for remote processing. The transceiver can be further configured to receive remotely-generated signals that include instructions for the microprocessor 530. According to other non-limiting aspects, the microprocessor 530 can be electrically coupled to a valve assembly in fluid communication with the internal cavity and can be configured to transmit commands to the valve assembly. As such, the valve assembly can insert and/or remove fluid to and from the internal cavity in accordance with a command received from the microprocessor 530.

According to other non-limiting aspects, the one or more electronic components 530 can include a logic-based circuit (e.g., a field-programmable gate array (“FPGA”), an application specific integrated circuit (“ASIC”), etc.) configured to perform a similar functions to the microprocessor, described above. In still other non-limiting aspects, the one or more electronic components 530 can include a power source configured to store a voltage, which can be provided to the traces 504ₐ, 504_{b} of the layup 504. According to some non-limiting aspects, the one or more electronic components 530 can include a sensor (e.g., a SAW sensor, a hall-effect sensor, an ultrasonic sensor, an optical sensor, etc.). According to other non-limiting aspects, the one or more electronic components 530 can include a System-on-a-Chip (“SoC”) that includes any of the above-referenced components and/or other components that are normally found in a standard computer system.

In further reference to FIG. 11, when the auxiliary device 520 is properly installed onto the fluid-fillable circuit 500, the auxiliary device 520—and more specifically, the one or more electronic components 530—can receive and transmit a current, voltage, and/or other signals from the traces 504ₐ, 504_{b}. Notably, the auxiliary device 520 can be adhered and/or bonded to the layup 502 either prior to inflation or after inflation, depending on use preference and/or intended application. According to other non-limiting aspects, the auxiliary device 520 can be manufactured via a lamination process such that it is integral, or unitized, to the layup 502. Regardless, when properly installed, the auxiliary device 520 can operate as a functioning component of the fluid-fillable circuit 500. Alternately and/or additionally, a fluid-fillable circuit 100 may be formed such that the circuit 100 is substantially flat in an uninflated configuration but can be molded to have varied three-dimensional surface topographies and shapes with varying degrees of complexity and contour. According to such aspects, three-dimensional forming can occur at the time of inflation, for example by a thermoforming with a mold cavity having the desired final three-dimensional shape using, for example, inflation pressure. According to other non-limiting aspects, a fluid-fillable circuit 100 or system may be similarly molded in a secondary operation post-inflation, for example by thermal compression-molding in a mold cavity having the desired final 3-dimensional shape. It shall be appreciated that such methods of formation can be employed to form any of the circuits disclosed herein.

According to some non-limiting aspects, an inductive proximity sensing configuration (e.g., the coiled trace 404ₐ, and conductive layer 418 configuration of FIG. 8) can be implemented in conjunction with a strain sensing configuration (e.g., the elongating trace 504ₐ, 504_{b} configuration of FIG. 10). For example, a sensed strain can be correlated to a static internal pressure of the bladder, which can further be correlated to a spring rate for the system in which the bladder is installed, if used in a load bearing application. The sensed inductance can be correlated to a distance between features (e.g., the coiled layup 404ₐ, conductive layer 418) of the one or more layups, which can be further correlated to the spring rate to provide static or dynamic loads on a system in which the circuit is integrated.

Alternately and/or additionally, the circuit and/or electrically coupled electronic components (e.g., electronic component 530) can be configured to sense one or more of the aforementioned physical parameters associated with the circuit. For example, an inductive sensing configuration can be utilized to sense the static distance between a coil and a conductive layer, which can be used to determine an inflation pressure, a spring rate, a load, and/or other parameter associated with the circuit and/or one or more layups. A strain sensing configuration can be implemented to achieve a similar result.

Alternately and/or additionally, the circuits disclosed herein can be formed such that they can be positioned within a particular portion of an external housing, where the sensing of such parameters would be of particular interest. In other words, the circuits disclosed herein can be strategically positioned within an external housing such that the circuits are unconstrained and thus, capable of being filled with a fluid and/or inflated and sensing physical parameters in a location of interest. For example, a circuit can be formed in a fluid-fillable configuration disclosed herein, with one or more portions bonded to one or more relatively rigid structural components. However, according to such aspects, the strain sensor can be positioned away from the rigid structural components such that the bladder can experience larger deformations, and thus, can more easily sense and correlate electrical parameters associated with those deformations (e.g., force, strain rate, accelerations, loads, etc.).

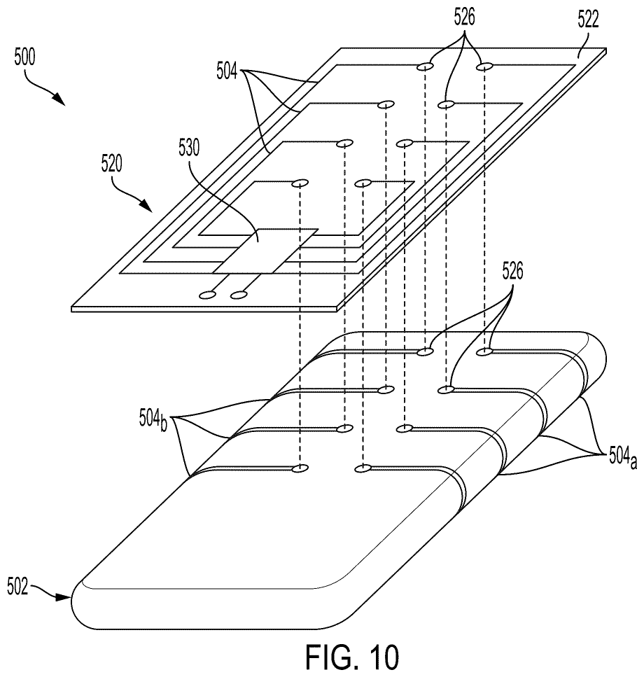

FIGS. 12A and 12B illustrate perspective views of other fluid-fillable circuits 600ₐ, 600_{b}, in accordance with at least one non-limiting aspect of the present disclosure. According to the non-limiting aspect of FIGS. 12A and 12B, each circuit 600ₐ, 600_{b} can be constructed from a single layup 602. However, according to other non-limiting aspects, either circuit 600ₐ, 600_{b} can have a multi-layup construction, such as the circuit 100 of FIG. 1. Regardless, the fluid-fillable circuit 600ₐ, 600_{b} of FIGS. 12A and 12B can define internal cavities via one or more joints and seals (not shown), such as the internal cavity 110 of FIG. 2. The internal cavity defined by the circuits 600ₐ, 600_{b} of FIGS. 12A and 12B can accommodate and contain a fluid such that the circuit 600 can be inflated and/or deflated. However, the circuit 600ₐ, 600_{b} of FIGS. 12A and 12B can include one or more light emitting diodes 608 (“LEDs”) mechanically coupled to or laminated within the layup 602 and optionally electrically coupled to one or more vias 606 by one or more traces 604. According to the non-limiting aspect of FIG. 12A, the circuit 600ₐ has LEDs 608 positioned on an upper surface of the layup 602. According to the non-limiting aspect of FIG. 12B, the circuit 600_{b} has LEDs 608 positioned on the sides of the layup 602. In both aspects, when a voltage is applied to the traces 604, the one or more LEDs 608 can be illuminated.

The one or more LEDs 608 can be implemented for cosmetic and/or functional purposes. For example, one or more of the LEDs 608 can be used in conjunction with an electronic component that includes an optical sensor, similar to the optical sensor discussed in reference to FIG. 11. As such, the optical sensor can be configured to detect photonic energy emitted by one or more of the LEDs 608, or light that was generated by one or more LEDs 608 and reflected off a surface of either inflatable circuit 600ₐ, 600_{b} and/or a surrounding structure. A microprocessor, similar to the microprocessor discussed in reference to FIG. 11, can be electrically coupled to, and configured to receive signals from, the optical sensor. The microprocessor can correlate signals received from the optical sensor to a structural parameter (e.g., a strain, a stress, a pressure, a dimension, etc.) associated with the fluid-fillable circuit 600ₐ, 600_{b}. According to another non-limiting aspect, the LEDs may be configured to illuminate in response to predetermined conditions measured, detected, calculated, inferred, correlated or sensed by a fluid-filled circuit system or assembly incorporating the principles described herein, for example with respect to FIGS. 1-11.

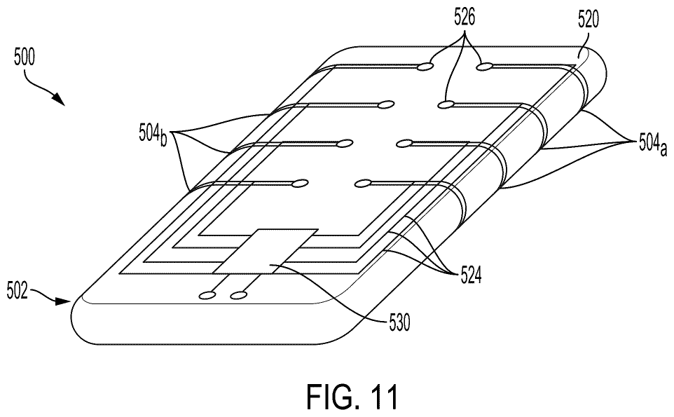

Referring now to FIG. 13, a chart 700 illustrating a correlation between an electrical parameter 702 and a structural parameter 704 of a fluid-fillable circuit is depicted in accordance with at least one non-limiting aspect of the present disclosure. According to the non-limiting aspect of FIG. 13, the fluid-fillable circuit can include an electronic component capable of sensing surface acoustic waves, such as the SAW antenna discussed in reference to FIG. 10. Accordingly, the electrical parameter 702 can include a peak amplitude of a surface acoustic wave detected by the SAW antenna. According to the non-limiting aspect of FIG. 13, the structural parameter 704 can include a dimension (e.g., length) of a trace of the fluid-fillable circuit.

According to the chart 700 of FIG. 13, as the circuit is inflated, the length of the trace increases due to the deformable composition of the length. In response to this increase in length, the electrical parameter 702, or peak amplitude of a surface acoustic wave generated by a current traversing the trace, decreases. The relationship between the electrical parameter 702 and structural parameter 704 can be mathematically characterized (e.g., via function, slope, etc.) such that a structural parameter 704 associated with the fluid-fillable circuit can be deduced based on the detected electrical parameter 702. Of course, similar models can be created for any electrical parameter in comparison to any structural parameter, in accordance with user preference and/or intended application.

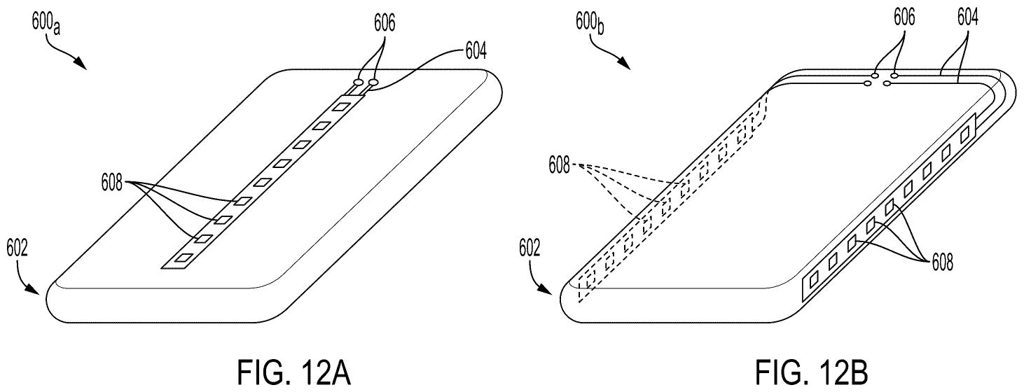

Referring now to FIG. 14, a flow chart illustrating a method 1400 of manufacturing a fluid-fillable circuit is depicted in accordance with at least one non-limiting aspect of the present disclosure. According to the non-limiting aspect of FIG. 14, the method 1400 can include correlating 1402 an electrical parameter (e.g., an inductance, a resistance, a voltage drop, a capacitance, and an electromagnetic field, etc.) of the circuit to a structural parameter (e.g., a dimension, a strain, a stress, a pressure, etc.) of the circuit. The method 1400 can further include determining 1404 an initial dimension of the deflated circuit based, at least in part, on the correlation and a final structural parameter of the inflated circuit. For example, if the circuit is to be inflated with a compressible fluid until a certain pressure is achieved within an internal cavity of the fluid-fillable circuit, a user may want to determine a final cross-sectional area or length of a trace, knowing that it will result in an electrical resistance or inductance that can be correlated with that particular pressure. The initial dimension of the trace, for example, can then be determined based on the final dimension. Once the initial dimension is determined, the method 1400 can include laminating 1406 at least a substrate layer, a deformable conductor, and an encapsulation layer, to create a layup that includes the initial dimension. Having created the layup, the method 1400 can further include sealing 1408 the layup such that an inner surface of the layup and the seal define an internal cavity, such as the internal cavity 110 of FIG. 2. Finally, the method 1400 can include inflating 1410 the circuit by filling the internal cavity with a compressible gas until a measured electrical parameter of the circuit correlates to the final structural parameter of the inflated circuit. In other words, the circuit can be inflated until a measured electrical parameter, such as resistance or inductance, correlates with the final structural parameter, such as the cross-sectional area or length. As such, a user can utilize the method 1400 of FIG. 4 to monitor electrical parameters across the circuit, to ensure the circuit is properly inflated to the correct pressure. This can be useful for benchmarking, calibration, and/or quality control, for example.

However, the steps illustrated in FIG. 14 are not the exclusive steps of the method 1400 contemplated by the present disclosure. For example, according to some non-limiting aspects, the method 1400 can further include depositing the deformable conductor within a patterned aperture of a stencil layer, thereby creating a trace of the fluid-fillable circuit assembly. According to such aspects, laminating the substrate layer, a deformable conductor, and an encapsulation layer, to create the layup can further include laminating the stencil layer. In other non-limiting aspects, the method 1400 can include laminating a second substrate layer, a second deformable conductor, and a second encapsulation layer, to create a second layup including the initial dimension, and sealing the layup to the second layup such that an inner surface of the layup, an inner surface of the second layup, and the seal define an internal cavity. Accordingly, the method 1400 can be employed to manufacture a multi-layup circuit, such as the circuit 100 of FIG. 1.

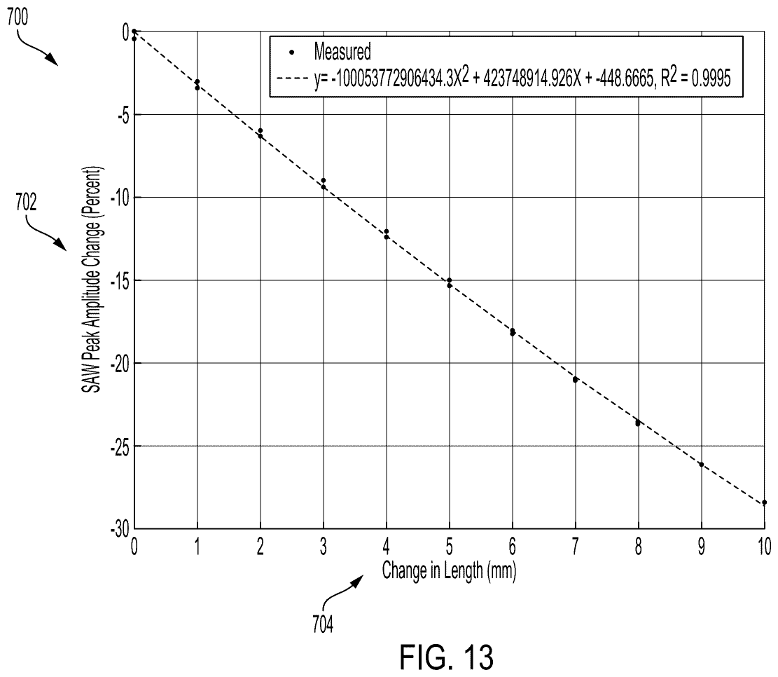

Referring now to FIG. 15, a flow chart illustrating a method 1500 of using a fluid-fillable circuit is depicted in accordance with at least one non-limiting aspect of the present disclosure. According to the non-limiting aspect of FIG. 15, the method 1500 can include applying 1502 a voltage to a trace of a fluid-fillable circuit in a first state, thereby causing a current to traverse the trace of the fluid-fillable circuit. Once a current is flowing through the circuit, the method 1500 can include detecting 1404 a first electrical parameter (e.g., an inductance, a resistance, a voltage drop, a capacitance, and an electromagnetic field, etc.) associated with the current traversing the trace of the fluid-fillable circuit in the first state. The method 1500 can further include causing 1506 the fluid-fillable circuit to transition from the first state to a second state. For example, causing 1506 the transition can include inserting a compressible fluid into an internal cavity defined by a layup of the fluid-fillable circuit. Alternately, causing 1506 the transition can include removing a compressible fluid from an internal cavity defined by a layup of the fluid-fillable circuit. According to other non-limiting aspects, causing 1506 the transition can include subjecting the fluid-fillable circuit to an external stimulus. The external stimulus can be physical (e.g., a load, an acceleration, a force, etc.), thermal (e.g., a temperature increase and/or decrease, etc.), and/or electromagnetic (e.g., an electrical load, a radio frequency, etc.) Upon inflation, the method 1500 can include detecting 108 a second electrical parameter associated with the current traversing the trace of the fluid-fillable circuit and determining 1510 a difference between the first and second electrical parameters associated with the current traversing the trace of the fluid-fillable circuit. Finally, the method can include correlating 1512 the difference between the first and second electrical parameters to a structural parameter (e.g., a strain, a stress, a pressure, a dimension, etc.) associated with the fluid-fillable circuit. Once again, the steps illustrated in FIG. 15 are not the exclusive steps of the method 1500 contemplated by the present disclosure.

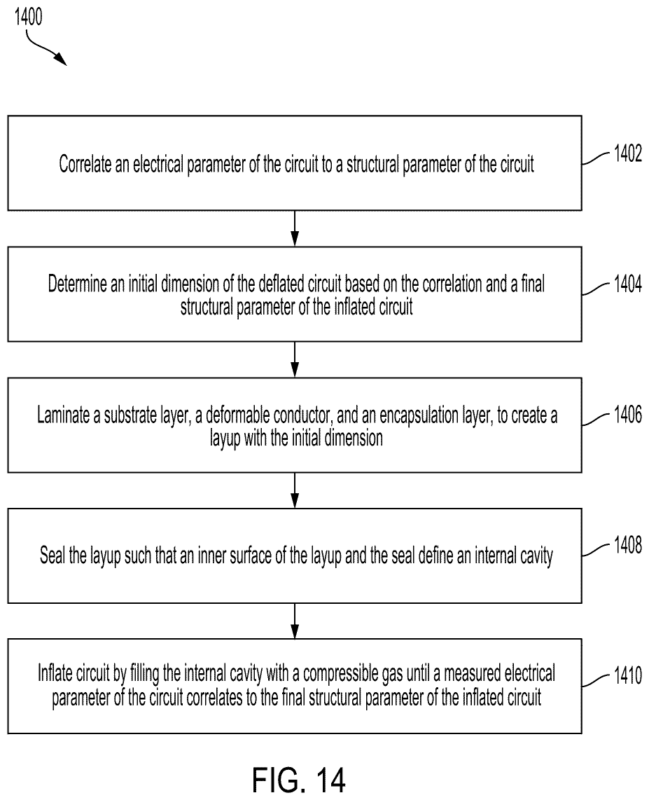

Referring now to FIG. 16, a flow chart illustrating another method 1600 of using a fluid-fillable circuit is depicted in accordance with at least one non-limiting aspect of the present disclosure. According to the non-limiting aspect of FIG. 16, the method 1600 can include identifying 1602 a target electrical parameter that correlates to a physical parameter of a circuit based on a construction and/or geometry of the circuit. For example, a target electrical parameter (e.g., resistance, inductance, etc.) can be identified for a given physical parameter (e.g., static inflation pressure, etc.) associated with the circuit based on its layup schedule and/or geometry. Next, based on this target, the method 1600 can include providing 1604 an initial electrical parameter to the circuit in a first state, such as when the circuit is not filled with a fluid. Finally, the method 1600 can include correlating 1606 the target electrical parameter and the initial electrical parameter to a circuit parameter (e.g., trace and/or trace pattern length, width, height, cross-sectional area, plan area, etc.). Once again, the steps illustrated in FIG. 15 are not the exclusive steps of the method 1500 contemplated by the present disclosure.

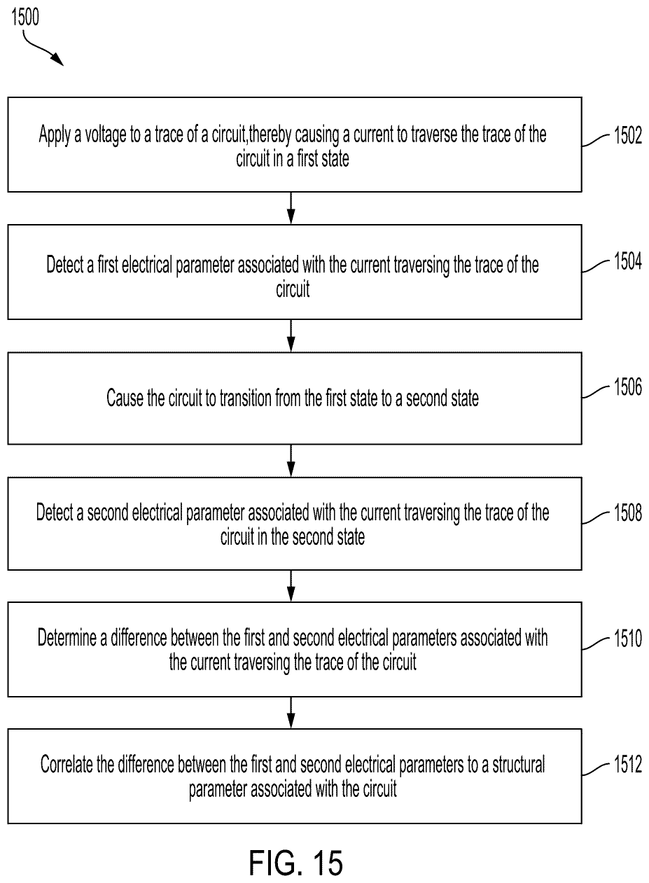

Referring now to FIG. 17, a partially fluid-fillable circuit 1700 is depicted in accordance with at least one non-limiting aspect of the present disclosure. The partially fluid-fillable circuit 1700 of FIG. 17 can include a layup 1702 comprising a first portion 1702ₐ and a second portion 1702_{b} and can be similarly constructed to the layups of the circuits discussed in reference to FIGS. 1, 4, 6, 8, 10, and 12. For example, the layup 1702 of FIG. 17 can include a multi-layer construction similar to those described in U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. Alternately and/or additionally, the layup 1702 of FIG. 17 can include a two-layer construction. The layup 1702 of FIG. 17 can be constructed from flexible structures, such as those described in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety, can be used to fabricate one or more layers of the layup 1702. For example, the layup 1702 can be produced from one or more layers including a B-stage resin film, a C-stage resin film, an adhesive, a TPU, and/or a silicone material, amongst others.

However, unlike the circuits of FIGS. 1, 4, 6, 8, 10, and 12, the first portion of the layup 1702ₐ, and the second portion of the layup 1702_{b} of FIG. 17 are arranged and mechanically coupled to one another such that the partially fluid-fillable circuit 1700 defines a fluid-fillable portion 1710ₐ and a non-fluid-fillable portion 1710_{b}. According to the non-limiting aspect of FIG. 17, a perimeter of the first portion of the layup 1702ₐ can be mechanically coupled, fused, and/or otherwise integrated to a perimeter of the second portion of the layup 1702_{b} at a mechanical interface, creating a flanged seal 1708.

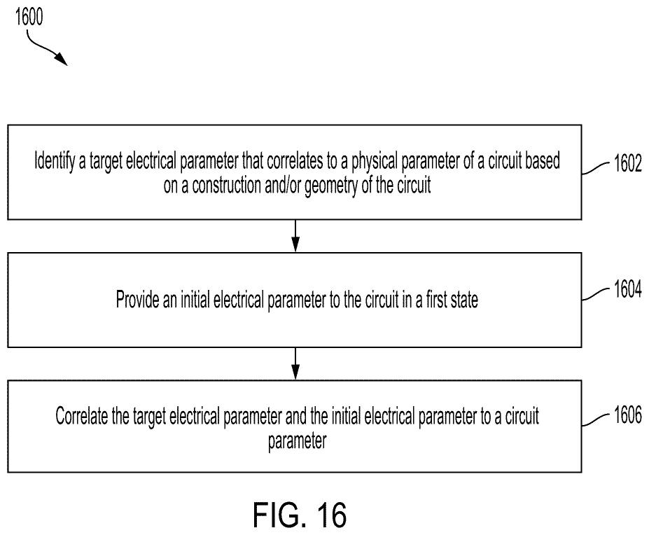

In further reference to FIG. 17, the flanged seal 1708 can be configured to define a fluid-fillable cavity of the fluid-fillable portion 1710ₐ of the partially fluid-fillable circuit 1700. The cavity can be filled with a fluid and thus, enables the fluid-fillable portion 1710, to be inflated, as previously described in reference to the circuits of FIGS. 1, 4, 6, 8, 10, and 12. However, as will be described in further detail related to FIG. 18, only the first portion of the layup 1702ₐ extends beyond the seal 1708 external the cavity and thus, the non-fluid-fillable portion 1710_{b} of the circuit 1700 cannot be filled with a fluid. Accordingly, the circuit 1700 of FIG. 17 includes a fluid-fillable portion 1710ₐ and a non-fluid-fillable portion 1710_{b} and is therefore referred to as “partially fluid-fillable.”

Although the seal 1708 of FIG. 17 traverses a substantially rectangular perimeter of the fluid-fillable portion 1710ₐ of FIG. 17 and thus, defines a cavity of substantially rectangular shape, the flanged seal 1708 and the first portion of the layup 1702ₐ can be alternately configured such that the cavity defines any desired volume and/or geometry. Accordingly, it shall be appreciated that, according to other non-limiting aspects, the fluid-fillable portion 1710ₐ can define a cavity of any number of volumes and/or shapes, according to user preference and/or intended application.

Still referring to FIG. 17, the first portion of the layup 1702ₐ and/or the second portion of the layup 1702_{b} can include one or more traces 1704 that traverse the fluid-fillable portion 1710ₐ and/or the non-fluid-fillable portion 1710_{b} of the partially fluid-fillable circuit 1700. The one or more traces 1704 of the partially fluid-fillable circuit 1700 can be formed from a deformable conductor and thus, shall include but shall not be limited to, any conductor that is in a fluid state, or any conductor that can transition into a fluid state when an external stimulus (e.g. a strain, etc.) is applied to either the conductor or a structure surrounding the conductor, wherein the conductor is also capable of returning to a pre-stimulus state. According to the non-limiting aspect of FIGS. 17 and 18, the traces 1704 of the partially fluid-fillable circuit 1700 can be configured as a simple strain sensor. However, according to other non-limiting aspects, the traces 1704 of the partially fluid-fillable circuit 1700 can be alternately and/or additionally configured as a pressure and/or proximity sensor, a bus circuit for transporting electrical power to various components, and/or an antenna circuit for transmissions, amongst other circuit types.

Alternatively and/or additionally, the one or more traces 1704 can flow within channels defined within layers of the layup 1702 construction and therefore, undergo a fluid-type strain and/or shear within the trace 1704. The traces 1704 can be formed from magnesium or a magnesium-based compound, for example, which can result in a shear thinning material that flows readily when it undergoes a shearing-type stress. Thus, when a substrate layer of the layup 1702 is relaxed and returns to pre-strained state, a magnesium trace 1704 will return to a static, or sedimentary, viscosity. In other words, the one or more traces 1704 shall flow as substrate layers of the layup 1702 are deformed and thus, will not preclude the partially fluid-fillable circuits disclosed herein from undergoing the requisite transition between a first (e.g., deflated) state to a second (e.g., inflated) state while preserving electrical communication throughout the circuit in either the first or second state. In this regard, the one or more traces 1704 can include a deformable conductor that is distinguished from conventional conductors, such as copper wires, which would prevent the transition between the first and second states, or whose rigidity might break the electrical communication throughout the circuit during said transitions. According to other non-limiting aspects, one or more layers (e.g., a substrate layer) of the layup 1702 construction can include viscoelastic properties such that the layup 1702 can absorb vibrations. Such layers of the layup can be formed from a viscoelastic film, similar to those used in a 3M™ VHB™ viscoelastic tape, for example.

For example, according to some non-limiting aspects, the one or more traces 1704 of the circuit 1700 of FIG. 17 can include a deformable, conductive material, such as those disclosed in International Patent Application No. PCT/US2017/019762 titled LIQUID WIRE, which was filed on Feb. 27, 2017 and published on Sep. 8, 2017 as International Patent Publication No. WO2017/151523A1, the disclosure of which is hereby incorporated by reference in its entirety. The one or more traces 1704 can include a variety of forms, such as a liquid, a paste, a gel, and/or a powder, amongst others that would enable the one or more traces 1704 to have a deformable (e.g., soft, flexible, stretchable, bendable, clastic, flowable viscoelastic, Newtonian, non-Newtonian, etc.) quality. According to some non-limiting aspects, the deformable, conductive materials can include an electroactive material, such as a deformable conductors produced from a conductive gel (e.g., a gallium magnesium alloy, a gallium indium alloy, and/or other gallium-based alloys, etc.). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring one or more traces 1704 of varying forms and/or compositions to achieve the benefits disclosed herein.

According to the non-limiting aspect of FIG. 17, the first portion of the layup 1702ₐ can be positioned relative to the second portion of the layup 1702_{b} such that certain features of the first portion of the layup 1702ₐ align with corresponding features of the second portion of the layup 1702_{b}. For example, similar to the circuit 100 of FIG. 1, the one or more traces 1704 of the first portion of the layup 1702ₐ can terminate in one or more electrical vias 1706, which can be aligned with one or more electrical vias (not shown) that terminate one or more traces (not shown) of the second portion of the layup 1702_{b}. Once again, according to some non-limiting aspects, it may be preferable to fill the vias 1706 with a deformable conductor configured to convey electrical energy between corresponding traces 1704. The deformable conductor placed in the vias 1706 can be the same as, similar to, or different than the deformable conductors used for the traces 1704. However, according to other non-limiting aspects, the traces 1704 can be terminated in other forms of electrical contacts and/or interconnects, in accordance with user preference and/or intended application.

Referring now to FIG. 18, an assembly of the partially fluid-fillable circuit 1700 of FIG. 17 is depicted in accordance with at least one non-limiting aspect of the present disclosure. Specifically, FIG. 18 depicts a top view of the layup 1702 of the partially fluid-fillable circuit 1700 of FIG. 17 laid flat prior to assembly. As depicted in FIG. 18, the first portion of the layup 1702ₐ and the second portion of the layup 1702_{b} are positioned about by a fold 1712 on which the layup 1702 can be folded. After having folded the layup 1702 about the fold 1712, the first portion of the layup 1702ₐ and the second portion of the layup 1702_{b} are geometrically aligned and can be sealed together about the flanged seal 1708. The seal 1708 can be formed via a process configured to attach the outer perimeters of the layup 1702, including any known process that uses heat, pressure, radio-frequency energy, and/or additional materials for attachment (e.g., welding, soldering, fusing, stitching, adhesives, etc.). Accordingly, the folded fold 1712 and the flanged seal 1708 collectively define the cavity of the fluid-fillable portion 1710ₐ of the partially fluid-fillable circuit 1700. As depicted in FIG. 18, once the first portion of the layup 1702ₐ and the second portion of the layup 1702_{b} are sealed, a part of the first portion of the layup 1702ₐ with the traces 1704 extends beyond the seal 1708, thereby forming the non-fluid fillable portion 1710ₐ of the partially fluid-fillable circuit 1700. Alternatively, the non-fluid-fillable portion 1710_{b} can be formed from a second layup attached to the fluid-fillable portion 1710ₐ, and vias may be used to establish electrical communication between traces of the non-fluid-fillable portion 1710_{b} and the fluid-fillable portion 1710ₐ. For example, vias can be positioned at flange 1708 or somewhere in the fluid-fillable (e.g., non-unitized) region.

Once assembled, the fluid-fillable portion 1710. of the circuit 1700 can be inflated using various known methods of inflation, such as the method described in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety. For example, the layup 1702 can include at least a portion of the layup 1702 can be fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 1700 of FIG. 17, and fluid can be selectively introduced and/or removed from the internal cavity via the valve assembly.

It shall be appreciated that the assembly illustrated by FIG. 18 is only one way to assemble the partially fluid-fillable circuit 1700 of FIG. 17. According to other non-limiting aspects, the first portion of the layup 1702ₐ and the second portion of the layup 1702_{b} can include substantially similar and/or overlapping geometries in a region intended to form the non-fluid-fillable portion 1710_{b}. For example, the layup can be symmetrically configured about the fold line 1712 such that the first portion of the layup 1702ₐ has the same geometry as the second portion of the layup 1702; and, when unitized about fold 1712, the first portion of the layup 1702ₐ and the second portion of the layup 1702_{b} can be unitized to form the non-fluid-fillable portion 1710_{b} of the partially fluid-fillable circuit 1700. As used herein, the term “unitize” shall include any means of mechanically coupling corresponding surfaces of any two layups, such that the unitized surfaces of those layups are mechanically inseparable in a particular region of a partially fluid-fillable circuit. In other words, surfaces of layups that are unitized cannot separate and thus, cannot define a cavity in the non-fluid-fillable portion of the partially fluid-fillable circuit. Unitization, for example, can be accomplished via the application of pressure, adhesives, and/or heat, such that two layups are mechanically inseparable in a particular region of a partially fluid-fillable circuit. However, the present disclosure contemplates other methods of unitizing two layups. Accordingly, when assembling the partially fluid-fillable circuit 1700 of FIGS. 17, an inner surface of the first portion of the layup 1702ₐ can be unitized to an inner surface of the second portion of the layup 1702_{b} at the non-fluid-fillable portion 1710_{b} of the partially fluid-fillable circuit 1700, such that the first portion of the layup 1702ₐ is integral with the second portion of the layup 1702_{b} at the non-fluid-fillable portion 1710_{b} of the partially fluid-fillable circuit 1700. However, according to this non-limiting aspect, the first portion of the layup 1702ₐ remains mechanically separate from the second portion of the layup 1702_{b} at the fluid-fillable portion 1710_{b}, such that the flanged seal 1708, fold 1712, and a boundary of the unitized portion of the first portion of the layup 1702ₐ and the second portion of the layup 1702_{b} define a fluid-fillable cavity of the fluid-fillable portion 1710ₐ of the partially fluid-fillable circuit 1700. Additionally and/or alternately, the non-fluid-fillable portion 1710_{b} can also include two or more stacks overlaid and unitized to one another.

A partially fluid-fillable circuit, such as the partially fluid-fillable circuit 1700 of FIGS. 17 and 18, can be beneficial because the fluid-fillable portion 1710ₐ can be positioned and/or integrated into a housing at a desired location for particular functions (e.g., sensing at a particular location of interest, relative movement of an electronic component positioned at a particular location of interest, etc.), while maintaining electrical continuity with the non-fluid-fillable portion 1710_{b}. The non-fluid-fillable portion 1710_{b} can be positioned and/or integrated into a housing at another desired location, for example a location of the housing where the benefits of an inflated or selectively inflatable bladder are not required, but where it may be desirable to route and conduct signals or electrical currents, and/or provide other types of sensing structures formed from traces made from the deformable conductor. This can be beneficial for integrating the partially fluid-fillable circuit 1700 into wearable articles and promoting comfort without compromising the utility of the partially fluid-fillable circuit 1700.

Additionally and/or alternately, it may be desirable to couple certain electronic components to the fluid-fillable portion 1710ₐ and/or the the non-fluid-fillable portion 1710_{b}. For example, according to the non-limiting aspect of FIGS. 17 and 18, the traces 1704 can be configured as a simple sensor, such that the traces 1704 generate varying electrical parameters (e.g., an inductance, a resistance, a voltage drop, a capacitance, signal, an electromagnetic field, etc.) as an electrical current and/or potential is be applied to the circuit 1700 and the fluid-fillable portion 1710ₐ is inflated and/or deflated or the non-fluid-fillable portion 1710_{b} is stretched and/or otherwise deformed. In some examples, it might be beneficial to couple one or more electronic components (e.g., a power source, a microprocessor, a logic-based controller, a transceiver, an electrode, a LED bank, a pump etc.) which may be mechanically secured to and/or otherwise integrated into the first portion of the layup 1702ₐ at the non-fluid-fillable portion 1710_{b} of the partially fluid-fillable circuit 1700.

For example, a microprocessor, on its own or hosted as part of an auxiliary component such as on a flex printed circuit board (“PCB”), can be coupled to the non-fluid-fillable portion 1710_{b} and configured to correlate the varying electrical parameters (e.g., an inductance, a resistance, a voltage drop, a capacitance, signal, an electromagnetic field, etc.) to a structural parameter (e.g., a strain, a stress, a pressure, a dimension, etc.). Alternately and/or additionally, either a receiver, a transmitter, or a transceiver can be coupled to the non-fluid-fillable portion 1710_{b} and configured to transmit and/or receive signals to and/or from the partially fluid-fillable circuit 1700 and/or its various electronic components. Alternately and/or additionally, an LED array can be coupled to the non-fluid-fillable portion 1710_{b} and configured to illuminate one or more LEDs based on electrical parameters (e.g., an inductance, a resistance, a voltage drop, a capacitance, signal, an electromagnetic field, etc.) generated by the traces 1704 and/or structural parameters (e.g., a strain, a stress, a pressure, a dimension, etc.) determined by a microprocessor. Accordingly, various electronic components can be coupled to non-fluid-fillable portion 1710_{b} and configured to monitor, transmit, and/or otherwise dynamically or statically calculate, infer or determine one or more physical or structural characteristics or conditions of the partially fluid-fillable circuit 1700, and/or a stimuli applied to the partially fluid-fillable circuit 1700.

It shall be appreciated that, according to non-limiting aspects wherein the partially fluid-fillable circuit 1700 is integrated into a wearable article, comfort can be promoted by mounting various electronic components to the non-fluid-fillable portion 1710_{b}, as the electronic components will not be pressed against the user's body, joint, or appendage as the fluid-fillable portion 1710ₐ is selectively inflated and deflated. However, according to other non-limiting aspects, it may be beneficial to coupled certain electronic components (e.g., electrodes) to the fluid-fillable portion 1710ₐ of the partially fluid-fillable circuit 1700. For example, according to some non-limiting aspects, wherein the partially fluid-fillable circuit 1700 is integrated into a wearable article, an electrode can be coupled to the fluid-fillable portion 1710ₐ of the partially fluid-fillable circuit 1700 and configured to monitor and/or stimulate a user's body part at a particular location. Accordingly, as the fluid-fillable portion 1710_{b} of the partially fluid-fillable circuit 1700 is inflated and/or deflated, the electrode can be biased against the user, according to user preference and/or intended application.

In some non-limiting aspects, the microprocessor can be used to monitor the signal generated by an electrode (e.g., an EMG) to assist in determining the correct amount of biasing required from the inflatable portion. A feedback system can be provided (e.g., a system with a processor or microprocessor) electrically coupled to one or more electrodes, and a inflation pump electrically coupled with the processor, which may configured to inflate or deflate the fluid fillable portion 1710ₐ of the partially-fluid-fillable circuit 1700 to optimize the signal received from the electrode, in response to commands received from the processor. Sensors integrated to the fluid-fillable portion 1710ₐ may transmit signals to the processor, which may actively monitor the pressure within the partially-fluid-fillable portion 1710ₐ. As such, the feedback system can prevent the fluid-fillable portion 1710ₐ from becoming filled beyond a predetermined threshold (e.g., over-inflation, under-inflation, etc.) or can set an inflation range or threshold value specified by a user that corresponds to an acceptable level of comfort.

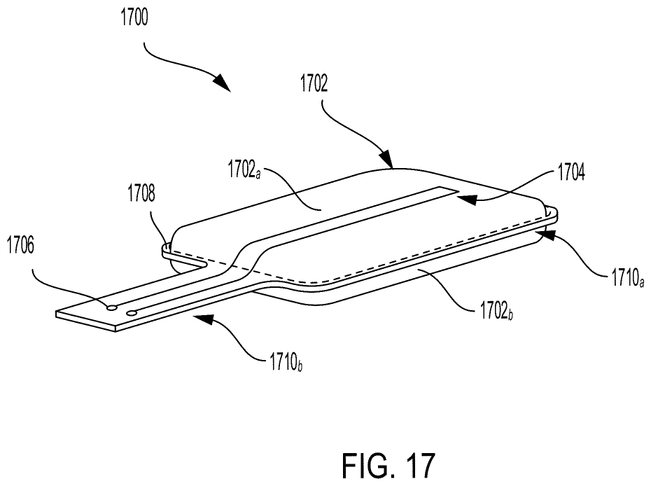

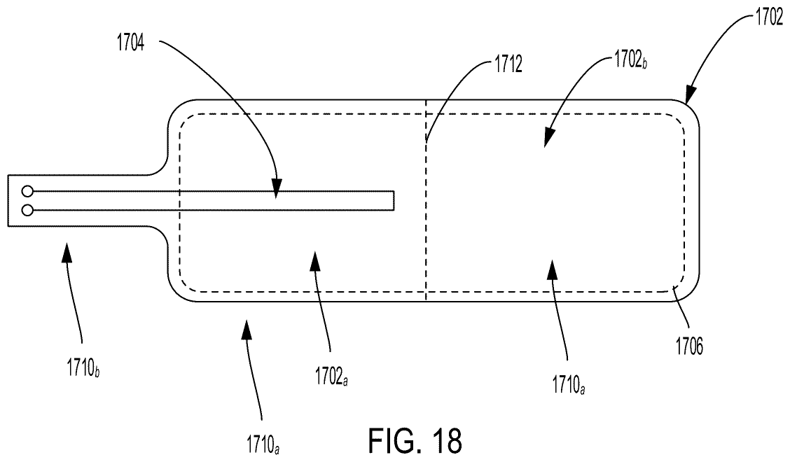

Referring now to FIG. 19, another partially fluid-fillable circuit 1900 is depicted in accordance with at least one non-limiting aspect of the present disclosure. According to the non-limiting aspect of FIG. 19, the partially fluid-fillable circuit 1900 can include at least a first layup 1902ₐ mechanically coupled to a second layup 1902_{b}, e.g., at a seal 1908, as will be discussed in further detail in reference to FIG. 20. The first layup 1902ₐ can include one or more traces 1904 formed from a deformable conductor, which traverse the fluid-fillable portion 1910ₐ, the first non-fluid-fillable portion 1910_{b}, and the second non-fluid-fillable portion 1910ₐ of the partially fluid-fillable circuit 1900. Alternatively, one of the the fluid-fillable portion 1910ₐ and the non-fluid-fillable portion 1910_{b} may be provided on one of the first layup 1902ₐ and the the second layup 1902_{b}, and the other of the fluid-fillable portion 1910ₐ and the non-fluid-fillable portion 1910_{b} may be provided on the other of the first layup 1902ₐ and the second layup 1902_{b}. The one or more traces 1904 can terminate in one or more electrical vias 1906—or other electrical interconnects—positioned on the first non-fluid-fillable portion 1910_{b}, and the second non-fluid-fillable portion 1910_{c} of the partially fluid-fillable circuit 1900. The first layup 1902ₐ, and second layup 1902_{b} of FIG. 19 can be configured similar to the layup 1702 of FIG. 17. Likewise, the deformable conductor of the traces 1904 and electrical vias 1906 of FIG. 19 can be configured similar to the deformable conductor of the traces 1704 and electrical vias 1706 of FIG. 17, respectively. It shall be further appreciated that either and/or both of the non-inflatable portions can be mechanically and electrically coupled to the inflatable portion and not formed integrally with either of the first and second layups that form the inflatable portion as well

In further reference to FIG. 19, the flanged seal 1908 is configured to define a fluid-fillable cavity of a fluid-fillable portion 1910ₐ of the partially fluid-fillable circuit 1900. The cavity can be filled with a fluid and thus, enables the fluid-fillable portion 1910ₐ to be inflated, similar to the partially fillable circuit 1700 of FIG. 17 and as previously described in reference to the circuits of FIGS. 1, 4, 6, 8, 10, and 12. However, as will be described in further detail related to FIG. 20, a first and second portion 1912_{b}, 1912_{c} of the first layup 1902ₐ traverses beyond the seal 1908 external to the cavity and thus, the non-fluid-fillable portions 19106, 1910, of the circuit 1900 cannot be filled with a fluid. Accordingly, the circuit 1900 of FIG. 19 includes a fluid-fillable portion 1910ₐ and two non-fluid-fillable portions 1910_{b}, 1910_{c} and thus, is partially fluid-fillable.

Although the seal 1908 of FIG. 19 traverses a substantially rectangular perimeter of the fluid-fillable portion 1910ₐ of FIG. 19, thereby defining a cavity of substantially rectangular shape, the flanged seal 1908 and the first and second layups 1902ₐ, 1902_{b} can be alternately configured such that the cavity defines any desired volume and/or geometry. Accordingly, it shall be appreciated that, according to other non-limiting aspects, the fluid-fillable portion 1910ₐ can define a cavity of any number of volumes and/or shapes, according to user preference and/or intended application.

Referring now to FIG. 20, an assembly of the partially fluid-fillable circuit 1900 of FIG. 19 is depicted in accordance with at least one non-limiting aspect of the present disclosure. Specifically, FIG. 19 depicts a top view of the first layup 1902. and the second layup 1902_{b} of the partially fluid-fillable circuit 1900 of FIG. 19 laid flat prior to assembly. According to the non-limiting aspect of FIG. 20, the first layup 1902ₐ can include a first portion 1912ₐ, a second portion 1912_{b}, and a third portion 1912_{c}, each of which can be configured to correspond to the fluid-fillable portion 1910ₐ or the non-fluid-fillable portion 1910_{b} of the partially fluid-fillable circuit 1900, as depicted in FIG. 19. According to the non-limiting aspect of FIG. 20, a first portion 1912ₐ of the first layup 1902ₐ can include a substantially similar geometry that corresponds to the entire geometry of the second layup 1902_{b}. As such, the first portion 1912ₐ of the first layup 1902ₐ can be positioned above and aligned with the second layup 1902_{b}. After having positioned the first layup 1902ₐ above the second layup 1902_{b}, the second layup 1902_{b} can be sealed to the first layup 1902ₐ about the seal 1908, which traverses a perimeter of the first portion 1912ₐ of the first layup 1902ₐ.

Once again, the seal 1908 can be formed via a process configured to attach an outer perimeters of the second layup 1902_{b} to the first portion 1912ₐ of the first layup 1902ₐ, including any known process that uses heat, pressure, radio-frequency energy, and/or additional materials for attachment (e.g., welding, soldering, fusing, stitching, adhesives, etc.). Accordingly, an inner surface of the first portion 1912ₐ of the first layup 1902ₐ, and inner surface of the second layup 1902_{b}, and the seal 1908 collectively define the cavity of the fluid-fillable portion 1910ₐ of the partially fluid-fillable circuit 1900. As depicted in FIG. 19, once the first portion 1912ₐ of the first layup 1902ₐ is mechanically coupled to the second layup 1902ₐ via seal 1908, the second portion 1912, and the third portion 1912_{b} of the first layup 1902ₐ extend beyond the seal 1908, thereby forming the non-fluid fillable portions 1910_{b}, 1910_{c} of the partially fluid-fillable circuit 1900 of FIG. 19.

Upon assembly, the fluid-fillable portion 1910ₐ of the circuit 1900 of FIG. 19 can be inflated using several methods of inflation, including those discussed in reference to FIG. 18. For example, the first layup 1902ₐ and/or the second layup 1902_{b} can include at least a portion fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 1900 of FIG. 19, and fluid can be selectively introduced and/or removed from the internal cavity via the valve assembly. Accordingly, the partially fluid-fillable circuit 1900 of FIG. 19 can realize all of the benefits described in reference to the circuit 1700 of FIGS. 17 and 18, including a more precise integration into a housing, the particular placement of electronic components, and an overall enhanced comfort for a user when integrated into a wearable article.

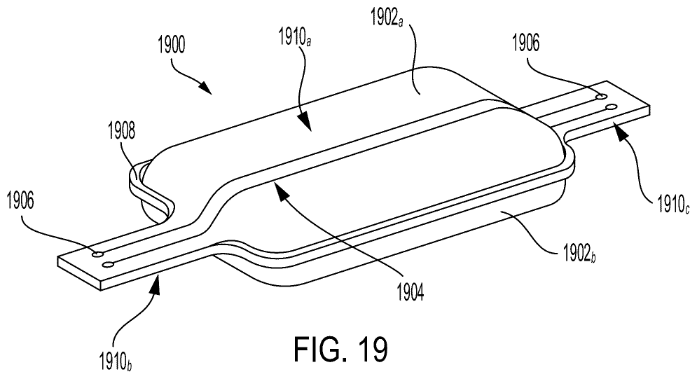

Referring now to FIG. 21, another assembly of another partially fluid-fillable circuit 2100 is depicted in accordance with at least one non-limiting aspect of the present disclosure. Specifically, FIG. 21 depicts a top view of a single layup 1902 configured with a fold 2112. According to the non-limiting aspect of FIG. 21, the partially fluid-fillable circuit 2100 can include a layup 2102 that includes a first portion 2101ₐ, a second portion 2101_{b}, a third portion 2101_{c}, and a first portion 2101_{d}. The first portion 2102ₐ and the second portion 2102_{b} of the layup 2102 have substantially similar geometries that correspond to one another and are disposed about the fold 2112. As will be described in further detail, the first and second portions 2102ₐ, 2102_{b} of the layup 2102 can be configured to define a fluid-fillable portion of the partially fluid-fillable circuit 2100. Once again, the layup 2102 can include one or more traces 2104 formed from a deformable conductor, which can traverse a fluid-fillable portion defined the first and second portions 2102ₐ, 2102_{b} of the layup 2102. The one or more traces 2104 can terminate in one or more electrical vias 2106—or other electrical interconnects—positioned on the third and fourth portions 2102_{c}, 2102_{d} of the layup 2102. The layup 2102 of FIG. 21 can be configured similar to the layup 1702 of FIG. 17. Likewise, the deformable conductor of the traces 2104 and electrical vias 2106 of FIG. 21 can be configured similar to the deformable conductor of the traces 1704 and electrical vias 1706 of FIG. 17, respectively. Alternately and/or additionally, a non-fluid-fillable portion of the partially fluid-fillable circuit 2100 can be formed from a second layup attached to the the first and second portions 2102ₐ, 2102_{b} of the layup 2102 configured to define a fluid-fillable portion fluid-fillable portion. Once again, vias can be used to establish electrical communication between traces of the non-fluid-fillable portion and the first and second portions 2102ₐ, 2102_{b} of the layup 2102 configured to define a fluid-fillable portion fluid-fillable portion. For example, vias can be positioned at flange or somewhere in the fluid-fillable (e.g., non-unitized) region.

As depicted in FIG. 21, the first portion of the layup 2102ₐ and the second portion of the layup 2102_{b} are positioned about by a fold 2112 on which the layup 2102 can be folded. After having folded the layup 2102 about the fold 2112, the first portion of the layup 2102ₐ and the second portion of the layup 2102_{b} are geometrically aligned and can be sealed together about the flanged seal 2108. Once again, the seal 2108 can be formed via a process configured to attach the outer perimeters of the first and second portions 2102ₐ, 2102_{b} of the layup 2102, including any known process that uses heat, pressure, radio-frequency energy, and/or additional materials for attachment (e.g., welding, soldering, fusing, stitching, adhesives, etc.). Accordingly, the folded fold 2112 and the flanged seal 2108 can collectively define a cavity of a fluid-fillable portion of the partially fluid-fillable circuit 2100. As depicted in FIG. 21, once the first portion of the layup 2102ₐ and the second portion of the layup 2102_{b} are sealed, the third and fourth portions 2102_{c}, 2102_{d} of the layup 2102 with the traces 2104 can extend beyond the seal 2108, thereby forming a non-fluid fillable portion of the partially fluid-fillable circuit 2100.

Although the seal 2108 and the fold 2112 of FIG. 21 collectively traverse a substantially rectangular perimeter of the first and second portions 2102ₐ, 2102_{b} of the layup 2102 of FIG. 21, thereby defining a cavity of substantially rectangular shape, the seal 2108, fold 2112, and layup 2102 can be alternately configured such that the cavity defines any desired volume and/or geometry. Accordingly, it shall be appreciated that, according to other non-limiting aspects, the seal 2108, fold 2112, and layup 2102 can be alternately configured to define a fluid-fillable portion with a cavity of any number of volumes and/or shapes, according to user preference and/or intended application.

Once assembled, the partially fluid-fillable circuit 2100 can be substantially similar to the partially fluid-fillable circuit 1900 of FIG. 19, except the folded fold 2112 along with the flanged seal 2108 define the cavity, along with an inner surface of the first portion 2102ₐ and an inner surface of the second portion 2102_{b} of the layup 2102. Upon assembly, the fluid-fillable portion of the circuit 2100 of FIG. 21 can be inflated using several methods of inflation, including those discussed in reference to FIG. 18. For example, the first portion 2102ₐ of the layup 2100 and/or the second portion 2102_{b} of the layup 2102 can include a multi-layer construction and can be fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 2100 of FIG. 21, and fluid can be selectively introduced and/or removed from the internal cavity via the valve assembly. Accordingly, the partially fluid-fillable circuit 2100 of FIG. 21 can realize all of the benefits described in reference to the circuit 1700 of FIGS. 17 and 18, including a more precise integration into a housing, the particular placement of electronic components, and an overall enhanced comfort for a user when integrated into a wearable article.

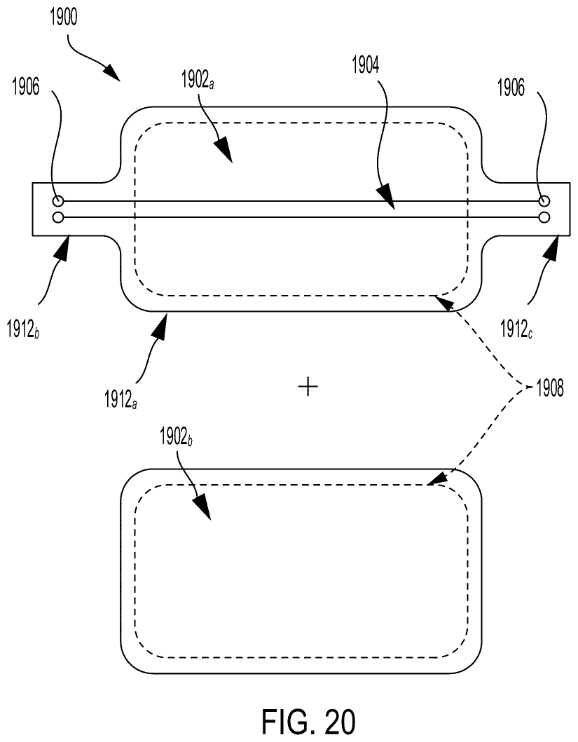

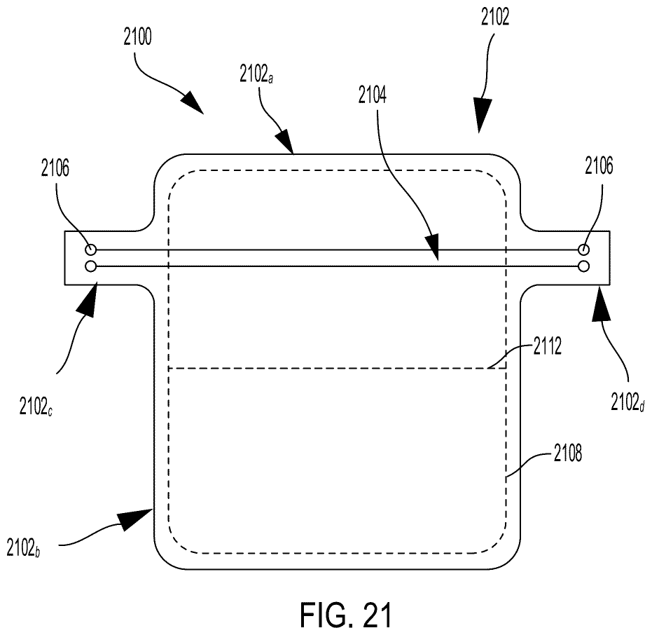

Referring now to FIG. 22, another partially fluid-fillable circuit 2200 is depicted in accordance with at least one non-limiting aspect of the present disclosure. According to the non-limiting aspect of FIG. 22, the partially fluid-fillable circuit 2200 can include a first layup 2202ₐ, mechanically coupled to a second layup 2202_{b} via a seal 2208, as previously discussed. Additionally, one or more traces 2204 formed from a deformable conductor can traverse an external surface of the first layup 2202ₐ and/or a non-fluid-fillable portion 2210_{b} of the partially fluid-fillable circuit 2200 that traverses through the cavity of the fluid-fillable portion 2210ₐ. The non-fluid-fillable portion 2210_{b} will be described in further detail in reference to FIG. 23. The first layup 2202ₐ, and second layup 2202_{b} of FIG. 22 can be configured similar to the layup 1702 of FIG. 17. Likewise, the deformable conductor of the traces 2204 of FIG. 22 can be configured similar to the deformable conductor of the traces 1704 of FIG. 17. Alternately, it shall be appreciated that, according to some non-limiting aspects, the partially fluid-fillable circuit 2200 of FIG. 22 can be formed from a single layup.

According to the non-limiting aspect of FIG. 22, the partially fluid-fillable circuit 2200 can include a contained geometry, since the non-fluid-fillable portion 2210_{b} traverses through the cavity of the fluid-fillable portion 2210ₐ and is completely encompassed by the the fluid-fillable portion 2210ₐ. Accordingly, any traces 2204 and/or electronic components (e.g., a power source, a microprocessor, a logic-based controller, a transceiver, an electrode, one or more LEDs, a pump etc.) of the non-fluid-fillable portion 2210_{b} can be completely encompassed by the fluid-fillable portion 2210ₐ of the partially fluid-fillable circuit 2200. These features can enable the partially fluid-fillable circuit 2200 to be particularly useful for a variety of end-uses including, without limitation, a midsole of a shoe, a personal massage device, protective padding, and/or a recreational ball, all of which are examples of end-uses that could benefit from the contained geometry and can be selectively inflated and/or deflated according to user preference and/or intended application.

Referring now to FIG. 23, a cross-sectioned side view of the partially fluid-fillable circuit 2200 of FIG. 22 is depicted in accordance with at least one non-limiting aspect of the present disclosure. Specifically, the cross-section of FIG. 23 is taken along line A-A, as depicted in FIG. 22. Accordingly, the first layup 2202ₐ and second layup 2202_{b} are mechanically coupled via the seal 2208, which can be formed via any known process that uses heat, pressure, radio-frequency energy, and/or additional materials for attachment (e.g., welding, soldering, fusing, stitching, adhesives, etc.), thereby defining a cavity 2215 between the inner surfaces of the first and second layups 2202ₐ, 2202_{b}. As such, the outer surfaces of the first and second layups 2202ₐ, 2202_{b} define a fluid-fillable portion 2210ₐ of the partially fluid-fillable circuit 2200.

The seal 2208 of FIGS. 22 and 23 traverses a substantially rectangular perimeter of the fluid-fillable portion 2210ₐ, thereby imbuing the cavity 2215 with a substantially rectangular shape and volume. However, the flanged seal 2208 and the first and second layups 2202ₐ, 2202_{b} can be alternately configured such that the cavity 2215 defines any desired volume and/or geometry. Accordingly, it shall be appreciated that, according to other non-limiting aspects, the fluid-fillable portion 2210ₐ can define a cavity 2215 of any number of volumes and/or shapes, according to user preference and/or intended application.

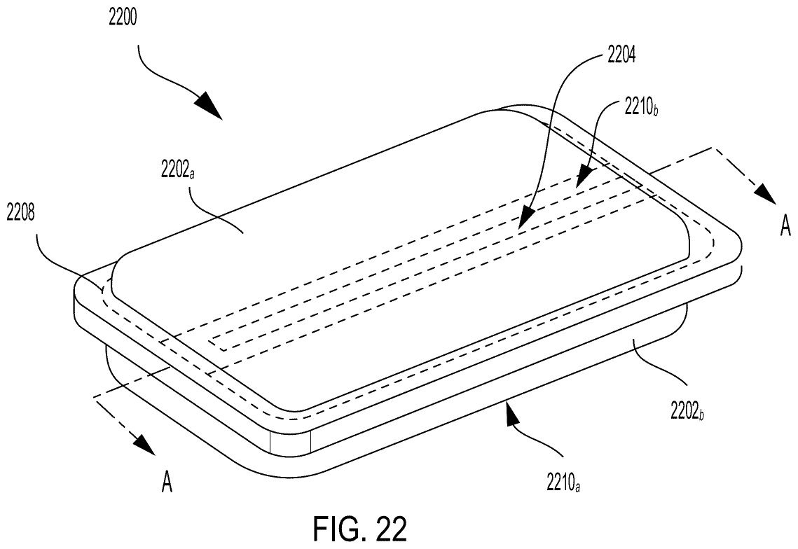

Notably, the partially fluid-fillable circuit 2200 of FIGS. 22 and 23 can include a non-fluid-fillable portion 2210_{b} that traverses through the cavity 2215 defined by and intermediate to the first and second layups 2202., 22026. As previously discussed, the one or more traces 2204 formed from a deformable can traverse the first layup 2202ₐ that, as will be described in further detail in reference to FIG. 24, can be unitized at the seal 2208 to a third layup 2202_{c} that defines the non-fluid-fillable portion 2210_{b}, which traverses the cavity through 2215. According to the non-limiting aspect of FIG. 23, the first layup 2202ₐ and the third layup 2202_{c} can include, at least, a substrate layer 2217 and an encapsulation layer 2219, as previously described. The deformable conductor that defines the traces 2204 can be deposited directly on the substrate layer 2217 and encapsulated by the encapsulation layer 2219. However, according to other non-limiting aspects, the first layup 2202ₐ can further include a stencil layer that defines a channel in which the deformable conductor can be deposited, thereby forming the traces 2204.

Upon assembly, the fluid-fillable portion 2210ₐ of the circuit 2200 can be inflated using several methods of inflation, including those discussed in reference to FIG. 18. For example, the first layup 2202ₐ and/or the second layup 2202_{b} can include at least a portion fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 2200 of FIG. 23, and fluid can be selectively introduced and/or removed from the internal cavity 2215 via the valve assembly. Accordingly, the partially fluid-fillable circuit 2200 of FIG. 23 can realize all of the benefits described in reference to the circuit 1700 of FIGS. 17 and 18, including a more precise integration into a housing, the particular placement of electronic components, and an overall enhanced comfort for a user when integrated into a wearable article.

Referring now to FIG. 24, an assembly of the partially fluid-fillable circuit 2200 of FIG. 22 is depicted in accordance with at least one non-limiting aspect of the present disclosure. According to the non-limiting aspect of FIG. 24, the particular constructions of the first layup 2202ₐ, the second layup 2202_{b}, and the third layup 2202_{c} are depicted in further detail. For example, the substrate layer 2217 and encapsulation layer 2219 are depicted as encompassing the deformable conductor that defines the traces 2204 of the partially fluid-fillable circuit 2200. According to the non-limiting aspect of FIG. 24, the traces 2204 of the first layup 2202ₐ and the third layup 2202_{c} can be electrically coupled via one or more electrical vias 2206, which can be placed within the flanged seal 2208. Additionally, according to some non-limiting aspects, the first and third layups 2202ₐ, 2202_{c} can include a stencil layer 2221 that includes channels in which the deformable conductor can be deposited, thereby providing a more robust structural formation of the traces 2204. The traces 2204 in the first layup 2202. that defines an outer wall of the cavity 2215 (FIG. 23) can be optional and, according to some non-limiting aspects, only the third layup 2202_{c} that defines the non-fluid-fillable portion 2210_{b} of the circuit 2200 can include traces 2204.

According to the non-limiting aspect of FIG. 24, the second layup 2202_{b} includes a single layer construction that does not contain any traces 2204 and thus, is simply configured to function as an outer wall of the cavity 2215 (FIG. 23). However, according to other non-limiting aspects, any of the first layup 2202ₐ, the second layup 2202_{b}, and third layup 2202_{c} can include traces 2204 formed from a deformable conductor and/or varying constructions involving various layers. Furthermore, although the traces 2204 of FIG. 24 are configured such that the circuit 2200 functions as a simple strain gauge, it shall be appreciated that, according to other non-limiting aspects, the traces 2204 of the partially fluid-fillable circuit 2200 can be alternately and/or additionally configured to function as a pressure and/or proximity sensor, a bus circuit for transporting electrical power to various components, and/or an antenna circuit for transmissions, amongst other circuit types.

In further reference to FIG. 24, bond lines 2220ₐ, 2220_{b} of the flanged seal 2208 is depicted about which the first layup 2202ₐ, the second layup 2202_{b}, the third layup 2202_{c} can be unitized. According to the non-limiting aspect of FIG. 24, the first layup 2202ₐ and third layup 2202_{c} are unitized about a first bond line 2220ₐ, thereby forming a portion of the flanged seal 2208. However, the second layup 2202_{b} has yet to be unitized to the first layup 2202ₐ and third layup 2202_{c} about the bond line 2220 of the flanged seal 2208. Once the second layup 2202_{b} is unitized to the first layup 2202ₐ and the third layup 2202_{c} about the first bond line 2220ₐ, the flanged seal 2208 will be complete and the cavity 2215 (FIG. 23) can be filled with a fluid.

Since the partially fluid-fillable circuit 2200 of FIG. 24 is assembled without folding, it can be beneficial if adjacent surfaces of the first layup 2202ₐ, the second layup 2202_{b}, and the third layup 2202_{c} are properly spaced during unitization. For example, according to non-limiting aspects where a plate heat press is being used in the unitizing process, a user should ensure that there is some separation and/or a release liner (e.g., a PTFE film) that prevents the layers from being unitized. Proper spacing can ensure proper mechanical separation and unitization in the desired locations. For example, unitization may only be desired at the flanged seal 2208. Accordingly, separation of adjacent surfaces of the first layup 2202ₐ, the second layup 2202_{b}, and the third layup 2202_{c} can be achieved via a release liner positioned between portions of the surfaces that are intended to be separated. After the layups 2202ₐ, 2202_{b}, 2202_{c} are unitized at the flanged seal 2208, the releasable liner can be removed such that adjacent surfaces of the first layup 2202ₐ, the second layup 2202_{b}, and the third layup 2202_{c} define a cavity 2215 (FIG. 23), as desired.

However, precise spacing may not be necessary. For example, according to some non-limiting aspects, adjacent surfaces of the first layup 2202ₐ, the second layup 2202_{b}, and the third layup 2202_{c} can be placed in contact and a unitizing tool can include features that only contact the areas to be unitized, reducing the need for precise spacing and increasing the manufacturing margin of error.

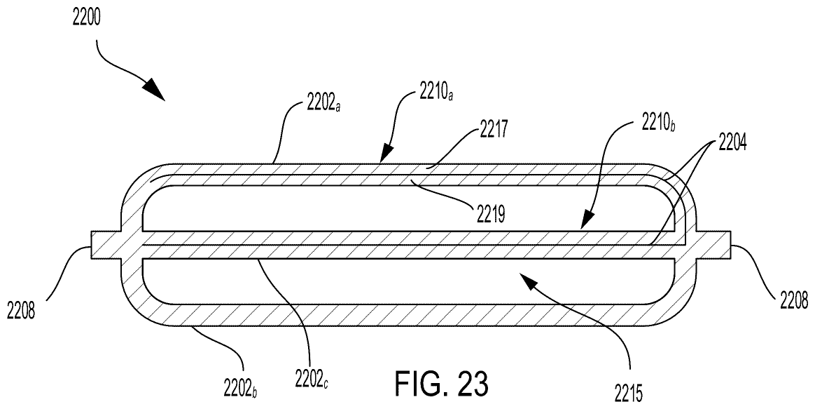

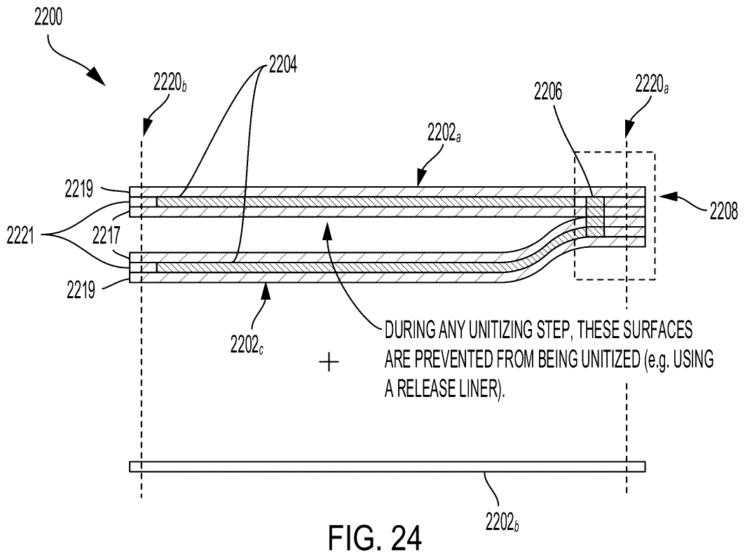

Referring now to FIG. 25, another partially fluid-fillable circuit 2500 is depicted in accordance with at least one non-limiting aspect of the present disclosure. According to the non-limiting aspect of FIG. 25, the partially fluid-fillable circuit 2500 can include a first layup 2502ₐ, mechanically coupled to a second layup 2502_{b} (FIGS. 26A-C) via a seal 2508. Similar to the circuit 2200 of FIG. 22, a non-fluid-fillable portion 2510_{b} traverses through a cavity defined by a fluid-fillable portion 2510ₐ of the partially fluid-fillable circuit 2500. However, unlike the circuit 2200 of FIG. 22, a portion of the non-fluid-fillable portion 2510_{b} is configured as a “tongue” that traverses beyond the flanged seal 2508 external to the cavity of the fluid-fillable portion 2510ₐ. As depicted in FIG. 25, one or more traces 2504 can be formed from a deformable conductor and configured to traverse the portion of the first layup 2502ₐ that forms the non-fluid-fillable portion 2510_{b} of the partially fluid-fillable circuit 2500.

As will be described in further detail in reference to FIGS. 26A-C, one or more electronic components, such as an LED array 2520, can be electrically coupled to the traces 2504 and positioned within the cavity defined by the fluid-fillable portion 2510ₐ of the partially fluid-fillable circuit 2500 of FIG. 25. It shall be appreciated that any of the previously disclosed circuits of FIGS. 17-24 can also be configured to include a one or more electronic components in a similar fashion. Furthermore, the one or more traces 2504 can terminate in one or more electrical vias 2506—or other electrical interconnects—positioned on the first non-fluid-fillable portion 2510_{b}, of the partially fluid-fillable circuit 2500. The first layup 2502ₐ, and second layup 2502_{b} of FIG. 25 can be configured similar to the layup 1702 of FIG. 17. Likewise, the deformable conductor of the traces 2504 and electrical vias 2506 of FIG. 25 can be configured similar to the deformable conductor of the traces 1704 and electrical vias 1706 of FIG. 17, respectively.

Referring now to FIGS. 26A-C, an assembly of the partially fluid-fillable circuit 2200 of FIG. 22 is depicted in accordance with at least one non-limiting aspect of the present disclosure. As depicted in the top view of FIG. 26A, the first layup 2202ₐ can include a substantially rectangular portion and an elongated portion that extends from the substantially rectangular portion. According to the non-limiting aspect of FIG. 26A, the traces 2504 and LED bank 2520 can traverse the elongated portion. In further reference to FIG. 26A, the second layup 2502_{b} can have a substantially rectangular geometry that corresponds to the substantially rectangular portion of the first layup 2502ₐ. Bond lines 1 and 2 are depicted on either end of the second layup 2502_{b}, marking portions of the second layup 2502_{b} that will be bonded to the first layup 2502ₐ in a particular sequence, as depicted in FIGS. 26B and 26C.

For example, referring to FIG. 26B, a cross-section of the partially fluid-fillable circuit 2500 taken about line A-A is depicted in accordance with at least one non-limiting aspect of the present disclosure. According to the non-limiting aspect of FIG. 26B, the second layup 2502_{b} can be unitized to the first layup 2502ₐ about bond line 1. However, it is important that the inner surfaces of the first and second layups 2502ₐ, 2502_{b} remain mechanically separable, as will be evident in FIG. 26C. According to the non-limiting aspect of FIG. 26C, the mechanically separate ends of the first and second layups 2502ₐ, 2502_{b} (e.g., the end proximal to bond line 2) are folded about a scam 2712 formed via the unitization process about bond line 1. For example, the mechanically separable end of the first layup 2502ₐ is folded about the seam 2512, over the LED bank 2520 and the mechanically separable end of the second layup 2502_{b} is folded about the seam 2512, below the LED bank 2520.

Once the first and second layups 2502ₐ, 2502_{b} are properly folded above and below the LED bank 2520, the first and second layups 2502ₐ, 2502_{b} of FIG. 26C can be unitized about bond line 2 on the opposite of the LED bank 2520 relative to bond line 1. The first and second layups 2502ₐ, 2502_{b} can then be unitized about the remaining edges to form flanged seal 2508 (FIG. 26A), thereby forming a sealed perimeter of a fluid-fillable portion 2510, that defines a cavity around the non-fluid-fillable portion 2510_{b} containing the LED bank 2520. Once again, it is important that the inner surfaces of the first and second layups 2502ₐ, 2502_{b} remain mechanically separable through unitization about bond line 2 and the rest of the seal 2508 (FIG. 26A), such that a cavity formed between the first and second layups 2502ₐ, 2502_{b} can expand about non-fluid-fillable portion 2510_{b} containing the LED bank 2520 when fluid is introduced to the cavity. Notably, at least one part of the non-fluid fillable portion 2510_{b} of the circuit 2500—shown here with the traces 2504 terminated in vias 2506—may remain external to the cavity of the fluid-fillable portion 2510ₐ, in this example forming an integrated “tongue-like” appendage as shown.

Similar to the partially-fluid fillable circuit 2200 of FIG. 22, the partially fluid-fillable circuit 2500 of FIG. 25 can include a substantially contained geometry, since at least a part of the non-fluid-fillable portion 2510_{b} with the LED bank 2520 traverses through the cavity of the fluid-fillable portion 2510ₐ and is completely encompassed by the fluid-fillable portion 2510ₐ. Accordingly, a portion of the traces 2204 and electronic components, such as the LED bank, arc also encompassed by the fluid-fillable portion 2210ₐ of the partially fluid-fillable circuit 2200. It shall be appreciated that other electronic components (e.g., a power source, a microprocessor, a logic-based controller, a transceiver, an electrode, a LED bank, a pump etc.) can be mounted to the non-fluid-fillable portion 2510_{b} such that the partially fluid-fillable circuit 2500 can be used as a midsole of a shoe, a personal massage device, a recreational ball, and/or a protective padding, all of which could benefit from the contained geometry and can be selectively inflated and/or deflated according to user preference and/or intended application

According to some non-limiting aspects, the LED bank 2520 of the partially fluid-fillable circuit 2500 of FIG. 25 can be communicably coupled to a microprocessor configured to receive signals from the traces 2504. The traces 2504 can be configured as a strain sensor of the partially fluid-fillable circuit 2500. As such, the microprocessor can determine a strain applied to the partially fluid-fillable circuit 2500 based, at least in part, on the signals received from the traces 2504. The microprocessor can transmit signals to the LED bank 2520 that are associated with the determined strain. Accordingly, the LED bank 2520 can illuminate a pattern, quantity, and/or color of LEDs that corresponds to strains applied to the partially fluid-fillable circuit 2500, as determined by the microprocessor. For example, the LED bank 2520 may illuminate more LEDs when a greater strain is applied to the traces 2504 as more pressure is applied to the partially fluid-fillable circuit 2500.

Alternately and/or additionally, the LED bank 2520 of the partially fluid-fillable circuit 2500 of FIG. 25 may illuminate different color LEDs, wherein each color is associated with a magnitude of strain applied to the traces 2504 in response to pressure applied to the partially fluid-fillable circuit 2500 (e.g., green associated with low strains, yellow associated with medium strains, red associated with high strains, etc.). In other words, the partially fluid-fillable circuit 2500 can be implemented for any application that would benefit from visual feedback associated with a physical condition of the fluid-fillable portion 2502ₐ, such as a wearable article (e.g., a joint monitoring sleeve, footwear, sportswear, etc.) exoskeletons, robotics, etc.

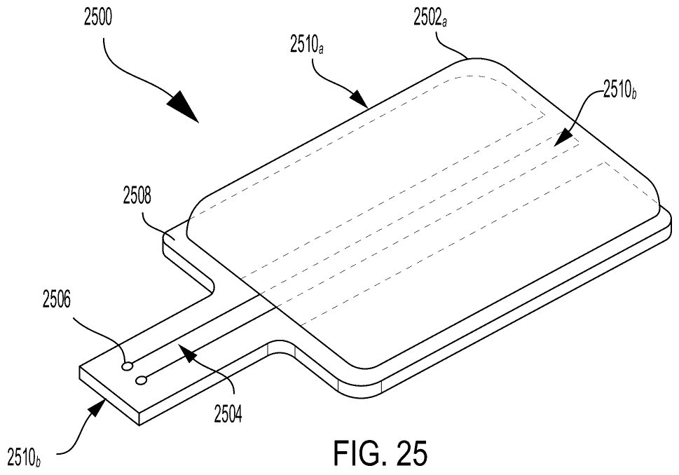

Referring now to FIG. 27, another partially fluid-fillable circuit 2700 is depicted in accordance with at least one non-limiting aspect of the present disclosure. The partially fluid-fillable circuit 2700 of FIG. 27 integrates several of the ideas previously discussed throughout the present disclosure. According to the non-limiting aspect of FIG. 27, the partially fluid-fillable circuit 2700 can include a first layup 2702ₐ mechanically coupled to a second layup 2702_{b} via a seal 2708. However, according to the non-limiting aspect of FIG. 27, the partially fluid-fillable circuit 2700 can include a plurality of fluid-fillable portions 2710ₐ, 2710_{c}, 2710ₑ mechanically coupled via a plurality of non-fluid-fillable portions 2710_{b}, 2710_{d}. Although the partially fluid-fillable circuit 2700 of FIG. 27 includes three fluid-fillable portions 2710ₐ, 2710_{c}, 2710ₑ and two non-fluid-fillable portions 2710_{b}, 2710_{d}.

According to the non-limiting aspect of FIG. 27, the first layup 2702ₐ and second layup 2702_{b} of the partially fluid-fillable circuit 2700 can include a network of traces 2704 formed from deformable conductors, electrical interconnects 2706, and/or electronic components that traverse the plurality of fluid-fillable portions 2710ₐ, 2710_{c}, 2710ₑ and plurality of non-fluid-fillable portions 2710_{b}, 2710ₑ. Once again, the first layup 2702ₐ, and second layup 2702_{b} of FIG. 27 can be configured similar to the layup 1702 of FIG. 17. Likewise, the deformable conductor of the traces 2704 of FIG. 27 and electrical interconnects 2706 can be configured similar to the deformable conductor of the traces 1704 and vias 1706 of FIG. 17, respectively.

Although the fluid-fillable portions 2710ₐ, 2710_{b}, 2710_{c} of FIG. 27 define cavities of substantially rectangular shape, the layups 2702ₐ, 2702_{b} can be alternately configured such that the cavities defined by the fluid-fillable portions 2710ₐ, 2710_{b}, 2710ₑ can include any desired volume and/or geometry. Accordingly, it shall be appreciated that, according to other non-limiting aspects, any of the fluid-fillable portions 2710ₐ, 2710_{c}, 2710ₑ can define cavities of varying volumes and/or shapes (e.g., circular, spherical, hexagonal, rectangular, triangular, etc.) depending on user preference and/or intended application. According to other non-limiting aspects, the fluid-fillable portions 2710ₐ, 2710_{c}, 2710ₑ of the partially fluid-fillable circuit 2700 can be arranged in a tessellated pattern. In other words, the number and geometric configuration of the plurality of fluid-fillable portions 2710ₐ, 2710_{c}, 2710ₑ and the plurality of non-fluid-fillable portions 2710_{b}, 2710ₑ can be specifically configured depending on user preference and/or intended application.

Upon assembly, the fluid-fillable portions 2710ₐ, 2710_{c}, 2710ₑ of the partially fluid-fillable circuit 2700 can be inflated using several methods of inflation, including those discussed in reference to FIG. 18. For example, the first layup 2702ₐ and/or the second layup 2702_{b} can include at least a portion fabricated from a microlayer membrane that can accommodate a needle, an inflation nozzle, an inflation electrode, or another inflation device during inflation but can be scaled, e.g., via the application of radio frequency energy or heat and/or pressure once a desired degree of inflation is achieved. However, according to other non-limiting aspects, a valve assembly can be mechanically coupled to the circuit 2700 of FIG. 27, and fluid can be selectively introduced and/or removed from the internal cavities of the fluid-fillable portions 2710ₐ, 2710_{c}, 2710ₑ via the valve assembly. According to some non-limiting aspects, the partially fluid-fillable circuit 2700 can include conduits positioned on the non-fluid-fillable portions 2710_{b}, 2710ₑ, wherein the conduits (not shown) can be configured to establish fluid communication between the plurality of fluid-fillable portions 2710ₐ, 2710_{c}, 2710ₑ. Accordingly, a fluid can be transported via the conduits to and from each of the plurality of fluid-fillable portions 2710ₐ, 2710_{c}, 2710ₑ. This can be particularly useful in non-limiting aspects wherein the partially fluid-fillable circuit 1700 is coupled to a source of fluid (e.g., a pump).

As such, the partially fluid-fillable circuit 2700 of FIG. 27 can realize all of the benefits described in reference to the circuit 1700 of FIGS. 17 and 18, including a more precise integration into a housing, the particular placement of electronic components, and an overall enhanced comfort for a user when integrated into a wearable article. More specifically, the partially fluid-fillable circuit 2700 can be specifically configured such that the partially fluid-fillable circuit 2700 can be integrated within a desired housing (e.g., a shoe, a joint monitoring sleeve, a robotic skeleton, etc.). The number and shape of fluid-fillable portions 2710ₐ, 2710_{c}, 2710ₑ can be varied to promote comfort and/or accommodate for space constraints, accordingly.

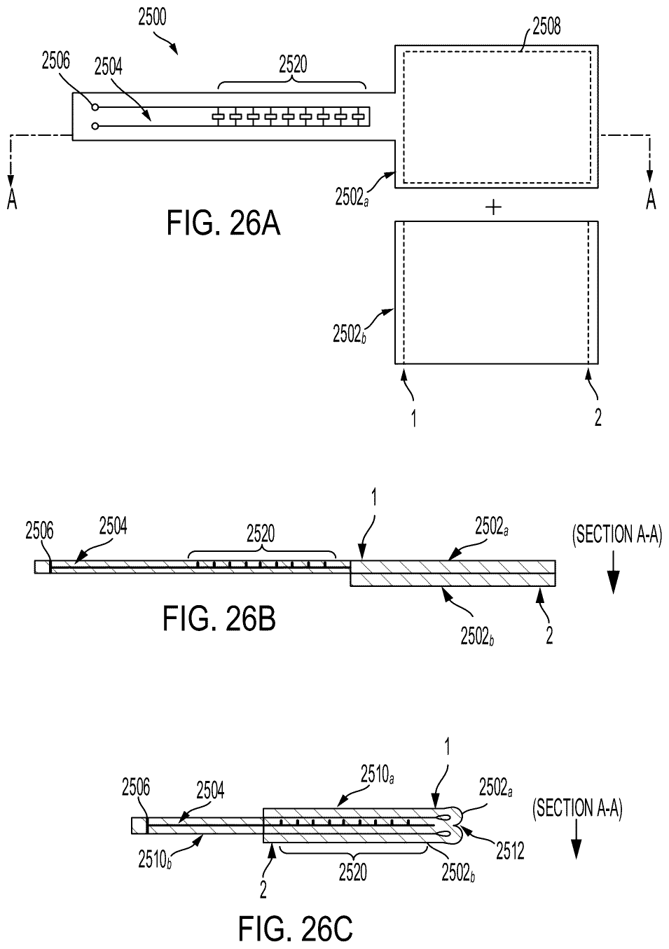

Referring now to FIG. 28A, a top view of the first layup 2702ₐ of the partially fluid-fillable circuit 2700 of FIG. 27 is depicted in accordance with at least one non-limiting aspect of the present disclosure. According to the non-limiting aspect of FIG. 28A, the first layup 2702ₐ can include a number of electrical structures, such as traces 2704ₐ formed from deformable conductors and/or electronic components, etc. that traverse the plurality of fluid-fillable portions 2710ₐ, 2710_{c}, 2710ₑ (FIG. 27) and plurality of non-fluid-fillable portions 2710_{b}, 2710ₑ (FIG. 27). For example, portions of the traces 2704 can be configured as a strain sensor, a pressure sensor, an antenna circuit, and/or a proximity sensor of the partially fluid-fillable circuit 2700. According to some non-limiting aspects, the traces 2704ₐ on the first layup 2702ₐ can be electrically coupled to electronic components (e.g., a power source, a microprocessor, a logic-based controller, a transceiver, an electrode, a LED bank, a pump etc.) mounted on the first layup 2702ₐ.

Moreover, the traces 2704ₐ on the first layup 2702ₐ of FIG. 28A can be electrically coupled to one or more vias 2706ₐ on the first layup 2702_{a,}which establish electrical communication between the traces 2704ₐ on the first layup 2702ₐ and the traces 2704_{b} on the second layup 2702_{b}. For example, according to the non-limiting aspect of FIG. 28B, traces 2704_{b} of the second layup 2702_{b} can be configured as a bus circuit for transporting electrical power to the traces 2704ₐ and/or any electronic components on the first layup 2702ₐ. Accordingly, the second layup 2702_{b} can be electrically coupled to a power source (not shown) and configured to transport electrical power and current throughout the partially fluid-fillable circuit 2700 of FIG. 27 via the traces 2704_{b} on the second layup 2702_{b}, through the electrical vias 2706ₐ, 2706_{b}, and through the traces 2704ₐ on the first layup 2702ₐ. Accordingly, the partially fluid-fillable circuit 2700 of FIG. 27 can perform any of the functions described in reference to any of the aforementioned circuits as the plurality of fluid-fillable portions 2710ₐ2710_{c}, 2710ₑ (FIG. 27) are selectively inflated and deflated.

As depicted in FIGS. 28A and 28B, the first layup 2702ₐ can have substantially the same geometric configuration as the second layup 2702_{b}, such that features of the first layup 2702ₐ can be aligned with corresponding features of the second layup 2702_{b}, when the first layup 2702ₐ is stacked on top of the second layup 2702_{b}. However, according to other non-limiting aspects, the first layup 2702ₐ can have a different geometric configuration than the second layup 2702_{b}, as long as features of the first layup 2702ₐ can be aligned with corresponding features of the second layup 2702_{b}, when the first layup 2702ₐ is stacked on top of the second layup 2702_{b}. Accordingly, when the first layup 2702ₐ is stacked on top of the second layup 2702_{b}, electrical connections between traces 2704ₐ, 2704_{b} and/or electronic components can be established via the electrical vias 2706ₐ, 2706_{b} and the first layup 2702ₐ can be unitized to the second layup 2702_{b} about the flanged seal 2708. Accordingly, inner surfaces of the first and second layups 2702ₐ, 2702_{b} can define cavities along with the flanged seal 2708, such that the plurality of fluid-fillable portions 2710ₐ, 2710_{c}, 2710ₑ (FIG. 27) can be filled with a fluid. Conversely, the non-fluid-fillable portions 2710_{b}, 2710_{c} (FIG. 27) can be unitized such that they do not define cavities and thus, cannot be filled with a fluid.

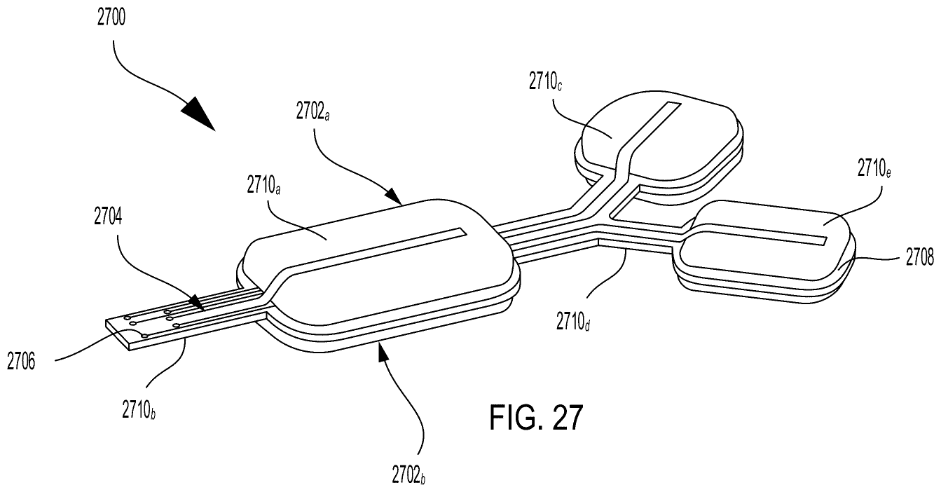

Referring now to FIGS. 29A and 29B, cross-sectioned views of one of the fluid-fillable portions 2710ₐ of the partially fluid-fillable 2700 circuit of FIG. 27, in accordance with at least one non-limiting aspect of the present disclosure. Specifically, FIGS. 29A and 29B depict the multi-layered construction of the first and second layups 2702ₐ, 2702_{b}, at a location where the fluid-fillable portion 2710ₐ transitions to the unitized, non-fluid-fillable portions 2710_{b}, 2710_{d}. According to the non-limiting aspect of FIGS. 29A and 29B, each of the first layup 2702ₐ and the second layup 2702_{b} can include a substrate layer 2719 on which a deformable conductor that forms the traces 2704 can be deposited, and an encapsulation layer 2717. However, according to some non-limiting aspects, either the first layup 2702ₐ or the second layup 2702_{b} can include a stencil layer that includes channels in which the deformable conductor can be deposited, thereby providing a more robust structural formation of the traces 2704.

Notably, FIGS. 29A and 29B further depict how the traces 2704 can be routed through the first and second layups 2702ₐ, 2702_{b} and how vias 2706, and other electrical interconnects, can be used to establish electrical communication between the traces 2704 and/or electronic components (e.g., a power source, a microprocessor, a logic-based controller, a transceiver, an electrode, a LED bank, a pump etc.) of the first and second layups 2702ₐ, 2702_{b}. Accordingly, the traces 2704 of the second layup 2702_{b} that are configured as a bus circuit can transport electrical power to the traces 2704 and/or any electronic components of the first layup 2702ₐ, when the bus circuit is electrically coupled to a power source (not shown). Of course, according to other non-limiting aspects, the traces 2704 of the first layup 2702ₐ can be configured as a bus circuit and the traces 2704 on the second layup 2702_{b} can be configured as a strain sensor, a pressure sensor, an antenna circuit, and/or a proximity sensor of the partially fluid-fillable circuit 2700. Thus, it shall be appreciated that the circuit 2700 configuration of FIGS. 27, 28A, and 28B is only exemplary, and that the partially fluid-fillable circuit 2700 can be alternately configured depending on user preference and/or intended application.

It shall be further appreciated that one or more vias can be implemented in any of the aforementioned partially-fluid-fillable circuits to convey currents and signals from a particular trace, between various layups, and/or to an electronic component (e.g., a power source, a microprocessor, a logic-based controller, a transceiver, an electrode, a LED bank, a pump etc.) mechanically coupled to the partially-fluid-fillable circuit. According to some non-limiting aspects, this can be accomplished during the production of the layup. Vias can be formed by removing material (e.g., via a laser cutting process, machining, punching, etc.) from one or more layers (e.g., substrate layer, encapsulation layer, etc.) of the layup and subsequently filling the newly-formed vacancy with a trace material, such as magnesium or a magnesium-based compound. Vias can be formed in desired locations during production or alternately, after the layup has been assembled such that the assembled circuit can be coupled to other layups to create a larger assembly. Accordingly, vias can be formed either before or after the circuit is filled, depending on user preference and/or intended application.

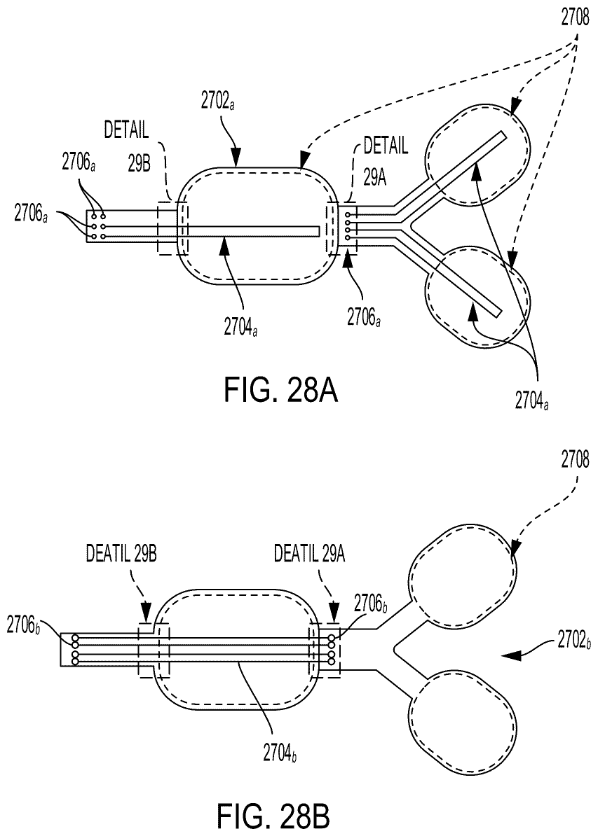

Referring now to FIGS. 30A and 30B, one implementation of another partially fluid-fillable circuit 3000 is depicted in accordance with at least one non-limiting aspect of the present disclosure. According to the non-limiting aspect of FIGS. 30A and 30B, the partially fluid-fillable circuit 3000 can be constructed similar to any of the partially fluid-fillable circuits disclosed herein. For example, the partially fluid-fillable circuit 3000 can include a plurality of traces 3004 formed from a deformable conductor and various portions of traces 3004 of various portions 3010_{a-f} of the partially fluid-fillable circuit 3000 can be electrically coupled via one or more electrical vias 3006. The partially fluid-fillable circuit 3000 can be constructed from layups configured similar to the layup 1702 of FIG. 17. Likewise, the deformable conductor of the traces 3004 of FIG. 30 and electrical vias 3006 can be configured similar to the deformable conductor of the traces 1704 and vias 1706 of FIG. 17, respectively.

Specifically, the partially fluid-fillable circuit 3000 can include a fluid-fillable portion 3010ₐ and a plurality of non-fluid fillable portions 3010_{b-f} arranged such that the partially fluid-fillable circuit 3000 can be properly installed within a housing 3020. According to the non-limiting aspect, of FIG. 30B, the housing 3020 can be a shoe. For example, the fluid-fillable portion 3010ₐ of the partially fluid-fillable circuit 3000 can form at least a portion of the midsole of a shoe, for example by being over-molded with foam to yield a midsole 3024 of the shoe-shaped housing 3020 and the non-fluid-fillable portions 3010_{b-f} of the partially fluid-fillable circuit 3000 can be laminated and/or otherwise adhered to various portions of an upper portion 3022 of the shoe-shaped housing 3020. Alternatively, the fluid-fillable portion 3010ₐ can form at least a portion of a midsole without being contained within foam, as is known in various commercially available shoe models. In such an alternative and non-limiting example, both the outsole and upper may be directly coupled to the fluid-fillable portion. According to other non-limiting aspects, the number and/or shape of the fluid-fillable portions 3010ₐ and non-fluid fillable portions 3010_{b-f} can be varied in accordance with user preference and/or intended application. For example, the number and/or shape of the fluid-fillable portions 3010ₐ and non-fluid fillable portions 3010_{b-f} can be varied to promote comfort and/or accommodate for space constraints, accordingly.

For example, according to some non-limiting aspects, the non-fluid fillable portions 3010_{b-f} can be used to measure strains in certain portions of the circuit, analyze fit, and/or adjust or suggest adjustments to the tension of a lacing system (e.g., auto-lacing, etc.) For example, some non-limiting aspects can include an auto-lacing application such as those disclosed in U.S. Pat. No. 10,743,620, titled AUTOMATED TENSIONING SYSTEM FOR AN ARTICLE OF FOOTWEAR, and granted Aug. 18, 2020, the disclosure of which is hereby incorporated by reference in its entirety. According to other non-limiting aspects, the lacing system can be passive, or otherwise a traditional lacing system. According to still other non-limiting aspects, the non-fluid fillable portions 3010_{b-f} can host electronic components on certain portions, such as processors, LED arrays or accelerometers, etc., or can be alternately configured to function as buses to a power source or to transmit signals from one portion hosting a sensor for some purpose, as provided above or other functions, to another portion hosting a processor or some other feedback device (e.g., haptic, displays, etc.).

According to some non-limiting aspects, the partially fluid-fillable circuit 3000 can include a LED bank or other electronic components (e.g., a power source, a microprocessor, a logic-based controller, a transceiver, an electrode, a LED bank, a pump etc.) configured to serve any functional and/or decorative purpose. For example, the partially fluid-fillable circuit 3000 can include a microprocessor and/or sensors configured to determine a pressure applied to the fluid-fillable portion 3010ₐ of the partially fluid-fillable circuit 3000. A user, via a pump or other means of inflating the fluid-fillable portion 3010ₐ could subsequently adjust the amount of cushioning provided by the fluid-fillable portion 3010ₐ, based on the determined pressure. According to other non-limiting aspects, the LED bank can provide a visual indication of how much is pressure presently applied to the fluid-fillable portion 3010ₐ.

According to still other non-limiting aspects, a transmitter or transmitter circuit of the partially fluid-fillable circuit 3000 can transmit an indication of the amount of pressure presently applied to the fluid-fillable portion 3010ₐ to a computing device (e.g., a smart phone, a tablet, a laptop computer, a desktop computer, etc.) of the user. According to still other non-limiting aspect, the user can transmit a command to the partially fluid-fillable circuit 3000 via a computing device to either inflate and/or deflate the fluid-fillable portion 3010ₐ of the partially fluid-fillable circuit 3000 thereby, controlling the amount of pressure applied to the fluid-fillable portion 3010ₐ in accordance with the user's preference. For example, according to some non-limiting aspects, the partially fluid-fillable circuit 3000 can be implemented as an electronically controlled bladder system, such as those disclosed in U.S. Pat. No. 9,066,558, titled ELECTRONICALLY CONTROLLED BLADDER ASSEMBLY, and granted Jun. 30, 2015, the disclosure of which is hereby incorporated by reference in its entirety.

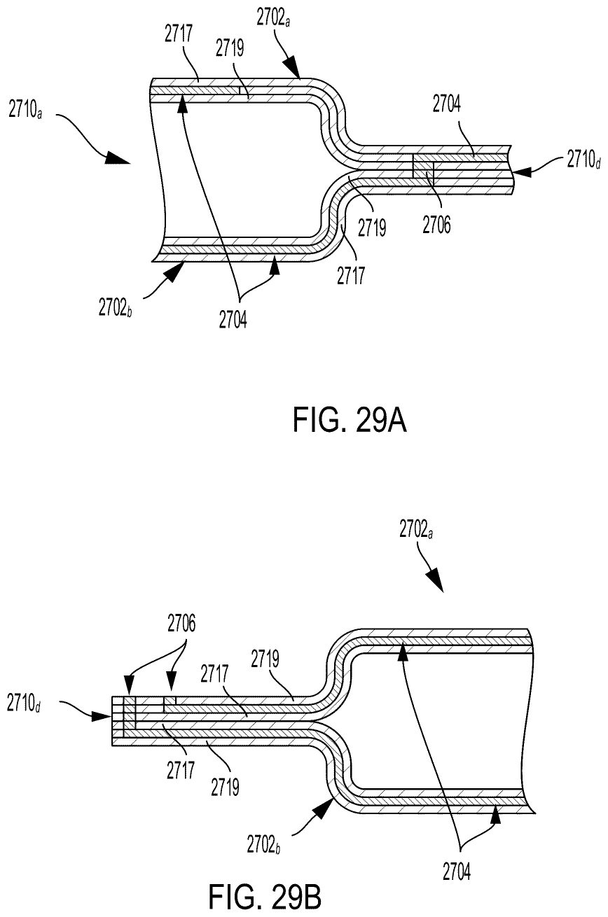

Referring now to FIG. 31, an assembly of the of partially fluid-fillable circuit 3000 of FIG. 30 is depicted in accordance with at least one non-limiting aspect of the present disclosure. According to the non-limiting aspect of FIG. 31, the partially fluid-fillable circuit 3000 can include a first layup 3002ₐ and a second layup 3002_{b}. As depicted in FIG. 31, the first layup 3002ₐ has a base layer 3026ₐ having a geometry that is substantially similar and corresponds with a base layer 3026_{b} of the second layup 3002_{b}. For example, both the base layers 3026ₐ, 3026_{b} of the first layup 3002ₐ and the second layup 3002_{b} appear to have an insole configuration or “foot-shape” when viewed from above. However, as depicted in FIG. 31, portions of the first layup 3002ₐ that will form the non-fluid-fillable portions 3010_{b-f} of the partially fluid-fillable circuit 3000 extend away from the insole-shaped base portion 3026ₐ of the first layup 3002ₐ. Accordingly, the base layer 3026ₐ of the first layup 3002ₐ can be positioned over the base layer 3026_{b} of the the second layup 3002_{b}, and the first and second layups 3002ₐ, 3002_{b} can be unitized about a flanged seal (not shown) configured to traverse a perimeter of the base portions 3026ₐ, 3026_{b}, thereby defining a cavity between inner surfaces of the base layers 3026ₐ, 3026_{b}. In other words, upon unitization, the base layer 3026ₐ of the first layup 3002ₐ and the base layer 3026_{b} of the second layup 3002_{b} can form the fluid-fillable portion 3010ₐ (FIG. 29) of the partially fluid-fillable circuit 3000. The fluid-fillable portion 3010ₐ (FIG. 29) can be selectively inflated and/or deflated using any of the techniques previously disclosed.

According to the non-limiting aspect of FIG. 31, both the first and second layups 3002ₐ, 3002_{b} can include a number of electrical structures, such as traces 3004ₐ, 3004_{b} formed from deformable conductors. As depicted in FIG. 31, the traces 3004ₐ, 3004_{b} can traverse portions of the base layers 3026ₐ, 3026_{b} and the plurality of non-fluid-fillable portions 3010_{b-f} of the first and second layups 3002ₐ, 3002_{b}. For example, portions of the traces 3004ₐ, 3004_{b} can be configured as a strain sensor, a pressure sensor, an antenna circuit, and/or a proximity sensor of the partially fluid-fillable circuit 3000. According to some non-limiting aspects, traces 3004ₐ, 3004_{b} on the first and second layups 3002ₐ, 3002_{b} can be electrically coupled to various electronic components (e.g., a power source, a microprocessor, a logic-based controller, a transceiver, an electrode, a LED bank, a pump etc.).

Moreover, the traces 3004ₐ on the first layup 3002ₐ of FIG. 31 can be electrically coupled to traces 3004_{b} on the second layup 3002_{b} via a plurality of electrical vias 3006ₐ, 3006_{b} on the first and second layup 3002ₐ, 3002_{b}, thereby establishing an electrical communication between the first layup 3002ₐ and the second layup 3002_{b}. The electrical vias 3006ₐ, 3006_{b} can be configured to establish different channels of electrical communication between various layers of the layups 3002ₐ, 3002_{b} and aggregated and/or arranged on the first and second layup 3002ₐ, 3002_{b} accordingly. For example, according to the non-limiting aspect of FIG. 31, the electrical vias 3006ₐ, 3006_{b} are arranged according to whether or not they establish electrical communication between a first layer 3028 of the layups 3002ₐ, 3002_{b} and a second layer 3030 of the layups 3002ₐ, 3002_{b}, thereby creating different channels of electrical communication throughout the partially fluid-fillable circuit 3000.

In other words, the electrical vias 3006ₐ, 3006_{b} of FIG. 31 can be configured to serve as contacts for electrically coupling various traces 3004ₐ, 3004_{b} of the first and second layups 3002ₐ, 3002_{b}. According to the non-limiting aspect of FIG. 31, this can include electrically coupling traces 3004ₐ positioned on the plurality of non-fluid-fillable portions 3010_{b-f} to traces 3004_{b} positioned on the second layup 3002_{b}. However, according to other non-limiting aspects, the traces 3004ₐ positioned on the plurality of non-fluid-fillable portions 3010_{b-f} can extend to the traces 3004ₐ positioned on the base portion 3026ₐ of the first layup 3026, which can then be to electrically coupled to traces 3004_{b} positioned on the second layup 3002_{b} via electrical vias 3006ₐ positioned on a heel portion of the base portion 3026ₐ of the first layup 3026.

According to some non-limiting aspects, traces 3004_{b} of the second layup 3002_{b} of FIG. 31 can be configured as a bus circuit for transporting electrical power to the traces 3004ₐ and/or any electronic components on the first layup 3002ₐ. Accordingly, the second layup 3002_{b} can be configured to transport electrical power and current throughout the partially fluid-fillable circuit 3000 via the traces 3004ₐ on the second layup 3002_{b}, through the electrical vias 3006ₐ, 3006_{b}, and through the traces 3004ₐ on the first layup 3002ₐ. Accordingly, the partially fluid-fillable circuit 3000 of FIG. 31 can perform any of the functions described in reference to any of the aforementioned circuits as the fluid-fillable portion 3010ₐ is selectively inflated and deflated.

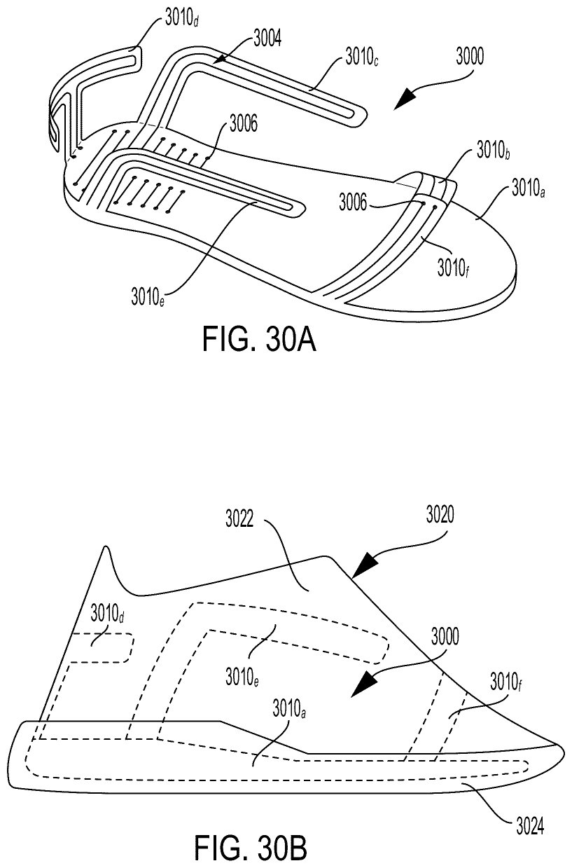

Referring now to FIG. 32, another implementation of another partially fluid-fillable circuit 3200 is depicted in accordance with at least one non-limiting aspect of the present disclosure. Specifically, the partially fluid-fillable circuit 3200 can include a fluid-fillable portion 3210ₐ and a plurality of non-fluid fillable portions 3210_{b-f} arranged such that the partially fluid-fillable circuit 3200 can optionally be installed within a housing 3220. According to the non-limiting aspect, of FIG. 30B, the housing 3020 can be a wearable article, similar to those disclosed in International Patent Application No. PCT/US2022/071012, titled DEVICES, SYSTEMS, AND METHODS TO MONITOR AND CHARACTERIZE THE MOTIONS OF A USER VIA FLEXIBLE CIRCUITS, and filed Mar. 7, 2022, the disclosure of which is hereby incorporated by reference in its entirety. For example, according to the non-limiting aspect of FIG. 32, the housing 3220 can be configured as a joint monitoring formed from elastic materials. However, according to other non-limiting aspects, the housing 3220 can form a sheet configured to be wrapped about a user's joint or appendage, such that the housing 3220 only forms a sleeve while in use.

Although the housing 3220 of FIG. 32 is worn on a user's leg and positioned about the user's knee, the housing 3220 can be alternately configured to be worn about any other joint, appendage, or other body part of the user (e.g., an elbow, a hand or foot, a digit, an ankle, a wrist, a knuckle, a shoulder, a vertebrae, and a hip, etc.). According to other non-limiting aspects, the wearable article can be a glove, similar to those disclosed in U.S. Provisional Patent No. 63/268,063, titled DEVICES, SYSTEMS, AND METHODS FOR GENERATING AND CORRELATING ELECTRICAL PARAMETERS TO THE PHYSICAL MOTIONS OF A USER, filed Feb. 15, 2022, the disclosure of which is hereby incorporated by reference in its entirety.

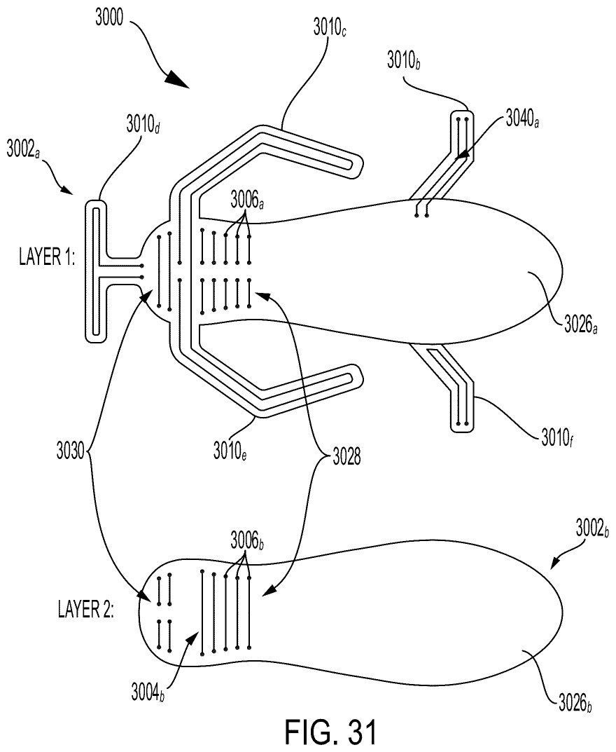

In further reference to FIG. 32, the partially fluid-fillable circuit 3200 can include two fluid-fillable portions 3210ₐ, 3210_{b} and a non-fluid-fillable portion 3210_{c} laminated and/or otherwise adhered to various portions of an upper portion 3222 of the housing 3220. According to the non-limiting aspect of FIG. 32, the non-fluid-fillable portion 3210_{c} can be positioned about the user's joint (e.g., a knee), a first fluid-fillable portion 3210ₐ can be positioned above the joint, and a second fluid-fillable portion 3210_{b} can be positioned below the joint. As will be described in further detail with reference to FIG. 33, the fluid-fillable portions 3210ₐ, 3210_{b} can define an fluid-fillable cavity that is annular and configured to traverse the user's appendage. However, according to other non-limiting aspects, the position, number and/or shape of the fluid-fillable portions 3210ₐ, 3210_{b} and non-fluid-fillable portions 3210_{c} can be varied in accordance with user preference and/or intended application. For example, the position, number and/or shape of fluid-fillable portions 3010ₐ, 3210_{b} and non-fluid-fillable portions 3210_{c} can be varied depending on the joint, appendage, or body part to be monitored, thereby promoting comfort and/or accommodating for space constraints.

According to the non-limiting aspect of FIG. 32, the two fluid-fillable portions 3210ₐ, 3210_{b} and the non-fluid-fillable portion 3210_{c} can include a number of traces 3204ₐ, 3204_{b}, 3204_{c} and/or electronic components 3205ₐ, 3205_{b}, 3207ₐ, 3207_{b} configured to monitor the joint, appendage, or body part of the user. The traces 3204ₐ, 3204_{b}, 3204_{c} and/or electronic components 3205ₐ, 3205_{b}, 3207ₐ, 3207_{b} can be electrically coupled via electrical interconnects such as electrical vias 3206ₐ, 3206_{b}, a previously discussed. According to the non-limiting aspect of FIG. 32, the traces 3204ₐ, 3204_{b}, 3204_{c} of the partially fluid-fillable circuit 3200 can be configured as a simple strain sensor. However, according to other non-limiting aspects, the traces 3204ₐ, 3204_{b}, 3204_{c} of the partially fluid-fillable circuit 3200 can be alternately and/or additionally configured as a pressure and/or proximity sensor, a bus circuit for transporting electrical power to various components, and/or an antenna circuit for transmissions, amongst other circuit types.

Still referring to FIG. 32, each of the two fluid-fillable portions 3210ₐ, 3210_{b} can include a number of electrodes 3207ₐ, 3207_{b}, e.g., EMG electrodes and/or an inertial measurement unit (“IMU”) 3205ₐ, 3205_{b} positioned on or about a cavity defined by the fluid-fillable portions 3210ₐ, 3210_{b}. For example, according to the non-limiting aspect of FIG. 32 the electrodes 3207ₐ, 3207_{b} can be positioned on an outer surface of a second layup 3202_{b} (FIG. 33) that defines the fluid-fillable portions 3210ₐ, 3210_{b} of the partially fluid-fillable circuit 3200. However, the outer surface of the second layup 3202_{b} (FIG. 33) may form an inner surface of the housing 3220 that interfaces with the user's skin. Accordingly, the electrodes 3207ₐ, 3207_{b} can be placed in electrical contact with the user's skin, such that the electrodes 3207ₐ, 3207_{b} can monitor and/or stimulate a user's joint, appendage, or body part at a particular location. Accordingly, as the fluid-fillable portions 3210ₐ, 3210_{b} of the partially fluid-fillable circuit 3200 are selectively inflated and/or deflated, the electrodes 3205ₐ, 3205_{b} can be biased against the user.

Conversely, according to the non-limiting aspect of FIG. 32, the IMU's 3205ₐ, 3205_{b} can be positioned on an outer surface of a first layup 3202ₐ (FIG. 33) that defines the fluid-fillable portions 3210ₐ, 3210_{b}, of the partially fluid-fillable circuit 3200. However, the outer surface of the first layup 3202ₐ (FIG. 33) may form an outer surface of the housing 3220 that does not interface with the user's skin. Accordingly, the IMU's 3205ₐ, 3205_{b} may not physically contact the user's skin and thus, will not cause discomfort when the user is wearing the housing 3220 about their joint, appendage, or body part. Of course, according to other non-limiting aspects, the electronic components 3205ₐ, 3205_{b}, 3207ₐ, 3207_{b} can be positioned in other locations in accordance with user preference and/or intended application. Moreover, according to other non-limiting aspects, the partially fluid-fillable circuit 3200 of FIG. 32 can include other electronic components (e.g., a power source, a microprocessor, a logic-based controller, a transceiver, a LED bank, a pump etc.) configured to serve other functional and/or decorative purposes. For example, according to some non-limiting aspects, the partially fluid-fillable circuit 3200 can further include an LED bank configured to provide a visual indication of a magnitude of strain applied to one or more of the traces 3204ₐ, 3204_{b}, 3204_{c} in response to pressure applied to the partially fluid-fillable circuit 3200, as previously discussed.

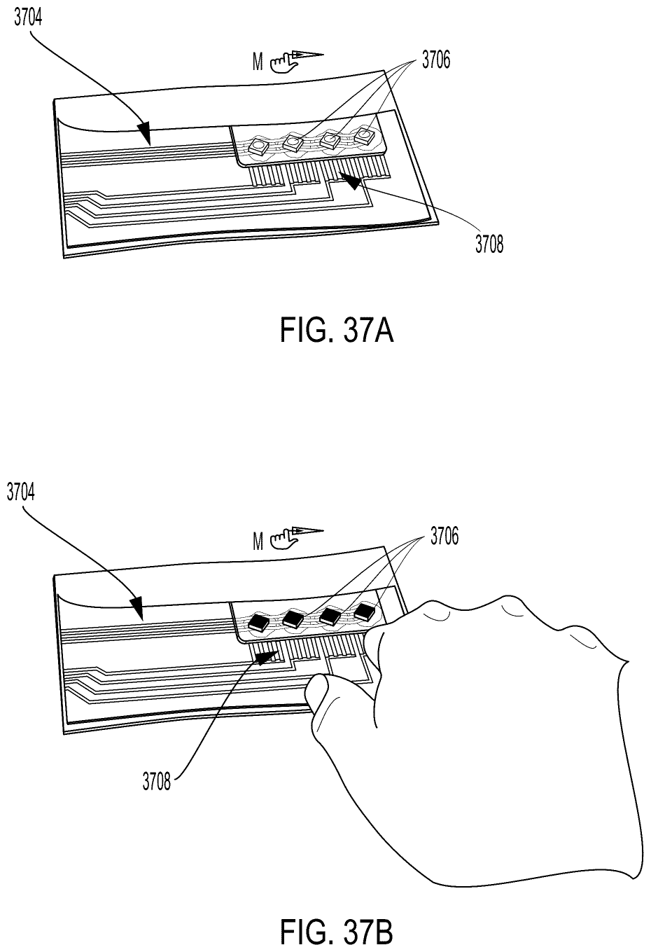

According to some non-limiting aspects, the one or more of the traces 3204ₐ, 3204_{b}, 3204_{c} can be implemented as part of a capacitive touch interface. For example, referring now to FIGS. 37A and 37B, a capacitive touch interface 3700 configured for use with any of the aforementioned partially-fluid-fillable circuits is depicted in accordance with at least one non-limiting aspect of the present disclosure. According to the non-limiting aspect of FIG. 37A and 37B, the capacitive touch interface 3700 can include a flexible circuit formed from traces 3704 deformable conductors. The traces 3704 can be coupled to an array of LEDs 3706, which can also be electrically coupled to another arrangement of deformable traces 3708 configured to function a capacitive sensor, for example, comprising coils. The traces 3708 can be embedded within a layup and/or a fabric from which the circuit or interface 3700 is constructed. As such, if a user were to interact with one or more of the traces 3708 configured to function as a capacitive sensor, one or more LEDs 3706 of the array can be illuminated, as depicted in FIGS. 37A and 37B. In FIG. 37A, the user performs motion M by sliding their finger across the traces 3708 configured to function as a capacitive sensor and thus, all of the LEDs 3706 of the array have illuminated, as depicted in FIG. 37B. Alternately and/or additionally, the use can press their finger lightly to activate one or more LEDs 3706, selectively.

Still referring to FIGS. 37A and 37B, it shall be appreciated that a capacitive touch interface 3700 can be implemented to receive a user input. For example, by pressing the traces 3708 configured to function a capacitive sensor, the user can provide signals to and from an electronic component communicably coupled to the capacitive touch interface 3700. For example, according to some non-limiting aspects, the electronic component can include a microprocessor configured to selectively inflate and/or deflate a fluid-fillable portion of the partially-fluid-fillable circuit in response to user inputs provided via the various traces 3708 of the capacitive touch interface 3700. Additionally, the array of LEDs 3706 can provide a visual indicia as to the degree of inflation of a fluid-fillable portion of the partially-fluid-fillable circuit, as various LEDs 3706 can be illuminated in accordance with various user inputs. Of course, according to other non-limiting aspects, pressing the traces 3708 of the capacitive touch interface 3700 can be configured to transmit any other command to any other electronic component communicably coupled to the partially-fluid-fillable circuit.

Referring now to FIG. 33, a perspective view of a fluid-fillable portion 3210 of the partially fluid-fillable circuit 3200 of FIG. 32 is depicted in accordance with at least one non-limiting aspect of the present disclosure. According to the non-limiting aspect of FIG. 33, the fluid-fillable portion 3210 can be constructed from a first layup 3202ₐ and a second layup 3202_{b}. The first and second layups 3202ₐ, 3202_{b} can include a multi-layer construction similar to those discussed in reference to the other non-limiting aspects disclosed herein. However, according to the non-limiting aspect of FIG. 33, the first and second layups 3202ₐ, 3202_{b} can have substantially corresponding rectangular geometries such that, when the first layup 3202ₐ is positioned over the second layup 3202_{b} the first and second layups 3202ₐ, 3202_{b} can be unitized about a perimeter of the desired fluid-fillable cavity 3215 via known processes (e.g., heat, pressure, RF energy, adhesive, etc.), thereby creating a seal 3208. Along with an inner surface of the first and second layups 3202ₐ, 3202_{b}, the seal 3208 can define the cavity 3215 between the first and second layups 3202ₐ, 3202_{b}, Furthermore, the fluid-fillable portion 3210 can include one or more traces 3204 that traverse either or both of the first and second layups 3202ₐ, 3202_{b} about the cavity 3215. As described in reference to FIG. 32, the electrodes 3217 can be mounted to an external surface of the second layup 3202_{b}. Accordingly, as the fluid-fillable portion 3210 of the partially fluid-fillable circuit 3200 is inflated and/or deflated, the electrodes 3205 can be biased against the user.

According to other non-limiting aspects, once the first and second layups 3202ₐ, 3202_{b} of FIG. 33 are unitized and the cavity 3215 is defined, the unitized first and second layups 3202ₐ, 3202_{b} can be folded and joined at the ends such that the first and second layups 3202ₐ, 3202_{b} collectively define a fluid-fillable portion 3210 of annular configuration. The folded first and second layups 3202ₐ, 3202_{b} can be subsequently unitized about joint 3209 via known processes (e.g., heat, pressure, RF energy, adhesive, etc.), thereby forming a butt or a lap joint. The annular configuration depicted in FIG. 33 can be beneficial as it enables the fluid-fillable portion 3210 to be worn about an appendage of the user (e.g., a leg, as depicted in FIG. 32). However, according to non-limiting aspects wherein the housing 3220 (FIG. 20) is alternately configured to function as a wrap, joint 3209 may be unnecessary, and the unitized first and second layups 3202ₐ, 3202_{b} can be placed into an annular configuration by the user and maintained there via alternate means (e.g., friction, a clip, a tape, an adhesive, etc.). According to still other non-limiting aspect of FIG. 33, the partially-fluid-fillable circuit 3200 can include a single layup (e.g., either of layups 3202ₐ, 3202_{b}) having a rectangular form and unitizing it about its free ends (e.g., via a lap joint, etc). The single, unitized layup can then create a ring, which can be folded, similar to a toque, and further unitized about its annulus to form the fluid-fillable cavity 3215. Thus, a single layup can be used to form the fluid-fillable portion 3210 and a non-fluid-fillable portion 3213 can be mechanically can electrically coupled via one or more electrical vias 3206.

In further reference to FIG. 33, the cavity 3215 can be defined between the first and second layups 3202ₐ, 3202_{b}, which can contain traces 3204 formed from deformable conductors that are electrically coupled to the electrodes 3205. The traces 3204 can traverse one or more layers of the first and second layups 3202ₐ, 3202_{b} and can be electrically coupled to the rest of the partially fluid-fillable circuit 3200 via one or more electrical vias 3206. According to the non-limiting aspect of FIG. 33, the one or more electrical vias 3206 can be positioned on a non-fluid-fillable portion 3213 of the circuit 3200 configured to establish electrical communication between the fluid-fillable portion 3210 and the rest of the circuit 3200, as depicted in FIG. 32. Additionally, the fluid-fillable portion 3210 of FIG. 33 can include a conduit 3211 configured to establish fluid communication between the fluid-fillable cavity 3215 of the fluid-fillable portion 3210 and a fluid source, other electronic components (e.g., a pump) and/or other fluid-fillable portions of the partially-fluid-fillable circuit 3200. According to some non-limiting aspects, the conduit 3211 can include a valve assembly configured to selectively permit the transmission of fluids to and/or from the cavity 3215.

According to some non-limiting aspects, the fluid-fillable portion 3210 of FIGS. 32 and 33 can include an electromagnetic electronic component (e.g. an electrode, an electromagnet, a ferromagnetic component, etc.) mechanically coupled to a first portion of the cavity 3215 and a corresponding second electromagnetic component or layer (e.g., a conductive layer, a second electromagnet, a ferromagnetic layer, etc.) mechanically coupled to a second portion of the cavity 3215. Furthermore, the cavity 3215 can be filled with a dielectric fluid. For example, the electromagnetic component can include an electrode 3207 and the second electromagnetic component can include a conductive layer. A potential or voltage can be applied by the partially fluid-fillable circuit 3200 to the electrode 320. In response to the applied potential or voltage, the electrode 3207 can be configured to bias the first portion of the first fluid fillable cavity towards and/or away from the second fluid fillable portion of the cavity. Such biasing can be caused by an electromagnetic attraction and/or an electromagnetic repulsion of the electrode 3207 relative to the conductive layer. In other words, the fluid-fillable portion 3210 of FIGS. 32 and 33 can be configured to function as a HASEL muscle. According to some non-limiting aspects, the fluid-fillable cavity 3215 can have multiple discrete chambers or volumes, effectively defining a cavity as at least one sealed, perimeter volume. According to still other non-limiting aspects, a conduit can be configured to otherwise penetrate the sealed, perimeter volume.

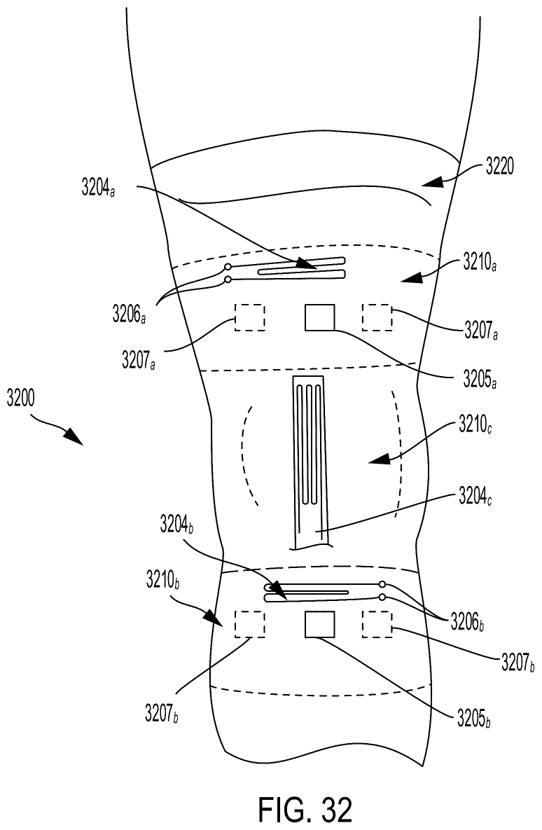

Referring now to FIG. 34, an alternate cavity 3215 configuration 3400 configured for use with the fluid-fillable portion 3210 of FIG. 33 is depicted in accordance with at least one non-limiting aspect of the present disclosure. According to the non-limiting aspect of FIG. 34, a first and second layup 3202ₐ, 3202_{b} (FIG. 33) can be alternately unitized such that one or more fluid fillable bladders 3415 are defined. Contrary to the annular cavity configuration of the fluid-fillable portion 3410 depicted in FIG. 33, a majority of the inner surfaces of the first and second layups 3202ₐ3202_{b} (FIG. 33) can be unitized, such that fluid is only introduced to the one or more fluid fillable bladders 3415. As described in reference to FIGS. 32 and 33, the electrodes 3215 can be mounted to an external surface of the second layup 3202_{b} (FIG. 33). However, according to the non-limiting aspect of FIG. 34 the electrodes can be mounted at a location on the second layup 3202_{b} (FIG. 33) that corresponds to the one or more fluid fillable bladders 3415. Accordingly, as the one or more fluid-fillable bladders 3415 are inflated and/or deflated, the electrodes 3207 can be biased against the user.

According to some non-limiting aspects, the fluid-fillable portion of FIG. 34 can include a conduit 3411. The conduit 3411 can be configured such that a non-fluid-fillable portion can still convey fluid and thus, remain in fluid communication with at least on fluid-fillable portion of the circuit. As such, the conduit 3411 can enable the conveyance of fluids between multiple fluid-fillable portions, even when said fluid-fillable portions are separated by non-fluid-fillable portions. Alternately and/or additionally, one or more conduits 3411 can be configured to establish fluid communication between a first fluid-fillable bladder 3415 and a fluid source, an electronic component (e.g., a pump) and/or other fluid-fillable portions of the partially-fluid-fillable circuit 3200. Additionally, the first and second layups 3202ₐ, 3202_{b} (FIG. 33) can be unitized to further define a fluid-fillable channel 3417 that establishes fluid communication between the first fluid-fillable bladders 3415 and a second fluid-fillable bladders 3415. Of course, the electrodes 3407 can remain in electrical communication via one or more traces 3404 of the first and second layups 3202ₐ, 3202_{b} (FIG. 33) formed from deformable conductors. According to the non-limiting aspect of FIG. 34, the electrodes 3407 can remain in electrical communication with other portions of the partially fluid-fillable circuit 3200 via one or more electrical vias 3406, as previously discussed.

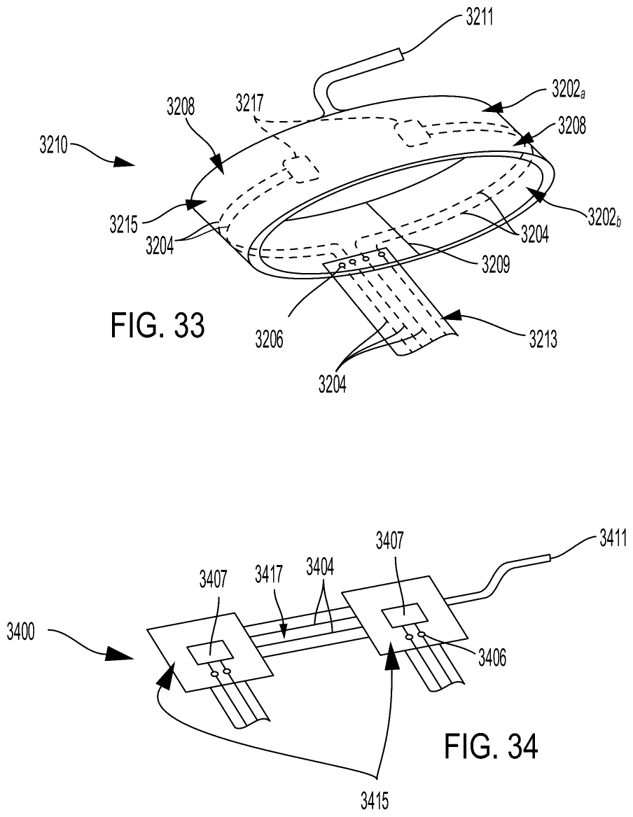

Referring now to FIG. 35, a plan view of a conduit 3515 configuration 3500 is depicted in accordance with at least one non-limiting aspect of the present disclosure. According to the non-limiting aspect of FIG. 35, the configuration 3500 can include one or more traces 3504 configured to traverse one or more sides of a conduit 3515. The conduit 3500 can be between at least two layers (e.g., substrate layer, encapsulation layer) of a layup. For example the conduit 3515 can be formed similar to the traces of the other non-limiting aspects disclosed herein. Assuming the layup utilizes a stencil layer, the conduit 3515 can be formed by the stencil layer but, instead of depositing a deformable conductor within a channel defined by the stencil layer, can be left empty such that a fluid can be contained by and traverse the channel upon encapsulation. Thus, the channel can become a conduit 3515 and not a trace of the circuit. Alternately, when the stencil layer and encapsulation layer of a layup are unitized to create the layup, a tooling can be deposited between the stencil layer and encapsulation layer in an area when the user intends on defining the conduit 3515. Accordingly, the stencil layer and the encapsulation layer will not be unitized about the tooling and the tooling can be removed, thereby defining the conduit 3515. According to still other non-limiting aspects, the an intermediate adhesive can be deposited between the stencil layer and the encapsulation layer, but omitted in an area where the user intends on defining the conduit 3515.

Moreover, the configuration 3500 of FIG. 35 can enable a fluid to traverse the conduit 3515 adjacent one or more traces 3504, which may run hot via the conveyance of signals, such as an electric current through a first portion 3520ₐ and a second portion 3520_{b} of a layup. As such, it shall be appreciated that the configuration 3500 of FIG. 35 can employ a cooling fluid through the conduit 3515, which can be used to cool the traces, the first portion 3520ₐ and the second portion 3520_{b}, the whole layup, and/or the overall assembly. This can prevent overheating and preserve the life of the circuit, inadvertent melting of the layups, and can enable the overall configuration 3500 and/or circuit to comply with system-level thermal requirements. For example, if the configuration 3500 were deployed in a wearable, the conduit 3515 can prevent the inadvertent burning of a user.

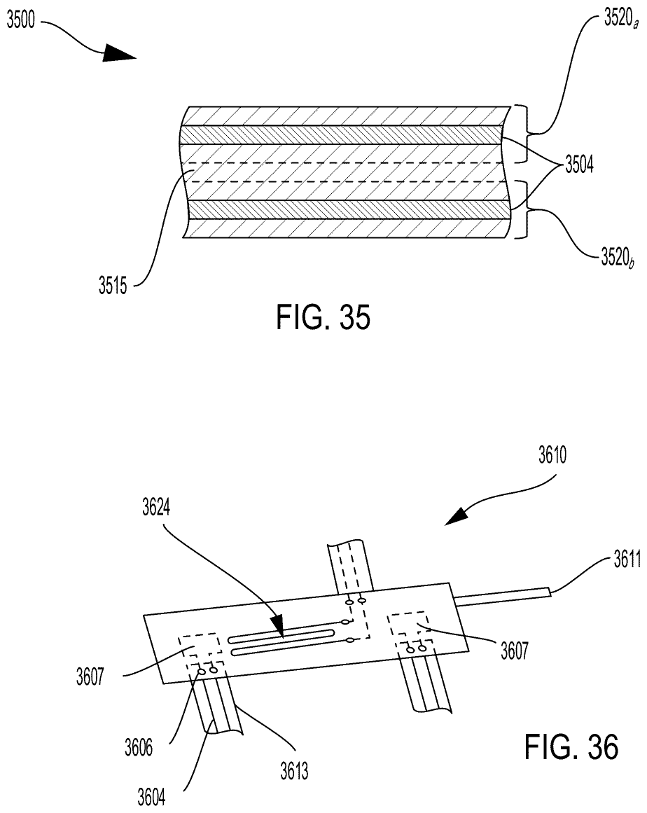

Referring now to FIG. 36, another fluid-fillable portion 3610 configured for use with the partially fluid-fillable circuit 3200 of FIG. 32 is depicted in accordance with at least one non-limiting aspect of the present disclosure. The fluid-fillable portion 3610 of FIG. 36 can either include an annular fluid-fillable cavity, such as the fluid-fillable cavity 3215 of FIG. 33, or one or more fluid-fillable bladders, such as the fluid-fillable bladders 3415 of FIG. 34. Moreover, the fluid-fillable portion 3610 can include one or more electrodes 3607 coupled to an external surface of a second layup, such as the second layup 3202_{b} of FIG. 33. The electrodes 3607 can be electrically coupled to other portions of the partially fluid-fillable circuit 3200 (FIG. 32) via traces 3604 formed from deformable conductors and/or electrical interconnects, such as electrical vias 3606, positioned on one or more non-fluid-fillable portions 3613. Likewise, the fluid-fillable portion 3610 of FIG. 36 can include a conduit 3611 configured to establish fluid communication between a fluid-fillable cavity of the fluid-fillable portion 3610 and a fluid source, an electronic component (e.g., a pump), and/or other fluid-fillable portions of the partially-fluid-fillable circuit 3200 (FIG. 32).

However, according to the non-limiting aspect of FIG. 36, the fluid-fillable portion 3610 can include a plurality of traces 3624 configured to function as a sensor. According to the non-limiting aspect of FIG. 36, the traces 3624 can be configured to function as a simple strain sensor. Alternatively, the traces 3624 can generate varying electrical parameters (e.g., an inductance, a resistance, a voltage drop, a capacitance, signal, an electromagnetic field, etc.) as an electrical current and/or potential is be applied to the circuit 3600 and the fluid-fillable portion 3610 is inflated, deflated, and/or otherwise deformed. However, according to other non-limiting aspects, the traces 3624 of the partially fluid-fillable circuit 3600 can be alternately and/or additionally configured as a pressure and/or proximity sensor, a bus circuit for transporting electrical power to various components, and/or an antenna circuit for transmissions, amongst other circuit types.

According to some non-limiting aspects, it may be preferable to compose the fluid-fillable circuits disclosed herein from alternate materials. For example, according to some non-limiting aspects, at least one layer of any of the layups described herein (e.g., first layup 102ₐ, second layup 102_{b}, etc.) can be composed of a silicone-based and/or rubber-like material, including thermoplastic polyurethanes, Silastic LC1000 silicone rubber, Momentive RTV-630 Blue, and/or polydimethylsiloxane (“PDMS”), amongst others. Such materials may provide mechanical or chemical properties that make them more suitable for some applications (e.g., high elongation or temperature resistance) and thus, may be particularly beneficial when implemented as a substrate layer and/or encapsulation layer for a layup when certain characteristics are needed for the final structure. In some examples, such properties could result in circuits that are more robust when washed repeatedly, which could be beneficial when the circuits disclosed herein are implemented via wearable devices. However, use of such materials may require alternate methods of manufacture. For example, whereas the previously disclosed materials may lend themselves to using pre-formed sheets in a lamination method of manufacture, a rubber-like material may utilize a casting, spraying, and/or molding-type method of manufacture. For example, it shall be appreciated that fluidic channels can be formed in a PDMS substrate using plasma activated bonding, as described by K. Choonee, R. R. A. Syms, M. M. Ahmad, and H. Zou in Post Processing of Microstructures by PDMS Spray Deposition, published in Volume 347 of the journal Sensors and Actuators A: Physical, which is scheduled to be published on Nov. 1, 2022.

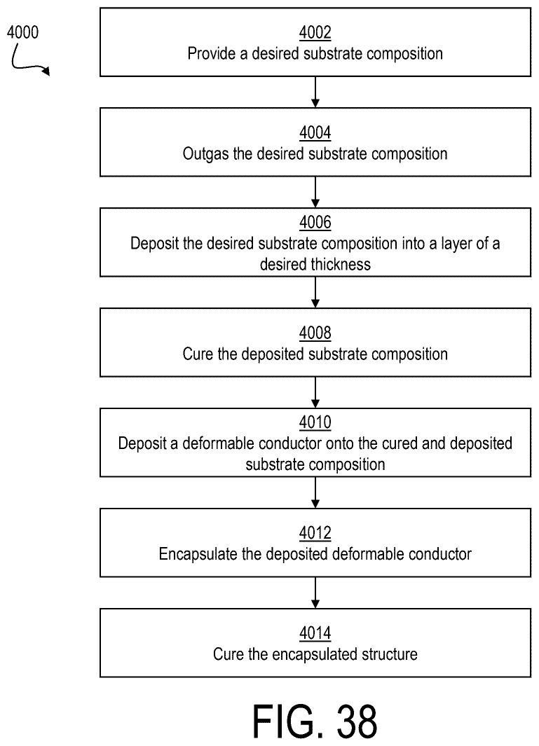

Referring now to FIG. 38, a flow chart of a method 4000 of manufacturing a substrate from which a fluid-fillable circuit can be produced is depicted in accordance with at least one non-limiting aspect of the present disclosure. Generally, the method 4000 of FIG. 38 can be used to produce at least one layer of at least one layup utilized in any of the fluid-fillable circuits disclosed herein from a silicone-based and/or rubber-like material. According to the non-limiting aspect of FIG. 38, the method 4000 can include providing 4002 a desired substrate composition. For example, the composition may require the provision and mixing of certain components. According to the non-limiting aspects wherein the substrate composition includes a Silastic LC1000 silicone rubber or a Momentive RTV-630 Blue, the substrate composition may require the provision of a Part A and a Part B, which need to be mixed according to the ratios specified in a corresponding data sheet to achieve the desired substrate composition. The method 4000 can further include outgassing 4004 the substrate composition. Since silicone-based and/or rubber-like materials can include a relatively high oil content compared to the alternate materials disclosed herein, such materials may require at least a minimum amount of outgassing. According to some non-limiting aspects, the outgassing 4004 can include a predetermined duration of exposure to a predetermined environment. For example, the predetermined environment can include a specified temperature range and, according to some non-limiting aspects, a vacuum. According to other non-limiting aspects, an oil-less (e.g., A-stage) PDMS may be used, within which most of the oils undergo a full reaction as the composition sets and thus, the oils are less likely to disperse within the uncured material as an impurity. Thus, the outgassing step 4004 can be optional, reduced, and/or omitted entirely, depending on the selected substrate composition.

In further reference to FIG. 38, the method 4000 can further include depositing 4006 the desired substrate composition into a layer of a desired thickness. According to the non-limiting aspect wherein the substrate composition includes a Silastic LC1000 silicone rubber, the deposition can include use of a screen-printing means to deposit the substrate composition onto a fabric such as Dyneema®, or any other suitable fabric. According to the non-limiting aspect wherein the substrate composition includes Momentive® RTV-630 Blue, the deposition can include pouring the substrate composition onto a flat surface and using a wiper blade, a roller, or other means until the substrate composition achieves a desired thickness. Upon achieving the desired thickness, the method 4000 can further include curing 4008 the deposited substrate composition and depositing 4010 a deformable conductor onto the substrate composition once it has cured. According to some non-limiting aspects, deposition of the deformable conductor can include a stencil layer. However, according to other non-limiting aspects, the deformable conductor can be deposited directly onto the substrate composition without the use of stencil layer as described in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, and filed Feb. 25, 2022, the disclosure of which is herein incorporated by reference in its entirety. Finally, the method 4000 can include encapsulating 4012 the deposited deformable conductor. According to some non-limiting aspects, the encapsulation layer can include the same silicone-based and/or rubber-like material as the substrate composition. As such, the method 4000 can include curing 4014 the encapsulated structure. It shall be appreciated that unitization can occur as the substrate composition and encapsulation layers are cured. However, according to some non-limiting aspects, one or more layers or portions thereof may utilize one or more of a primer, catalyzer, plasma and/or corona discharge surface treatment to enhance and/or accelerate the unitization during the curing step 4012.

According to some non-limiting aspects, vias may be necessary to ensure multi-layer connectivity is established throughout the layup. However, use of a silicone-based and/or rubber-like material to compose the layup and the method 4000 of FIG. 38 could complicate the production of vias. Therefore, according to some non-limiting aspects, the method 4000 can further include the creation of vias using a material removal process such as a laser ablation or etching to remove the silicone-based and/or rubber-like material in a desired geometry and location of the via, and subsequently filling the resulting via with a deformable conductor to establish connectivity between various components of the circuit. For example, upon curing or unitization of an encapsulation or substrate layer, one or more vias can be laser-etched or milled, such deformable conductors deposited on additional layers may be in electrical communication with deformable conductors in other layers. Accordingly, silicone-based and/or rubber-like materials can be used to produce multi-layer circuits, wherein the traces are routed vertically and horizontally when viewed in a cross-section.

Since the inventive principles of this patent disclosure can be modified in arrangement and detail without departing from the inventive concepts, such changes and modifications are considered to fall within the scope of the following claims. The use of terms such as first and second are for purposes of differentiating different components and do not necessarily imply the presence of more than one component.

Various aspects of the subject matter described herein are set out in the following numbered clauses:

Clause 1: A partially fluid-fillable circuit assembly, including a layup including: a substrate layer; a deformable conductor; and an encapsulation layer covering the deformable conductor; wherein a first portion of the layup includes a sealed perimeter that, along with at least one surface defined by the layup, defines a first fluid-fillable cavity, wherein a second portion of the layup is unitized and structurally distinguished from the cavity defined by the first portion of the layup.

Clause 2: The partially fluid-fillable circuit assembly according to clause 1, wherein at least one part of the second portion of the layup is external to the first fluid fillable cavity.

Clause 3: The partially fluid-fillable circuit assembly according to clauses 1 or 2, wherein at least a first part of the second portion of the layup is contained within the first fluid fillable cavity.

Clause 4: The partially fluid-fillable circuit assembly to any of clauses 1-3, wherein a second part of the second portion of the layup is external to the first fluid fillable cavity.

Clause 5: The partially fluid-fillable circuit assembly according to any of clauses 1-4, wherein the deformable conductor defines a first pattern of traces through the first portion of the layup.

Clause 6: The partially fluid-fillable circuit assembly according to any of clauses 1-5, wherein the deformable conductor defines a second pattern of traces through the second portion of the layup.

Clause 7: The partially fluid-fillable circuit assembly according to any of clauses 1-6, wherein an electronic component is coupled to the layup and electrically coupled to the first pattern of traces and the second pattern of traces.

Clause 8. The fluid-fillable circuit assembly according to any of clauses 1-7, wherein the first pattern of traces is operatively configured as a sensor.

Clause 9: The fluid-fillable circuit assembly according to any of clauses 1-8, wherein the sensor is configured to generate an electrical parameter that can be correlated to a structural parameter of the circuit.

Clause 10: The fluid-fillable circuit assembly according to any of clauses 1-9, wherein the electrical parameter includes at least one of an inductance, a current, a resistance, a voltage, a capacitance, an electromagnetic field, and an electromagnetic flux, or combinations thereof.

Clause 11: The fluid-fillable circuit assembly according to any of clauses 1-10, wherein the structural parameter includes at least one of a strain, a stress, a pressure, and a dimension, or combinations thereof.

Clause 12: The fluid-fillable circuit assembly according to any of clauses 1-11, further including a conduit configured to selectively enable fluid communication with the first fluid-fillable cavity, such that the first fluid-fillable cavity can be selectively inflated and deflated with a fluid through the conduit.

Clause 13: The partially fluid-fillable circuit assembly to any of clauses 1-12, further including a valve, wherein the valve is configured to selectively disenable fluid communication throughout the partially fluid-fillable circuit assembly.

Clause 14: The partially fluid-fillable circuit assembly to any of clauses 1-13, wherein the first fluid-fillable cavity defines a volume that changes as the first fluid-fillable cavity is selectively inflated and deflated.

Clause 15: The partially fluid-fillable circuit assembly to any of clauses 1-14, wherein the fluid is compressible.

Clause 16: The partially fluid-fillable circuit assembly to any of clauses 1-15, wherein a third portion of the layup includes a second sealed perimeter that, along with the at least one surface defined by the layup, defines a second fluid-fillable cavity.

Clause 17: The partially fluid-fillable circuit assembly to any of clauses 1-16, wherein the conduit and the valve are further configured to selectively enable fluid communication between the first fluid-fillable cavity and the second fluid-fillable cavity, such that the second fluid-fillable cavity can be selectively inflated and deflated with the first fluid-fillable cavity.

Clause 18: The partially fluid-fillable circuit assembly to any of clauses 1-17, wherein the partially fluid-fillable circuit assembly is configured to be at least partially contained within a housing.

Clause 19: The partially fluid-fillable circuit assembly to any of clauses 1-18, wherein the housing is a wearable article.

Clause 20: The partially fluid-fillable circuit assembly to any of clauses 1-19, wherein the wearable article is a shoe.

Clause 21: The partially fluid-fillable circuit assembly to any of clauses 1-20, wherein the wearable article is a joint monitoring sleeve.

Clause 22: The partially fluid-fillable circuit assembly to any of clauses 1-21, wherein the joint is at least one of a knee, an elbow, an ankle, a wrist, a knuckle, a shoulder, a vertebrae, and a hip.

Clause 23: The partially fluid-fillable circuit assembly to any of clauses 1-22, wherein an electrode is coupled to the first fluid-fillable cavity, and wherein inflating the first fluid-fillable cavity selectively biases the electrode against a user wearing the joint monitoring sleeve.

Clause 24: The partially fluid-fillable circuit assembly to any of clauses 1-23, wherein the electrode is configured to compress the cavity when a potential is applied to the electrode.

Clause 25: The partially fluid-fillable circuit assembly to any of clauses 1-24, wherein the biasing is caused by an electromagnetic attraction and/or an electromagnetic repulsion of the electrode relative to the conductive layer.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g.,“a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

What is claimed is:

1. A partially fluid-fillable circuit assembly, comprising:
a layup comprising:
a substrate layer;
a deformable conductor; and
an encapsulation layer covering the deformable conductor;
wherein a first portion of the layup comprises a sealed perimeter that, along with at least one surface defined by the layup, defines a first fluid-fillable cavity, wherein a second portion of the layup is unitized, and wherein the cavity defined by the first portion of the layup functions as a reservoir.

2. The partially fluid-fillable circuit assembly of claim 1, wherein the deformable conductor defines a first pattern of traces through the first portion of the layup.

3. The partially fluid-fillable circuit assembly of claim 2, wherein the deformable conductor defines a second pattern of traces through the second portion of the layup, and wherein an electronic component is coupled to the layup and electrically coupled to the first pattern of traces and the second pattern of traces.

4. The partially fluid-fillable circuit assembly of claim 2, wherein the first pattern of traces is operatively configured as a sensor that generates an electrical parameter correlated to a structural parameter of the circuit.

5. The partially fluid-fillable circuit assembly of claim 4, wherein the electrical parameter comprises at least one of an inductance, a current, a resistance, a voltage, a capacitance, an electromagnetic field, and an electromagnetic flux, or combinations thereof.

6. The partially fluid-fillable circuit assembly of claim 4, wherein the structural parameter comprises at least one of a strain, a stress, a pressure, and a dimension, or combinations thereof.

7. The partially fluid-fillable circuit assembly of claim 1, further comprising a conduit configured to selectively enable fluid communication with the first fluid-fillable cavity and a valve configured to selectively disenable fluid communication, such that the first fluid-fillable cavity can be selectively inflated and deflated with a fluid through the conduit.

8. The partially fluid-fillable circuit assembly of claim 7, wherein the conduit is formed in the second portion of the layup and the first fluid-fillable cavity defines a volume that changes as the first fluid-fillable cavity is selectively inflated and deflated with a compressible fluid to adjust pressure within the cavity.

9. The partially fluid-fillable circuit assembly of claim 7, wherein a third portion of the layup comprises a second sealed perimeter that defines a second fluid-fillable cavity, and wherein the conduit is further configured to selectively enable fluid communication between the first fluid-fillable cavity and the second fluid-fillable cavity.

10. The partially fluid-fillable circuit assembly of claim 7, wherein the valve is configured to selectively enable fluid communication between multiple fluid-fillable cavities.

11. The partially fluid-fillable circuit assembly of claim 1, wherein the partially fluid-fillable circuit assembly is configured to be contained within a housing.

12. The partially fluid-fillable circuit assembly of claim 11, wherein the housing is a wearable article.

13. The partially fluid-fillable circuit assembly of claim 12, wherein the wearable article is a shoe.

14. The partially fluid-fillable circuit assembly of claim 12, wherein the wearable article is a joint monitoring sleeve configured to monitor at least one of a knee, an elbow, an ankle, a wrist, a knuckle, a shoulder, a vertebrae, and a hip.

15. The partially fluid-fillable circuit assembly of claim 14, wherein an electrode is coupled to the first fluid-fillable cavity, and wherein selectively inflating the first fluid-fillable cavity selectively biases the electrode against a user wearing the joint monitoring sleeve.

16. The partially fluid-fillable circuit assembly of claim 15, wherein the biasing is caused by an electromagnetic attraction and/or an electromagnetic repulsion of the electrode relative to a conductive layer.

17. The partially fluid-fillable circuit assembly of claim 14, wherein the joint monitoring sleeve comprises the second fluid-fillable cavity positioned to monitor joint movement when the sleeve is worn by a user.

18. The partially fluid-fillable circuit assembly of claim 1, wherein the deformable conductor defines a first pattern of traces through the first portion of the layup and a second pattern of traces through the second portion of the layup, wherein an electrode is coupled to the layup and electrically coupled to the first pattern of traces and the second pattern of traces.

19. The partially fluid-fillable circuit assembly of claim 18, wherein the electrode is positioned on a first portion of the first fluid fillable cavity, wherein the partially fluid-fillable circuit assembly further comprises a conductive layer positioned on a second portion of the first fluid fillable cavity, and wherein the first fluid fillable cavity is filled with a dielectric fluid to enable electromagnetic interaction between the electrode and the conductive layer.

20. The partially fluid-fillable circuit assembly of claim 19, wherein the electrode is configured to compress the cavity when a potential is applied to the electrode.
